(19) United States
(12) Patent Application Publication
(10) Pub. No.: US 2026/0248773 A1
(43) Pub. Date: Aug. 27, 2026

(54) POTASSIUM CHANNEL BLOCKERS OR DERIVATIVES THEREOF FOR PREVENTING, ALLEVIATING, AND/OR TREATING TISSUE DYSFUNCTION CAUSED BY TOXIC INSULTS

(71) Applicant: University of Rochester, Rochester, NY (US)

(72) Inventors: Mark Noble, Rochester, NY (US); Jonathan Leckenby, Rochester, NY (US)

(21) Appl. No.: 18/992,254

(22) PCT Filed: Jul. 7, 2023
(86) PCT No.: PCT/US2023/069773

§ 371 Date: Jan. 8, 2025

Related U.S. Application Data
(60) Provisional application No. 63/367,934, filed on Jul. 8, 2022.
(60) Provisional application No. 63/380,143, filed on Oct. 19, 2022.
(60) Provisional application No. 63/490,267, filed on Mar. 15, 2023.

(51) Int. Cl. A61K 31/4409; A61P 39/02

(52) U.S. Cl. CPC A61K 31/4409; A61P 39/02

(57) ABSTRACT

The present invention provides compositions and methods of preventing, alleviating, or treating a subject with a toxic agent-induced injury. The present invention also provides compositions and methods of preventing, alleviating, or treating a tissue damage caused by exposure to a toxic agent or restoring at least a portion of normal tissue function. In some embodiments, the methods include administering to the subject a therapeutically effective amount of a pharmaceutical composition comprising at least one potassium channel blocker. In some embodiments, the potassium channel blocker comprises 4-aminopyridine, a derivative thereof, or a combination thereof. In some embodiments, the pharmaceutical composition can be administered with an additional therapeutic agent, such as an anticonvulsant.

### CROSS-REFERENCE TO RELATED APPLICATIONS

This application claims priority to U.S. Provisional Patent Application No. 63/367,934, filed on Jul. 8, 2022, U.S. Provisional Patent Application No. 63/380,143, filed on Oct. 19, 2022, and U.S. Provisional Patent Application No. 63/490,267, filed on Mar. 15, 2023, the contents of each of which are incorporated by reference herein in their entirety.

### BACKGROUND OF THE INVENTION

Pathological changes caused by exposure to toxic insults can create devastating clinical conditions for which there are currently no effective treatments. The problems caused by toxic insults thus represent major unmet medical needs. Damage can occur to many different tissues, can have effects in which onset is rapid, delayed, or a combination of the two, and in which recovery may or may not eventually occur if the condition is left untreated.

Particularly problematic are toxic insults that do not occur in nature and that require some type of industrial process in order to be generated. Such toxic insults are of several different types (e.g., industrial chemicals, chemotherapeutic agents, toxic agents of warfare). The most frequent exposures involve non-biological chemical substances that did not exist prior to the development of the chemical industry. Although the history of the chemical industry dates back thousands of years, large-scale production of chemicals first developed in the 19th century. The chemical industry began to emerge in association with the industrial revolution beginning in the late 18th century and began to accelerate in the 19th century when the production of synthetic dyes from coal tar became possible. The chemical industry then started its explosive growth in the first half of the 20th century and has undergone accelerated growth since then.

The development of the chemical industry is one of the factors that led to exposure of people to increasing numbers of chemicals that never existed in evolutionary history, which is conceptually important in understanding the novelty of the present invention. The fact that these chemicals were not part of evolutionary history means that there was no evolutionary selection for biological protective strategies. This is a very different situation than exists for trauma-based injuries, such as blunt force or sharp force injuries, or biological trauma, such as myocardial infarctions or other examples of ischemic injuries, or even exposure to toxic chemicals that occur through natural biological processes or that already exist in nature.

Humans have made multiple tens of millions of different chemicals, with the vast majority generated over the last few decades. It is estimated that another novel substance is either isolated or synthesized every few seconds, with the most recent ten million new compounds synthesized in a single year. Man-made chemicals are used for many purposes and not all are toxic, but an important subset of these chemicals is produced specifically to take advantage of their toxic properties.

For example, chemicals such as pesticides and avicides and herbicides are specifically developed in order to have toxic effects on a variety of organisms. It is thus not surprising that such chemicals can also be harmful to humans. Other chemicals, such as chemicals involved in industrial processes or developed for the purpose of enabling chemical reactions, or generating different kinds of materials, are accidentally toxic. Other toxic insults, such as irradiation, may be developed for the purposes of sterilization by killing of pathogens, for cancer treatment, or for military applications, and have the capacity to be intentionally toxic. Exposure to radiation also can be accidentally toxic, for example in the case of accidental exposures.

Perhaps the most conspicuous of the chemicals that cause tissue damage are those that are used to kill cancer cells, and that are intentionally applied to millions of people every year. These chemicals are produced specifically for the purpose of killing mammalian cells. Although the desired goal is to selectively target cancer cells, such selectivity rarely exists, and many normal cells can be harmed by the majority of chemotherapeutic agents. This is even the case for newer drugs that have been developed to try and increase immune attack against cancer cells, but which have been found to also exhibit unexpected toxicities. Other examples of such chemicals are agents developed for agricultural purposes, such as pesticides and insecticides, or agents developed for use in military purposes (such as nerve gases and other incapacitating agents).

In regards to the specific category of agents used to treat cancer, one example (among many) of a clinically important side effect of exposure to toxic chemicals is chemotherapy-induced peripheral neuropathy (CIPN). It is estimated that 30-70% of the 650,000 patients treated with chemotherapy every year in the United States develop symptoms of CIPN that range from numbness and tingling in the hands and feet, to burning pain, muscle weakness and loss of coordination. CIPN is a leading cause for non-compliance, reduced quality of life, and lower cancer survival rates. Patients who develop symptomatic CIPN also have an associated $20,000 increase in overall treatment cost in comparison to patients that do not develop symptoms and are also more likely to suffer relapse of their cancer.

One of the cancers in which CIPN is well-studied is breast cancer, which was the most common cancer in women worldwide, in 2018, accounting for more than a quarter of all cancers. Of the sub-types of breast cancer, hormone resistant tumors have the poorest prognosis. Surgery to remove tumors and affected lymph nodes is effective in early disease, but the mainstay of treatment in resistant tumors is with neoadjuvant chemotherapy. One of the preferred chemotherapy agents used in this patient group are taxanes, of which paclitaxel (PTX, most commonly used in the form of Taxol®) is most common. PTX is an effective agent at treating hormone resistant metastatic breast cancer. The problem is that PTX frequently induces CIPN and other toxic side effects on normal tissues (such as nephrotoxicity and damage to the central nervous system). Currently, there are no established treatments available for CIPN patients either to prevent the development of CIPN without blocking the effects of the chemotherapeutic agents themselves, or for successfully treating CIPN after it has developed (where treatment may represent a treatment that reverses symptoms caused by the damage, prevention or repair of the damage, or a combination of any of these characteristics). Pre-clinical studies are investigating multiple compounds to prevent or treat CIPN by blocking ion channels, targeting inflammatory cytokines, and combating oxidative stress, yet the results have not been encouraging.

There are also multiple other tissues besides peripheral nerve that are damaged by therapies used in the treatment of cancer. For example, chemotherapeutic agents and irradiation can damage the heart, lungs, the central nervous system, the gut, hair follicles, skin, the immune system, cells in the mouth, cells in the reproductive system common blood forming cells in the bone marrow and multiple other tissues.

It is an unmet medical need that there are no effective treatments for any tissue damage caused by exposure to toxic insults, such as chemotherapy, radiation, and/or toxic chemicals. These toxic insults can cause many different types of damage, and existing therapies are usually limited in their application. For example, chelation therapy for exposure to toxic metals (e.g., methylmercury, lead, etc.) has no utility in the treatment of chemotherapy-induced damage.

The medical needs represented by the tissue damage caused by cancer treatment represents a critical unmet medical need. Damage is caused in many instances where patients are faced with the difficult choice of accepting the possibility of undergoing toxic reactions to treatment or allowing the cancer to grow unchecked. Frequently what happens is that patients are treated with increasing dosages of chemotherapeutic agents, irradiation, or both until the toxicity reaches levels that can no longer be endured. These are called dose-limiting toxicities because they represent a point at which the aggressiveness of the anti-cancer therapy can no longer be increased because the doses of chemotherapy or radiation required to kill cancer cells cause unacceptable levels of damage to normal tissue and unacceptable physical symptoms. This can require decreasing the dose of the cancer treatment, or stopping it entirely, thus limiting its efficacy. Being able to prevent the toxicities caused by the chemicals used to treat cancer provides major benefits in cancer care, but current abilities to offer such benefits are inadequate to the medical need.

The medical needs represented by the tissue damage caused by exposure to toxic chemicals in the environment represent another critical unmet medical need. Damage can be caused in instances where individuals are exposed, for example, to individual chemicals used for a wide variety of industrial purposes, such as manufacturing or agricultural purposes. Examples of other exposures may include contamination of water supplies, for example due to chemical spills or fracking, or other means of exposure in the environment. For example, the generation of toxic insults in the vicinity of burn pits can cause the manifestation of damage to a variety of tissues. Other examples may include side effects of exposure to chemicals used for therapeutic purposes other than cancer treatment, such as the side effects of exposure to fluoroquinolone antibiotics. In these, and many other similar types of exposures, individuals may exhibit multiple types of symptoms due to exposure to a variety of different types of toxic insults of human-generated origin. Being able to prevent, reverse, ameliorate, or otherwise treat the toxicities caused by such toxic insults would provide major benefits in medical care, but current abilities to offer such benefits are inadequate to the medical need.

There are many challenges that interfere with the development of treatments for the effects of exposure to toxic insults. One of the particularly challenging questions is that of when treatment needs to be started. Does it need to be started within a short period after the exposure? If so, this may be suitable in situations where the time of exposure is known but it does not offer the opportunity to treat damage that already exists. In practice, the goal of treating existing damage is a very important one. Another challenge that is important in the treatment of established damage is whether the treatment is going to provide symptomatic relief that is dependent upon the constant availability of the drug or whether it actually can provide and promote durable recovery that enables a maintenance of benefit after the treatment is discontinued. All of these goals, of being able to start treatment after damage and/or symptoms are apparent, of providing symptomatic relief and of promoting durable recovery, are important ones. Developing treatments that offer the benefits of durable recovery and that can be initiated after the side effects are already present, however, is where some of the greatest challenges exist in developing treatments for the effects of exposure to toxic agents.

Another type of toxic insult is exposure to high doses of radiation at various locations along the electromagnetic spectrum at intensities sufficient to cause tissue damage. Examples of such insults include heat-generating wavelengths in the infrared range, radiation in the ultraviolet range, and most importantly, radiation toxicity representing ionizing radiation, most commonly in the alpha, beta, gamma, delta, and theta wavelengths, which causes damage at the chromosomal and cellular levels.

The most frequent source of radiation toxicity is treatment for cancer, as irradiation is used in the treatment of many different kinds of cancers. The radiation can be targeted with precision to specific regions of the body, and also can be used irradiate larger areas, right up to the use of whole body irradiation. Radiation toxicity also can occur in other situations, such as accidents at nuclear power plants and in military situations, such as the use of weapons that emit toxic levels of radiation.

As discussed, damage caused by non-biological chemicals or by radiation toxicity can occur in many different tissues and can manifest in many ways. For example, the normal cells that can be injured by chemotherapeutic agents include, but are not limited to, blood-forming cells in the bone marrow, hair follicles, cells in the mouth, digestive tract, reproductive system, and cells in the heart, kidneys, bladder, salivary glands, auditory system, visual system, lungs, and nervous system. Side effects of treatment with chemotherapeutic agents include, but are not limited to, fatigue, hair loss, easy bruising and bleeding, infection, anemia (low red blood cell counts), mouth, tongue, and throat problems such as sores and pain with swallowing, peripheral neuropathy or other nerve problems, such as numbness, tingling, and pain, urine and bladder changes and kidney problems, weight changes, such as those caused by cachexia, skeletal muscle atrophy and/or fibrotic changes, damage to heart muscle, chemo brain (which can affect concentration and focus), vascular damage, fertility problems (with effects on germ cells), anaemia, diarrhea, nausea, vomiting, infections, neurological changes, (e.g., National Cancer Institute: Chemotherapy Side Effects Sheets, cancer.gov/cancertopics/coping/physicaleffects/chemo-side-effects.)

In addition to the complexity associated with damage to different tissues, the timing at which toxic side effects are seen also can occur over a wide range of time. The side effects of exposure to toxic insults are classified as acute (within days), early delayed (within weeks), and late delayed toxicities (within months to years). As examples of the problems caused by exposure to toxic insults, a subset of pathological outcomes is presented. The choice of these illustrative outcomes is understood to offer non-limiting examples of this general class of problem, with examples chosen due to being particularly well studied.

One example of damage caused by toxic insults is a collection of conditions that are included in the broad grouping of peripheral neuropathies, which include CIPN as a subset. The broad class of changes classified as peripheral neuropathies is identified by symptoms presented, such as changes in sensation in peripheral nerves, but the underlying causes and mechanisms vary over a broad range. For example, even though expression of some of the shared symptoms of peripheral neuropathies occurs in many different situations, there is little or no reason to believe that the underlying causes and pathologies are the same even for neuropathies caused by biological afflictions, such as diabetic neuropathy, neuropathic pain associated with spinal stenosis, peripheral neuropathy in autoimmune diseases, such as Guillain Barre Syndrome, neuropathic pain following spinal cord injury or stroke. The underlying causes and pathologies for neuropathies caused by toxic insults represent still a different broad category of afflictions that are united by the sharing of a symptom rather than by being caused by shared mechanisms or being treatable by shared approaches.

The deficiency in using the designation of peripheral neuropathy as an indicator of a specific pathological process is even more so the case for neuropathic syndromes caused by exposure to toxic insults. There are many industrially-produced chemicals that can cause exposed individuals to exhibit symptoms that are described under the umbrella term of peripheral neuropathy, but expression of such symptoms has little or no bearing in understanding causation, pathophysiology or treatment of the clinical problem. Thus, it is understood that the term peripheral neuropathy is used only to indicate that a person expresses symptoms that would lead to their inclusion in this broad and multi-membered category, but that this term is not associated with specific causes or types of damage. There are many types of insults that can lead to outcomes that are collectively referred to as neuropathies, although that does not mean that the neuropathies are the same in respect to their detailed nature, their pathogenesis or their treatment.

The example of neuropathies including a broad range of different disturbances, even when they are generated by exposure to toxic agents, also extends to damage to other tissue. Toxic insults can cause a variety of different kinds of damage to the central nervous system, to the immune system, to the gut, to the heart, to lungs and to multiple other tissues. For example, damage to the central nervous system caused by exposure to chemotherapeutic agents, radiation or industrial toxins can cause damage to myelin, damage to neurons, activation of astrocytes, promotion of inflammation and a variety of cognitive and neurological manifestations. The variety of damage that is done underscores the challenge of developing effective treatments for this class of problem, because there are so many different kinds of damage that can occur. Analysis of chemotherapeutic agents provides a particularly powerful example of this problem because of the wide variety of types of damage that they can cause and the ability to use these agents as tests of the hypothesis that a particular therapeutic strategy can be applicable to multiple types of damage in multiple tissues.

Thus, there is a need in the art for new compositions and methods for preventing, alleviating, and/or treating tissue dysfunction caused by toxic insults. The present invention satisfies the need in the art.

### SUMMARY OF THE INVENTION

In one aspect, the present invention provides a method of preventing, alleviating, or treating a tissue damage or tissue dysfunction caused by a toxic insult in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In one aspect, the present invention provides a method of reducing or reversing a tissue damage, oxidation damage, scarring, or any combination thereof caused by a toxic insult in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In one aspect, the present invention provides a method of restoring, improving, or enhancing at least a portion of tissue function that was reduced by a toxic insult in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In one aspect, the present invention provides a method of restoring, improving, or enhancing myelination that was reduced or modulated by a toxic insult in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In one aspect, the present invention provides a method of preventing, alleviating, or treating a mitochondrial damage or mitochondrial dysfunction caused by a toxic insult in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In one aspect, the present invention provides a method of preventing, alleviating, or treating an axonal damage or axonal dysfunction caused by a toxic insult in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In one aspect, the present invention provides a method of preventing, alleviating, or treating at least one gait abnormality caused by a toxic insult in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In one aspect, the present invention provides a method of enhancing tissue regeneration, cell survival, or a combination thereof in a subject in need thereof who was exposed to at least one toxic insult, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In various embodiments, the at least one potassium channel blocker comprising 4-aminopyridine, a derivative of 4-aminopyridine, or a combination thereof. In some embodiments, the derivative of 4-aminopyridine is a compound having the structure of Formula (I)

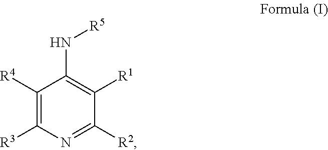

or an analog, a racemate, a tautomer, an isomer, an enantiomer, a diastereomer, a prodrug, or a pharmaceutically acceptable salt thereof. In some embodiments, R¹, R², R³, R⁴, and R⁵ are each independently selected from hydrogen, halogen, C₁-C₆ alkyl, amine, hydroxyl, alkoxy, carboxyl, or any combination thereof. In some embodiments, R¹, R², R³, R⁴, and R⁵ are optionally substituted.

In some embodiments, the tissue is a kidney tissue, liver tissue, heart tissue, lung tissue, brain tissue, central nervous system tissue, peripheral nerve tissue, gastrointestinal tract tissue, gut tissue, visual system tissue, auditory system tissue, skin tissue, bladder tissue, reproductive system tissue, hematopoietic system tissue, musculoskeletal tissue, or any combination thereof.

In some embodiments, the tissue function is a motor function, sensory function, cognitive function, visual function, auditory function, kidney function, hematopoietic system function, normal skin function, salivary gland function, liver function, gall bladder function, gastrointestinal (GI) function, sexual function, or any combination thereof.

In some embodiments, the tissue damage is a kidney tissue damage, liver tissue damage, heart tissue damage, lung tissue damage, brain tissue damage, central nervous system damage, peripheral nerve tissue damage, peripheral neuropathy, nephropathy, chemotherapy-induced peripheral neuropathy (CIPN), radiation-induced peripheral neuropathy (RIPN), chemotherapy-induced nephrotoxicity (CINT), chemotherapy-induced neutropenia, radiation-induced neutropenia, gastrointestinal tract tissue damage, gut tissue damage, visual system tissue damage, auditory system tissue damage, skin tissue damage, bladder tissue damage, reproductive system tissue damage, hematopoietic system tissue damage, or any combination thereof. In some embodiments, the chemotherapy-induced peripheral neuropathy (CIPN) is a CIPN caused by taxane agent (P-CIPN), CIPN caused by cisplatin treatment (CisIPN), CIPN caused by an anti-cancer agent, CIPN caused by a platinum-based antineoplastic agent, CIPN caused by a vinca alkaloid agent, CIPN caused by an epothilone agent, CIPN caused by a proteasome inhibitor, CIPN caused by an immunomodulatory drug, or any combination thereof.

Thus, in one aspect, the present invention provides a method of preventing, alleviating, or treating a chemotherapy-induced peripheral neuropathy (CIPN) in a subject in need thereof, the method comprising administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In some embodiments, the tissue damage is a multi-tissue damage, multi-organ tissue damage, or any combination thereof.

In some embodiments, the toxic insult is an acute toxic insult, chronic toxic insult, or any combination thereof.

In some embodiments, the treatment can be started at the time of the initiating insult, during continued exposure to toxic insult, or in the treatment of tissue damage that develops with a delay after the exposure to the toxic insult has ended.

In some embodiments, the toxic insult comprises an exposure to damaging levels of radiation along the electromagnetic spectrum.

In some embodiments, the toxic insult is a non-biological substance, non-naturally occurring compound, toxin, agent used in treating cancer, chemotherapy agent, biological response modifier, radiation, or any combination thereof. In some embodiments, the chemotherapy agent comprises a platinum-based antineoplastic agent, vinca alkaloid agent, epothilone agent, taxane agent, proteasome inhibitor, immunomodulatory drug, or any combination thereof. In some embodiments, the toxic insult comprises at least one environmental toxicant. In some embodiments, the toxic insult comprises at least one industrially-produced compound. In some embodiments, the radiation comprises a radiation from a radioactive cancer treatment, radiation from a nuclear energy accident, radiation from a nuclear waste exposure, radiation from a use of radioactive substances in military applications, or any combination thereof.

In some embodiments, the toxic insult comprises at least a first toxic insult and a second toxic insult. In some embodiments, the first toxic insult is a chemotherapy agent, radiation, or a combination thereof and the second toxic insult is a chemotherapy agent, radiation, or a combination thereof.

In some embodiments, the therapeutically effective amount of the pharmaceutical composition is administered to the subject before the toxic insult.

In some embodiments, the therapeutically effective amount of the pharmaceutical composition is administered to the subject at the time of the toxic insult.

In some embodiments, the therapeutically effective amount of the pharmaceutical composition is administered to the subject after the toxic insult.

In some embodiments, the therapeutically effective amount of the pharmaceutical composition is administered to the subject more than once. In some embodiments, the therapeutically effective amount of the pharmaceutical composition is repeatedly administered to the subject for between about 1 day to about 100 years.

In some embodiments, the therapeutically effective amount of the pharmaceutical composition is administered to the subject systematically, locally, or a combination thereof.

In some embodiments, the therapeutically effective amount of the pharmaceutical composition is administered to the subject by an intraperitoneal injection, intravenous injection, intramuscular injection, intrathecal injection, subcutaneous injection, sublingual administration, inhalation, oral administration, transdermal administration, administration to an outer portion of the body in the form of a liquid, administration to an outer portion of the body in the form of a salve, administration to an outer portion of the body in the form of a bandage, or any combination thereof.

In some embodiments, the therapeutically effective amount of the pharmaceutical composition is administered to the subject at a dose of between about 1 mg/day to about 1,000 mg/day of the potassium channel blocker. In some embodiments, the therapeutically effective amount of the pharmaceutical composition is administered to the subject at a dose of between about 2.5 mg/day to about 40 mg/day of the potassium channel blocker. In some embodiments, the therapeutically effective amount of the pharmaceutical composition is administered to the subject at a dose of between about 40 mg/day to about 100 mg/day of the potassium channel blocker.

In some embodiments, the therapeutically effective amount of the pharmaceutical composition is co-administered with at least one anticonvulsant agent or a composition thereof.

In some embodiments, the at least one anticonvulsant agent is barbiturate, benzodiazepine, bromide, carbamate, carboxamide, fatty acid, fructose or a derivative thereof, γ-aminobutyric acid (GABA) or an analog thereof, hydantoin, oxazolidinedione, proprionate, pyrimidinedione, pyrrolidine, succinimide, sulfonamide, triazine, urea, valproylamide, or any combination thereof.

In various embodiments, the method further enhances cell survival, reduces scarring, or any combinations thereof.

In various embodiments, the method further enhances a repair or regeneration of endogenous stem cells, enhances a repair or regeneration of transplanted stem cells, enhances a repair or regeneration of progenitor cells, promotes a neural cell generation, enhances cell survival, reduces scarring, decreases lesion size, decreases oxidative damage, or any combinations thereof.

In one aspect, the present invention also provides a method of identifying a subject responsive to 4-aminopyridine administration to prevent, alleviate, or treat a tissue damage or tissue dysfunction caused by a toxic insult in a subject in need thereof.

In various embodiments, the method comprises the steps of: a) administering to the subject between 1 to 5 therapeutically effective amounts of a pharmaceutical composition comprising a 4-aminopyridine, derivative of 4-aminopyridine, or a combination thereof; b) evaluating the symptoms of the tissue damage or tissue dysfunction caused by the toxic insult in the subject; and c) identifying the subject as responsive to 4-aminopyridine administration to prevent, alleviate, or treat the tissue damage or tissue dysfunction caused by a toxic insult when the symptoms of the tissue damage or tissue dysfunction caused by the toxic insult in the subject improved.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1H, depicts representative results demonstrating that 4-aminopyridine (4-AP) conferred protection against developing a chemotherapy-induced peripheral neuropathy (CIPN) and mitochondrial damage caused by clinically relevant doses of paclitaxel (PTX). 16 week old C7BL/6 female mice were given a single dose of 35 mg/kg PTX (i.p.). Animals were separated into control (saline, i.p., n=4) or treatment group (2 mg/kg 4-AP daily, i.p., n=4). Pre-treatment Catwalk scores were compared to 21 days post-PTX injection. FIG. 1A depicts representative nerve conduction studies that showed significant increases in latency in the control group over the study. In 4-AP treated mice, there was a significantly smaller increase in latency. FIG. 1B depicts representative nerve conduction studies that showed significant decreases in velocity in the control group over the study. In 4-AP treated mice, there was a significantly smaller decrease in velocity. FIG. 1C depicts representative catwalk analysis that demonstrated significant increases in swing phase in control mice, but no significant changes in the 4-AP treated group (p<0.05; p<0.01; p<0.001). FIG. 1D depicts representative Catwalk analysis that demonstrated significant increases in stance phase in control mice (indicative of gait abnormalities), but no significant changes in the 4-AP treated group (*p<0.05; **p<0.01; ***p<0.001). FIG. 1E depicts representative results demonstrating that 4-AP treatment also decreases PTX-induced damage to mitochondria. Mitochondria were scored as healthy or sick, and data were analyzed with a contingency Chi test of overall 4-AP vs NaCl. Mitochondria were scored from 64 (saline) or 57 (4-AP) randomly selected axons, with 172 (saline) or 177 (4-AP) total mitochondria scored. FIG. 1F depicts a representative image of a healthy mitochondria. FIG. 1G depicts a representative image of a sick (i.e., not healthy) mitochondria. FIG. 1H depicts representative results demonstrating that mitochondria analyzed in saline-treated animals also showed a greater proportion of enlarged mitochondria, which generally were swollen and unhealthy in appearance (*p<0.05).

FIG. 2, comprising FIG. 2A through FIG. 2E, depicts representative results demonstrating that 4-AP prevented chemotherapy induced axonal damage. FIG. 2A depicts representative G ratio (axon:myelin area) data demonstrating that axons within the 4-AP treatment were better myelinated than the control group. FIG. 2B depicts representative circularity data demonstrating that axons within the 4-AP treatment also were more regularly structured than the control group. FIG. 2C depicts representative baseline appearance of sciatic nerve. FIG. 2D depicts representative appearance of control axons 3-weeks following one treatment of 35 mg/kg PTX. FIG. 2E depicts representative appearance of 4-AP (2 mg/kg daily) treated axons demonstrating thicker myelin and more regular structure. In this figure, “Control”=PTX+Saline.

FIG. 3, comprising FIG. 3A through FIG. 3I, depicts representative results demonstrating that 4-AP also conferred protection against developing CIPN when animals were repetitively exposed to PTX. 16-week-old C7BL/6 female mice were given four cycles of PTX treatment at doses of 35 mg/kg PTX (i.p.) at three-week intervals. It is demonstrated that 4-AP treatment prevents CIPN-related changes in peripheral nerve function caused by repetitive exposure to Paclitaxel (PTX). Mice were treated with PTX (35 mg/kg), every 3 weeks for 4 cycles. Mice also were treated either with saline or with 4-AP (0.5 mg/kg/day), weeks 1-12. PTX induces signs of CIPN. 4AP treatment significantly reduces mechanical allodynia (FIG. 3A), and reduces total number of jumps (FIG. 3C) (*p<0.05, **p<0.01, ***p<0.001, ****p<0.0001). 4-AP treatment significantly prevents the deterioration of gait and nerve conduction (FIG. 3D-FIG. 3I). (*p<0.05, **p<0.01, ***p<0.001, ****p<0.0001). Animals were separated into control (saline, i.p.) or treatment group treated with a dose of 4-AP 25% of that used in FIG. 1 and FIG. 2 (i.e., 0.5 mg/kg 4-AP daily, i.p.). Pre-treatment outcomes were compared to weeks 3, 6, 9, and 12 post-PTX injection. FIG. 3A depicts the ability of 4-AP treatment to prevent PTX-induced mechanical hypersensitivity as tested by application of von Frey filament testing, where the sensitivity to smaller filaments reveals increased sensitivity to painful stimuli. Outcomes were highly significant at Weeks 3, 6, 9 and 12. FIG. 3B and FIG. 3C show that 4-AP treatment also prevents development of the opposite symptoms of hyposensitivity to stimuli, using analysis of pawlifts (FIG. 3B) and jumping behavior (FIG. 3C) in response to cold-plate stimulus. Pawlift outcomes trended significant at Weeks 6 and 9 and were highly significant at Week 12. Jumping behavior was highly significant at Weeks 6, 9 and 12. FIG. 3D-FIG. 3F depict the ability of 4-AP to prevent PTX-induced gait abnormalities detected by Catwalk analyses, with significant effects at multiple time points. FIG. 3G-FIG. 3I depict the ability of 4-AP to prevent PTX-induced abnormalities in nerve conduction in the multiple outcomes of latency (FIG. 3G), amplitude (FIG. 3H) and velocity (FIG. 3I).

FIG. 4, comprising FIG. 4A through FIG. 4G, demonstrate the ultrastructural damage to the peripheral nervous system caused by PTX. The figures depict representative results demonstrating that 4-AP prevented chemotherapy-induced axonal damage even when chemotherapy was applied multiple times (35 mg/kg, every 3 weeks, 4 cycles of treatment) and the dose of 4-AP was only 25% of the dose used in FIG. 1 and FIG. 2, and was applied at 0.5 mg/kg instead of 2 mg/kg). In this figure, “Control”=PTX+Saline. It is demonstrated that 4-AP treatment prevents CIPN-related changes in peripheral nerve ultrastructure caused by repetitive exposure to Paclitaxel (PTX). Mice were treated with PTX (35 mg/kg), every 3 weeks for 4 cycles. Mice also were treated either with saline or with 4-AP (0.5 mg/kg/day), weeks 1-12. PTX induces a variety to deleterious changes in nerve ultrastructure, and 4-AP treatment significantly decreases these changes. (FIG. 4A) Overview of parameters studies. (FIG. 4B) Examples of structural changes. (FIG. 4C-FIG. 4E) Quantification of PTX and 4-AP effects on nerve ultrastructure. (FIG. 4F) Examples of mitochondrial ultrastructure. (FIG. 4G) Quantification of PTX and 4-AP effects on mitochondria. (*p<0.05, **p<0.01, ***p<0.001, ****p<0.0001). FIG. 4A summarizes the ultrastructural analyses of myelination conducted. Typically, CIPN results in axons that are less myelinated and are less circular. FIG. 4B depicts representative images of the changes in myelin-related parameters caused by PTX exposure and the benefits of 4-AP treatment. By directly comparing the electron micrographs after the animals completed 4 cycles of PTX treatment (described above), it is clear that the animals treated with 4-AP (0.5 mg/kg daily) have more circular and better myelinated axons than the control group, and are similar in appearance to naïve animals that have not received any PTX treatment (baseline). FIG. 4C depicts representative G ratio (axon:myelin area) data demonstrating that axons within the 4-AP treatment maintained better myelination than the control group (normal G-ratio value is 0.65). FIG. 4D depicts representative circularity data demonstrating that axons within the 4-AP treatment also were more regularly structured than the control group. FIG. 4E depicts representative data demonstrating the 4-AP decreases the frequency of degenerating myelin profiles. FIG. 4G depicts representative images of damaged mitochondria. FIG. 4H depicts that treatment with 4-AP prevents mitochondrial degeneration as determined by loss of mitochondrial membrane integrity and presence of vacuoles.

FIG. 5, comprising FIG. 5A through FIG. 5I, depicts representative results demonstrating that 4-AP also reverses the effects of repetitive PTX treatment on peripheral nerve, with 4-AP treatment commencing at 6 weeks when mice received their third exposure to PTX. It is demonstrated that 4-AP treatment reverses CIPN-related changes in peripheral nerve function and ultrastructure caused by repetitive exposure to Paclitaxel (PTX). Mice were treated with PTX (35 mg/kg), every 3 weeks for 4 cycles. Mice also were treated either with saline or with 4-AP (0.5 mg/kg/day), weeks during 6-12. PTX induces signs of CIPN. 4AP treatment significantly reduces mechanical allodynia (FIG. 5A), and reduces total number of jumps (FIG. 5B). 4-AP treatment significantly prevents the deterioration of gait and nerve conduction (FIG. 5C-FIG. 5H). (*p<0.05, **p<0.01, ***p<0.001, ****p<0.0001). 16-week-old C7BL/6 female mice were given four cycles of PTX treatment at doses of 35 mg/kg PTX (i.p.). Animals were separated into control (saline, i.p.) or treatment group treated with a dose of 4-AP (0.5 mg/kg 4-AP daily, i.p.), beginning at week 6 after signs of CIPN had developed. As above, this dose was 25% of that used in FIGS. 1 and 2. FIG. 5A depicts the effects of 4-AP treatment on PTX-induced mechanical hypersensitivity as tested by application of von Frey filament testing, where the sensitivity to smaller filaments reveals increased sensitivity to painful stimuli. Benefits began to be observed at Week 12. FIG. 5B shows that 4-AP treatment also reverses the opposite symptoms of hyposensitivity to stimuli, using analysis of jumping behavior (FIG. 5B) in response to cold-plate stimulus. Changes in jumping behavior were significant at Weeks 9 and 12. FIG. 5C-FIG. 5E depict the ability of 4-AP to reverse PTX-induced gait abnormalities detected by Catwalk analyses, with significant effects at weeks 9 (the first analysis after 4-AP treatment started) and 12 for swing time and regularity index, and outcomes for stance time trending towards significance at these same time points. FIG. 5F-FIG. 5H depict the ability of 4-AP to reverse PTX-induced abnormalities in nerve conduction after they are established by multiple rounds of PTX exposure in the multiple outcomes of latency (FIG. 5F), amplitude (FIG. 5G) and velocity (FIG. 5H). FIG. 5I depicts the ability of 4-AP to reverse PTX-induced myelin degeneration.

FIG. 6, comprising FIG. 6A through FIG. 6G, depicts results indicating that the use of 4-AP treatment to reverse effects of repetitive rounds of PTX exposure on peripheral nerve function is retained even after treatment ends. Such durable changes are indicative of pro-reparative effects of 4-AP treatment that extend beyond providing symptomatic relief that only is present during the time of treatment. It is demonstrated that 4-AP treatment durably reverses CIPN-related changes in peripheral nerve function and ultrastructure caused by repetitive exposure to Paclitaxel (PTX). Mice were treated with PTX (35 mg/kg), every 3 weeks for 4 cycles. Mice also were treated either with saline or with 4-AP (0.5 mg/kg/day), weeks 6-12. Mice were also followed for an additional 6 weeks after the completion of 4-AP treatment. 4-AP treatment significantly reduces mechanical allodynia (FIG. 6A), and reduces total number of jumps (FIG. 6C). 4-AP treatment significantly prevents the deterioration of gait and nerve conduction (FIG. 6D-FIG. 6G). (*p<0.05, **p<0.01, ***p<0.001, ****p<0.0001) In these experiments, 16-week-old C7BL/6 female mice were given four cycles of PTX treatment at doses of 35 mg/kg PTX (i.p.). Animals were then treated with the same dose and treatment regimen of 4-AP used in FIG. 5. Treatment with 4-AP ended at Week 12 after the first exposure to PTX and after 6 weeks of treatment with 4-AP, and animals were observed for an additional six weeks after this point (i.e., for 6 weeks with no 4-AP treatment). FIG. 6A depicts the effects of 4-AP treatment on PTX-induced mechanical hypersensitivity as tested by application of von Frey filament testing, where the sensitivity to smaller filaments reveals increased sensitivity to painful stimuli. Benefits began to be observed at Week 12 and were maintained at Weeks 15 and 18. FIG. 6B shows that 4-AP treatment also causes durable reversal the opposite symptoms of hyposensitivity to stimuli, using analysis of paw lifts (FIG. 6B) and jumping behavior (FIG. 6C) in response to cold-plate stimulus. FIG. 6D depicts the ability of 4-AP to durably reverse PTX-induced gait abnormalities as determined by the Catwalk Regularity Index, with benefits observed at Week 9 (i.e, after 3 weeks of 4-AP treatment) and retained for at least 6 weeks after 4-AP treatment ended. FIG. 6E depicts the ability of 4-AP to reverse PTX-induced abnormalities in nerve conduction latency after they are established by multiple rounds of PTX exposure. FIG. 6F-Figure G depict the ability of 4-AP to cause durable pro-reparative changes when used to treat established PTX-induced CIPN and tissue damage. The changes in G-ratio (FIG. 6F) and circularity (FIG. 6G) caused by repetitive PTX exposure are restored to normal at the 12-week time point, and these benefits are maintained at the 18-week time point (i.e, 6 weeks after treatment has ended).

FIG. 7, comprising FIG. 7A through FIG. 7D, depicts representative results demonstrating 4-AP reverses CIPN induced by clinically relevant doses of cisplatin, a chemotherapeutic agent with very different mechanisms of action than PTX. It is demonstrated herein that 4-AP treatment reverses CIPN-related changes in peripheral nerve function caused by repetitive exposure to cisplatin (CIS). Mice were treated with CIS (2 mg/kg/week for 8 weeks). Mice also were treated either with saline or with 4-AP (1 mg/kg/day), weeks 9-15. CIS induces signs of CIPN. 4AP treatment reverses weight loss (FIG. 7A), mechanical allodynia (FIG. 7B), and changes in nerve conduction (FIG. 7C and FIG. 7D). 16-week-old C7BL/6 female mice were treated with CIS (2.5 mg/kg, once weekly, i.p.) for eight weeks. Animals commenced treatment with 4-AP (1 mg/kg) at week 9, continuing treatment for a total of 6 weeks (n=8). FIG. 7A depicts representative results demonstrating the effect of 4-AP on body weight. As shown, mice treated with 4-AP beginning 9 weeks after the initiation of CIS treatment regained normal body weight. FIG. 7B depicts representative results demonstrating the effect of 4-AP on Von Frey filament testing. Analysis of hyperalgesia by Von Frey filament analysis showed a dramatic increase in sensitivity in CIS treated mice, and a restoration of normal levels of sensitivity with 4-AP treatment. The restoration of normal sensitivity was maintained for at least two weeks after treatment ended, well beyond the 12-16 hour time point when virtually all 4-AP would be expected to be cleared from the body, thus demonstrating durable recovery of function and indicating pro-reparative effects of the treatment. FIG. 7C depicts representative results demonstrating the effect of 4-AP on nerve impulse amplitude that was also restored by 4-AP treatment with improvements again retained for at least two weeks after treatment with 4-AP ended. FIG. 7D depicts representative results demonstrating the effect of 4-AP on nerve impulse velocity that was also restored by 4-AP treatment with improvements again retained for at least two weeks after treatment with 4-AP ended thus demonstrating durable recovery of function and indicating pro-reparative effects of the treatment.

FIG. 8, comprising FIG. 8A through FIG. 8C, shows that 4-AP co-treatment also prevents multiple aspects of histological kidney damage caused by PTX exposure. 16-week-old female C57BL/6 mice were inoculated with a triple negative murine breast cancer cell line E0771. When tumors were palpable, the mice were injected with either paclitaxel and saline, 4-AP (1 mg/kg), both, or saline as a control. Paclitaxel (35 mg/kg) was injected in all mice on day one and 4-AP or saline were injected daily. Mice were sacrificed on day 14 and perfused with 4% paraformaldehyde and harvested. Kidneys were removed, sectioned and stained with hematoxylin & eosin (H&E). H&E staining of saline treated (FIG. 8A), PTX treated (FIG. 8B), and PTX+4-AP treated (FIG. 8C) kidneys (FIG. 8, from left to right) revealed normal proximal and distal tubules in both saline and PTX+4-AP treated kidneys. Loss of brush borders (dark grey arrows), nuclear dropout (grey arrow), and blebbing of tubular epithelial cells (black arrows) were observed in PTX treated kidneys but not in kidneys isolated from animals treated with PTX+4-AP.

FIG. 9 depicts the ability of 4-AP treatment to prevent PTX-induced increases in a reactive inflammatory response in the brain, as detected by increased expression of glial fibrillary acidic protein (GFAP) in the corpus callosum. GFAP is increased in astrocytes in association with injury and inflammation. These experiments were conducted on the same mice as in FIG. 8. 16-week-old female C57BL/6 mice were inoculated with a triple negative murine breast cancer cell line E0771. When tumors were palpable, the mice were injected with either paclitaxel and water, 4-AP, both, or saline as a control. Paclitaxel (35 mg/kg) was injected on day one and 4-AP and water were injected daily. The mice were sacrificed on day 14 and their brains were perfused and harvested. Brains were cryosectioned coronally. The mouse brain sections were stained with anti-GFAP antibodies, followed by a fluorescent secondary, for analysis via immunofluorescence. Images were taken via a confocal microscopy and images were analyzed with Image-J. The data is shown as the area of tissue expressing GFAP, and show that PTX treatment is associated with increased expression of GFAP. In mice co-treated with 4-AP the increased expression of GFAP was greatly reduced.

FIG. 10, comprising FIG. 10A and FIG. 10B, depict the results of experiments demonstrating that protection from chemotherapeutic agents is specific to normal (non-transformed) cells, and does not extend to cancer cells. FIG. 10A depicts representative results demonstrating that concurrent treatment with 4-AP did not protect E0771 murine breast cancer (BC) cells from the effects of 5 days exposure to PTX in vitro, an important outcome as a treatment for toxic side effects of cancer treatments that also protected tumor cells would be of little clinical value. FIG. 10B shows that similar treatment of A549 lung cancer cells with 4-AP did not protect these cancer cells against the toxic effects of cisplatin.

### DETAILED DESCRIPTION

In the following description, chemotherapy-induced peripheral neuropathy (CIPN) is described as a particularly well studied non-limiting example of tissue damage caused by exposure to a variety of different types of toxic insults. Even the aggregation of some of these insults as chemotherapeutic agents is itself an oversimplification, however, because chemotherapeutic agents can have a wide range of targets and a wide range of molecular structures. Some of these agents, such as the taxanes and vinca alkaloids, are closely related to substances that exist in nature even though they are industrially produced for the purpose of treating cancer. In contrast, other agents, such as platinum-containing drugs (e.g., cisplatin and oxaliplatin) are much more akin to industrial chemicals that are generated for their toxic properties.

One of the best studied examples of a peripheral neuropathy-like syndrome induced by exposure to toxic chemicals is that induced by treatment with-neoplastic agents, referred to as chemotherapy-induced peripheral neuropathy (CIPN) (Hershman et al., 2014, J. Clin. Oncol., 32:1941-1967). CIPN is so frequent as to occur in 19-85% of treated individuals, with different frequencies dependent upon the components of the treatment (Fallon et al., 2013, Br. J. Anaesth., 111:105-111). Among the factors modulating the prevalence of CIPN is specific treatment agent used, with reported rates varying from 19% to more than 85% and is the highest in the case of platinum-based drugs (70-100%), taxanes (11-87%), thalidomide and its analogues (20-60%), and ixabepilone (60-65%) (Banach et al., 2016, Brain Behav., 7: e00558).

Chemotherapeutic agents can cause a variety of neuropathies, including, for example, large and small fiber, sensory, and/or motor, demyelinating and axonal, cranial and autonomic (e.g., Cioroiu et al., 2017, Curr. Neurol. Neurosci. Rep., 17:47). At a simplistic level, CIPN is often characterized as a predominantly sensory neuropathy that may be accompanied by motor and autonomic changes (Seretny et al., 2014, Pain, 155:2461-2470). In addition, painful sensations, including spontaneous burning, shooting or electric shock-like pain as well as mechanical or thermal allodynia or hyperalgesia frequently occur (Bernhardson et al., 2007, J. Pain Symptom Manag., 34:403-412). In severe cases, CIPN can lead to paresis, complete patient immobilization and severe disability (Mols et al., 2016, Eur. J. Cancer, 69:28-38) In general, sensory disorders occur more frequently than autonomic symptoms, which usually involve orthostatic hypotension, constipation, and altered sexual or urinary function (Mols et al., 2016, Eur. J. Cancer, 69:28-38).

Some compounds, such as paclitaxel and oxaliplatin, can cause acute neuropathy during or immediately after infusion (Argyriou et al. Clinical pattern and associations of oxaliplatin acute neurotoxicity: A prospective study in 170 patients with colorectal cancer (Cancer. 2013; 119:438-444)). In many other instances, in contrast, CIPN symptoms can emerge weeks or months after the completion of chemotherapy. Some patients experience paradoxical worsening and/or intensification of symptoms after the cessation of treatment (Starobova H et al., 2017, Front. Mol. Neurosci., 10:174). Affected individuals also may experience a worsening of mild neuropathy or development of new CIPN. Symptom severity is generally proportional to the cumulative dose of the drug (Maestri et al., 2005, Tumori, 91:135-138), but there are also other factors that can influence symptom severity, such as age, genetic factors, exposure to other chemicals or to radiation, and a range of other contributors.

Recent studies put the prevalence of CIPN at approximately 68.1% when measured in the first month after chemotherapy, 60.0% at 3 months, and as high as 30.0% after 6 months (Seretny et al., Pain. 2014; 155:2461-2470). Especially in the case of platinum-based anticancer agents and taxanes, CIPN may last several years after the completion of chemotherapy (Kerckhove et al., Front. Pharmacol. 2017; 8:86). The damage that occurs can vary between different chemotherapeutic compounds, and each of the compounds that can cause CIPN has been studied in some detail. Non-limiting illustrations of the complexity and severity of this problem are provided in the following examples.

The effects of chemotherapy on the nervous system vary among the different classes of drugs, depending on the specific physical and chemical properties of the drug used and its single or cumulative doses (Banach et al., Brain Behav. 2016, 7: e00558). Toxicity can occur even after a high single dose of treatment, or after cumulative exposure. Observed symptoms of damage to the peripheral nervous system vary in intensity and duration. They range from acute, transient thermal sensations to permanent changes in peripheral nerves accompanied by chronic pain and irreversible nerve damage (Seretny et al., Pain. 2014, 155:2461-2470). Six main categories of chemotherapeutic agents cause damage to the peripheral sensory, motor, and autonomic neurons, leading to CIPN development. These are the platinum-based antineoplastics (particularly oxaliplatin and cisplatin), the vinca alkaloids (particularly vincristine and vinblastine), the epothilones (ixabepilone), the taxanes (paclitaxel, docetaxel), the proteasome inhibitors (bortezomib) and immunomodulatory drugs (thalidomide) (Starobova H et al., Front. Mol. Neurosci. 2017, 10:17). Of these agents, the most neurotoxic classes are platinum-based drugs, taxanes, ixabepilone, thalidomide, and its analogues. The vinca alkaloids show less neurotoxity, but can cause multiple other toxicities.

One of the challenges in developing means of preventing damage caused by chemotherapeutic agents, and by toxic chemicals in general, is that multiple types of disruptions occur at the cellular level. Examples of multiple of these potential disruptions are provided in the illustrative examples that follow. At the general level, disruptions in normal cellular function that have been suggested to be relevant to the pathogenesis of peripheral nervous system damage include microtubule disruption, oxidative stress, mitochondrial damage, altered ion channel activity, myelin sheath damage, DNA damage, immunological processes and neuroinflammation. (Zajączkowska et al., 2019, Int. J. Mol. Sci., 20:1451; ncbi.nlm.nih.gov/pmc/articles/PMC6471666/). All of these changes have been suggested to be potentially important in CIPN but there is no indication as to whether any changes are of paramount importance in respect to pathogenesis or treatment. Nor have any of these suggestions been useful in development of either prophylactic or post-injury treatments, as discussed elsewhere in this application. In brief, targeting these changes in a specific manner has not led to the development of effective therapeutic agents and indicates that the discovery of such therapeutic agents is neither straightforward nor predictable.

Taxane-induced CIPN offers an example of the complexity of these insults and their difference from those caused by traumatic injury. The taxanes cause CIPN in 11-87% of patients (Banach et al., Brain Behav. 2016; 7: e00558.) Taxanes have many different effects on cell function. Paclitaxel is an excellent example of the complexity of the insults caused by taxanes and other types of toxic insults, and their difference from those caused by physical trauma. Some of these are thought to be direct effects while others may be indirect effects.

Taxanes are best known for disrupting microtubule function, which is thought to be associated with the development of CIPN (Gornstein et al., Exp. Neurol. 2017, 288:153-166). Taxanes also cause damage to mitochondria. This can cause oxidative stress, and the production of reactive oxygen species damages many cellular components, which can lead to impaired axonal transport due to these reasons as well as due to taxane effects on microtubules. These changes can have multiple effects, such as impaired signal transmission, inflammation, and damage to myelin. Of these changes, however, the damage to myelin showed the least evidence of causal linkage to CIPN (Areti et al., Redox Biol. 2014; 2:289-295; Bulua et al., J. Exp. Med. 2011; 208:519-533; Griffiths et al., J. Pain. 2015; 16:981-994; Duggett et al., Pain. 2017; 158:1499-1508). Still other types of damage associated with paclitaxel treatment includes damage to mitochondria, including mitochondrial swelling, vacuolation, and loss of structure of mitochondria (Flatters et al., Pain. 2006; 122:245-257; Xiao et al., Pain. 2012; 153:704-709).

Another suspected contributor to paclitaxel-induced CIPN is dysregulation of Ca2+ homeostasis (Siau et al., Anesth. Analg. 2006; 102:1485-1490; Yilmaz et al., Cell Calcium. 2017; 62:16-28; Kidd et al., J. Biol. Chem. 2002; 277:6504-6510; Mironov et al., J. Biol. Chem. 2005; 280:715-721). Paclitaxel can cause release of Ca2+ from mitochondria, seemingly by opening of mitochondrial permeability transition pores and rapid mitochondria depolarization, and also can cause release of Ca2+ from the endoplasmic reticulum. (Kidd et al., J. Biol. Chem. 2002; 277:6504-6510; Mironov et al., J. Biol. Chem. 2005; 280:715-721; Boehmerle et al., Proc. Natl. Acad. Sci. USA. 2006; 103:18356-18361; Li et al., Pain. 2017; 158:417-429). Another indicator of the potential contribution of increases in Ca2+ levels to CIPN is an increased expression of CaV3.2 channels in rats with paclitaxel treatment, and findings that suppression of these specific Ca2+ channels reverses hyperalgesia (Okubo et al., Neuroscience. 2011; 188:148-156). These results indicate that increases in Ca2+ are an important contributor to the development of paclitaxel-induced CIPN. As one of the properties of 4-AP is to activate high-voltage-activated Ca2+ channels and thus cause Ca2+ release, these studies would counter-indicate utility of 4-AP.

There is also altered expression and function of multiple other ion channels in paclitaxel-induced CIPN. The cation channels TRPV1 and TRPA1, which are important in pain signaling, are important in paclitaxel-induced CIPN (Hara et al., Pain. 2013; 154:882-889; Materazzi et al., Pflugers Arch. 2012; 463:561-569), and TRPA1 antagonists can relieve inflammation, cold allodynia and hyperalgesia induced by paclitaxel (Chen et al., Neuroscience. 2011; 193:440-451). Paclitaxel treatment also causes increases in the number of NaV1.7 channels, and blocking of this channel can attenuates hyperalgesia in rats. (Li et al., J. Neurosci. 2018; 38:1124-1136; Aromolaran et al., Mol Pain. 2017; 13:1744806917714693; Gheraldin et al., Neuroscience. 2010; 169:863-873). In addition, decreased expression of K+ channels causing the spontaneous activity of nociceptors was observed in the DRG in a paclitaxel-induced CIPN model (Zhang H et al., Anesthesiology. 2014; 120:1463-1475)

Another suggested contributor to the pathogenesis of paclitaxel-induced CIPN is inflammation. Paclitaxel exposure increases the production of pro-inflammatory cytokines (TNF alfa and IL-1 beta) and decreases anti-inflammatory cytokines (IL-4 and IL-10), leading to the attraction and activation of immune cells and the development of neuroinflammation. Paclitaxel can also lead to microglial and astrocyte activation and an increase in macrophage number in DRG and peripheral nerves. The release of cytokines stimulates the TLR4 receptor in DRG cells and blocking this receptor decreases pain behaviors in mice. IL-10 also can attenuate paclitaxel-induced CIPN. Inhibition of macrophages and microglia can also prevent the development of mechanical hyperalgesia and epidermal nerve fiber loss (Krukowski et al., J. Neurosci. 2016; 36:11074-11083; Zhang et al., J. Pain. 2012; 13:293-303; Ruiz-Medina et al., Eur. J. Pain. 2013; 17:75-85; Zhang et al., J. Pain. 2016; 17:775-786; Liu et al., Mol. Pain. 2010; 6:76; Li et al., J. Pain. 2014; 15:712-725). Still another potential contributor to paclitaxel-induced CIPN is activation of astrocytes and production of inflammatory cytokines, with effective treatment by minocycline (Zhang et al., J Pain. 2012 March; 13 (3): 293-303).

Another drug that disrupts microtubules and causes multiple toxicity reactions that include CIPN is vincristine, a member of the vinca alkaloid family. Part of the mechanism by which vincristine induces axonal neuropathy is by disrupting the microtubular axonal transport system. Vincristine binds with tubulin and blocks its polymerization into microtubules. As a result of this activity, vincristine inhibits axonal transport. Vincristine also causes axonal cytoskeletal changes of other sorts (Cioroiu et al., Curr. Neurol. Neurosci. Rep. 2017; 17:47). Vincristine induces distal axonal degeneration, and vincristine-induced peripheral neuropathy is associated with pain (Boyette-Davis et al., Pain Manag. 2018; 8:363-375). Symptoms of distal numbness and tingling commonly begin about 4-5 weeks after treatment. Vincristine-induced neuropathy tends to involve both motor and sensory fibers, with small fiber modalities and autonomic fibers particularly affected (Topp et al., J. Comp. Neurol. 2000; 424:563-576).

The family of platinum-based chemotherapeutic agents are also well known to cause multiple side effects, including CIPN. Acute and chronic neurotoxicity following platinum-based chemotherapy is a major challenge, and contributes to prolonged infusion times, dose reductions, treatment delays, and even the cessation of treatment (Storey et al., Ann. Oncol. 2010; 21:1657-1661). In addition to peripheral neuropathy, cisplatin may also induce ototoxicity, myelotoxicity and nephrotoxicity.

Cisplatin is one of the most widely prescribed chemotherapeutic drugs, and is prescribed in nearly 50% of all tumor chemotherapies. Cisplatin is used in treating a wide range of pediatric and adult malignances such as ovarian, testicular, bladder, head, neck, breast and lung. (Galanski et al., Curr. Med. Chem. 2005; 12:2075-2094). Cisplatin has multiple toxic side effects, with around 40 side effects reported thus far (Qi et al., Chem. Res. Toxicol. 2019; 32:1469-1486). Due to the use of extensive supportive medical care of cisplatin treated cancer patients, use of high-dose treatments have become more common, and include problems, such as acute kidney injury, GI problems including persistent diarrhea, neurological disorders, and loss of hearing. The many side effects of cisplatin lead to reduction or cessation of therapy or have a major impact on patients' quality of life, leading to higher levels of negative states such as depression and anxiety. (Crona et al., Oncologist. 2017; 22:609-619; Grunberg et al., Cancer Chemother. Pharmacol. 1989; 25:62-64; Van der Hoop et al., Cancer. 1990; 66:1697-1702; Perse Cisplatin Mouse Models: Treatment, Toxicity and Translatability Biomedicines. 2021 October; 9 (10): 1406). One of the toxic effects of cisplatin treatment is cisplatin-induced peripheral neuropathy (CisIPN), which occurs in a time- and dose-dependent manner. The development of CisIPN seems to be independent of pretreatment, age, sex, tumor type, and cotreatment with other chemotherapeutics (Park et al., Cancer J. Clin. 2013; 63:419-437; Schmoll et al., J. Clin. Oncol. 2003; 21:4083-4091).

Furthermore, oxaliplatin treatment can also result in oxaliplatin-induced CIPN. Risk factors for oxaliplatin-induced CIPN, which can present both acutely and chronically, include the cumulative oxaliplatin dose, the infusion time, low body weight, younger age, a body surface area>2,0, and several gene variations including variation in and voltage-gated sodium channel genes SCN4A, SCN9A and SCN10A) (Velasco et al., J. Neurol. Neurosurg. Psychiatry. 2014; 85:392-398; Alejandro et al., Am. J. Clin. Oncol. 2013; 36:331-337; Palugulla et al., Asian Pac. J. Cancer Prev. 2017; 18:3157-3165).

The antineoplastic mechanisms of platinum-based chemotherapeutic action, any or all of which may be relevant to the CIPN and other toxicities caused by exposure to these compounds include factors, such as binding to DNA and formation of DNA-platinum adducts, resulting in the inhibition of DNA replication and RNA (ribonucleic acid) transcription; activation of apoptosis pathways by the follow DNA adducts; disruption of mitochondrial function followed by the disruption of respiratory chain function and increased production of reactive oxygen species (ROS); inhibition of mitochondrial DNA replication and transcription, leading to an altered mitochondrial function and the activation of apoptosis; activation of the immune system (macrophages, T-cells and monocytes) followed by the release of pro-inflammatory cytokines and the activation of apoptosis; effects on calcium signaling pathways and the function of protein kinase families, which also can lead to apoptosis (Dasari et al., Eur. J. Pharmacol. 2014; 740:364-378; Tesniere et al., Oncogene. 2010; 29:482-491; Canta et al., Mitochondrial Dysfunction in Chemotherapy-Induced Peripheral Neuropathy (CIPN) Toxics. 2015; 3:198-223; Ray et al., J. Biomol. Struct. Dyn. 2018 doi: 10.1080/07391102.2018.1531059; Riddell Cisplatin and Oxaliplatin: Our Current Understanding of Their Actions. Met. Ions Life Sci. 2018 doi: 10.1515/9783110470734-007; Mckeage et al., Br. J. Cancer. 2001; 85:1219-1225; Sharawy et al., Exp. Toxicol. Pathol. 2015; 67:315-322; Kober et al., Mol Pain. 2018; 14:1-16; Jaggi et al., Toxicology. 2012; 291:1-9; Viatchenko-Karpinski et al., Mol. Pain. 2018; 14:1-11). Although it has been suggested that accumulation of platinum adducts in dorsal root ganglia and trigeminal ganglion neurons are particularly important in CIPN induced by platinum containing drugs, the breadth of other damaging effects on cell function, and the ability of inhibition of other processes to provide some indications of benefit suggest that other processes also need to be considered for their potential importance.

Still another class of anti-cancer agents known to cause CIPN and other types of damage to normal tissues are the protease inhibitors, of which bortezomib and carfilzomib are best studied. Peripheral neuropathy is seen in about one-third of patients treated with these drugs, and may last for weeks, months or even years after drug termination. Patients receiving bortezomib develop chronic, distal and symmetrical sensory peripheral neuropathy often accompanied by a neuropathic pain syndrome, with the potential of inflammation, myelin damage and other problems. (Saifee et al., J. Peripher. Nerv. Syst. 2010; 15:366-368; Peng et al., Support. Care Cancer. 2015; 23:2813-2824; Farquhar-Smith et al., Curr. Opin. Support. Palliat. Care. 2011; 5:1-7).

Thus, it is clear that there are multiple types of damage that need to be addressed in the context of damage by toxic insults, even in the limited situation of CIPN. Identification of therapeutic agents that are capable of addressing these multiple types of damage is a difficult scientific challenge, with few indications of how to successfully achieve such goals. The goal of addressing multiple types of damage contrasts with more standard goals of attempting to treat each type of damage individually, for example by attempts to identify therapies that target mitochondrial health and separate therapies that target repair of damage to myelin (as non-limiting examples).

Radiation-induced peripheral neuropathy (RIPN) also is a problem in cancer patients. RIPN is often a chronic handicap, progressive and usually irreversible, and often appears several years after radiotherapy. RIPN is rare when compared to CIPN but is increasing with improved long-term cancer survival. Technical progress in RT protocols has reduced its incidence in the last decades, but when it occurs it can greatly compromise the quality of life of the afflicted individual. (Delanian et al, Radiation-induced neuropathy in cancer survivors, 2012, 105 (3): 273-282). The pathophysiological mechanisms or RIPN are poorly understood. Nerve compression by indirect extensive radiation-induced fibrosis is thought to play a central role, but other potential contributors include axonal damage and demyelination and injury to blood vessels by ischaemia following capillary network failure. In general, RIPN is associated with delayed local damage to mature nerve tissue. Delayed effects enhance damage in the irradiated tissue and may include direct axonal injury and demyelination, extensive fibrosis within and surrounding nerve trunks, and ischemia by injury to capillary networks supplying the nerves compensated for by neovascularization (Delanian et al., Radiation-induced neuropathy in cancer survivors, 2012, 105 (3): 273-282).

RIPN is thought to partly be due to initial microvascular injury, followed by radiation-induced fibrosis (RIF) combined with specific neurological injury. RIF itself is a complex process with multiple components including fibroblast proliferation, extracellular matrix deposition, amplified by cytokines such as TGFβ1 and CTGF, along with inflammation, oxidative stress, damage to capillary networks and changes in fibroblast function. (Delanian et al., Radiother Oncol. 2004; 73:119-131; Denham et al., Radiother Oncol. 2002; 63:129-145) The acute phase post-irradiation also may show transient electrophysiological and biochemical changes combined with an altered vascular permeability in irradiated nerves (Pradat et al., Rev Neurol (Paris). 1994; 150:664-677).

Because radiation therapy is frequently applied in a localized manner, there is a great deal of attention given to relatively local types of nerve damage. For example, cranial nerve injury has been described after radiation therapy for intracranial and extracranial tumors. Radiation can also cause optic neuropathy, hypoglossal palsy, facial paralysis, and/or trigeminal neuropathy. When radiation is applied to the upper limb, then the treated individual may develop chronic brachial plexopathy, with a time to onset that ranges from several months to decades after treatment with a mean incidence of 1.8-2.9% per year, and early expression of RIBP is even more rare. With lower limb irradiation, radiation-induced fibrotic compression may contribute to nerve trunk damage.

The challenges in treating RIPN are that the many problems are generally delayed, which itself may cause problems in diagnosis due to a failure to consider the contributions of previous radiation therapy (Delanian et al., Radiation-induced neuropathy in cancer survivors, 2012,105 (3): 273-282). Treatments for RIPN are generally symptomatic and curative strategies are lacking. Symptomatic treatments include non-opioid analgesics, benzodiazepines, tricyclic antidepressants, anti-epileptics and membrane-stabilising drugs (e.g., carbamazepine). Surgical treatments were not useful, although physical therapy may be. Although pathogenesis of RIPN initially involves vascular mechanisms, fibrosis and atrophy are the main targets for therapeutic interventions. Combined pentoxifyllin-tocopherol (PE) significantly reduces radiation-induced fibrosis due to their synergistic clinical and biological properties (Delanian et al., J Clin Oncol. 2003; 21:2545-2550; Hamama et al., Radiother Oncol. 2012; 105:305-312).

Clodronate, a bisphosphonate, inhibits osteoclastic bone destruction with anti-inflammatory effects, and seems to inhibit macrophagic myelin nerve destruction in rats (Delanian et al., Semin Radiat Oncol. 2007; 17:99-107). Recently, clodronate, when combined with pentoxifylline-tocopherol (PENTOCLO), healed 54 patients with refractory osteoradionecrosis in a median of 9 months (Delanian et al., Int J Radiat Oncol Biol Phys. 2011; 80:832-839).

Damage to the nervous system is only one of the types of damage that can occur as a result of exposure to toxic agents, such as chemotherapy, irradiation, or industrial chemicals. Although damage to the nervous system is particularly well studied due to the important medical consequences of such damage, it is also critical to identify therapeutic strategies that can be used in the amelioration of damage caused to other tissues including, but not limited to, kidney, lung, immune system, digestive system, endocrine system, and other tissues as specified herein and in the literature and well known to those skilled in the arts.

For example, treatment for cancer with chemotherapeutic agents is well known to cause damage to the central nervous system and to be associated with both neurological and cognitive changes. This is a phenomenon that is often referred to as “chemobrain” and has been observed for multiple different kinds of cancers and multiple different types of chemotherapeutic agents. Changes in cognitive function can be observed during the course of treatment, and damage to the central nervous system can occur rapidly and continue for a long time. Damage to the central nervous system can involve multiple cell types, including myelin-producing oligodendrocytes, nerve cells, microglia endothelial cells and astrocytes. Astrocytes, as the major support cell in the brain, appear to be particularly sensitive to such damage and respond by upregulating one of their major cytoskeletal proteins called glial fibrillary acidic protein (GFAP). Such up regulation is considered to be a sign of astrocyte activation in response to injury.

Another example of an important and well-studied type of tissue damage caused by exposure to the toxic insults of chemotherapeutic agents is chemotherapy-induced nephrotoxicity (CINT). Damage can occur acutely, and can be lasting in duration (Santos et al., World J Clin Oncol. 2020, 24; 11 (4): 190-204; ncbi.nlm.nih.gov/pmc/articles/PMC7186234/; Rosner et al., N Engl J Med. 2017; 376:1770-1781; Perazella et al., Clin J Am Soc Nephrol. 2012; 7:1713-1721). CINT can be caused by multiple classes of anti-cancer treatments. As non-limiting examples, alkylating agents can cause acute kidney injury (AKI), hemorrhagic cystitis, inflammatory lesions, a syndrome of inappropriate antidiuretic hormone secretion (SIADH); and damage to proximal and distal tubular structures by action of metabolites and increased cellular oxidative stress. Antimetabolites can cause AKI, decreased glomerular filtration route (GFR), interstitial edema, and tubular acidosis. Anti-microtubular agents can cause SIADH, while antitumor antibiotics can cause nephrotic syndrome, focal segmental glomerular sclerosis, thrombotic microangiopathay (TMA), AKI and hemolytic uremic syndrome. Platinum agents can cause AKI, anemia hypomagnesemia and proximal tubular dysfunction (Rabah et al., Saudi J Biol Sci. 2010; 17:105-114; Carron et al., Hemodial Int. 2014; 18:846-847; Shavit et al., Kidney Int. 2014; 85:213; Cordonnier et al., Nephrologie. 1985; 6:19-26; Giroux et al., Am J Kidney Dis. 1985; 6:28-39; Bitran et al., Cancer. 1982; 49:1784-1788; Crona et al., Oncologist. 2017; 22:609-619; Raj et al., J Clin Oncol. 2006; 24:3095-3100).

As for CIPN, the causes of CINT are poorly understood, which makes the development of prophylactic or treatment strategies particularly challenging. (Santos et al., World J Clin Oncol. 2020 Apr. 24; 11 (4): 190-204; ncbi.nlm.nih.gov/pmc/articles/PMC7186234/). As with other types of toxicity caused by exposure to chemotherapeutic agents, a variety of disturbances of renal function have been reported and included in the general category of CINT (Santos et al., Nephrotoxicity in cancer treatment: An overview World J Clin Oncol. 2020 Apr. 24; 11 (4): 190-204; ncbi.nlm.nih.gov/pmc/articles/PMC7186234/). For example, treatments may cause AKI, due to toxic acute tubular necrosis, TMA, and crystal nephropathy; proteinuria/nephrotic syndrome due to TMA and glomerulopathies; tubulopathies due to electrolyte and acid-base disorders; and chronic kidney disease (CKD) due to glomerulopathies or interstitial nephritis). There may also be intrinsic kidney injury related to pre-existing patient risk factors mainly related to higher age, hypertension, diabetes, congestive heart failure, cirrhosis, hepatic failure, hyperbilirubinemia and hypoalbuminemia. There are also kidney-related risk factors, such as nephrosis, previous kidney injury, nephrotic syndrome and hydroelectrolytic disturbance due to vomiting, diarrhea and use of diuretics. (Perazella M A, Izzedine H. New drug toxicities in the onco-nephrology world. Kidney Int. 2015; 87:909-917).

One of the particularly dangerous side effects of treatment with chemotherapy can be the effects of these agents on the hematopoietic system. Chemotherapy-induced hematopoietic toxicity is a multifactorial challenge that affects the treatment of oncology patients. Of these problems, that of neutropenia (i.e., a fall in the number of neutrophils) is particularly dangerous because of its ability to increase the risk of infection. Neutropenia, however, is just one of several hematopoietic toxicities, which also include thrombocytopenia and anemia pubmed.ncbi.nlm.nih.gov/12166034/). Hemorrhage secondary to decreases in platelets is the major risk posed by chemotherapy-induced thrombocytopenia. The frequency of cancer-related anemia is dependent on the type, stage, and duration of disease. Chemotherapy-induced anemia is affected by the types of agents used, the schedule of drug administration, and the intensity of the regimen. Fatigue is the most common symptom of anemia, being reported by 80-100% of patients undergoing chemotherapy. Although fatigue is a major factor in patients' quality of life, it has often not been treated systematically and aggressively.

Despite extensive research on the side effects of treatment with chemotherapeutic agents, there are few agents that have proven even partially useful in preventing or reversing this damage, or in treating the symptoms created by such damage. Thus, while existing research has identified mechanisms of potential interest to consider, the success of translation of these ideas has been unsuccessful and therefore the prior research results cannot serve as a predictor of a medically useful outcome.

This general problem is illustrated by examination of attempts to develop treatments for CIPN and other types of damage to the nervous system. In this case there is not only extensive research on the effects of cancer treatment on the peripheral and central nervous system. CIPN also benefits from an ability to define outcome measures that can be determined non-invasively, and quantitative information often can be obtained by use of patient questionnaires. Given the high prevalence of CIPN among cancer patients, improvements in the management of several cancer types and increasing numbers of cancer survivors, discovering new, effective strategies to prevent and/or treat CIPN and their long-term consequences is considered a matter of urgency. The proportion of cancer patients who experience neuropathic pain at some point in their survivorship ranges from 40% to 70%. However, improved CIPN treatment is needed, with a recent systematic review demonstrating insufficient evidence to confirm the efficacy of central nervous system (CNS) drugs for CIPN. There are no known preventatives, and for many patients, control of the pain requires opioids.

This problem exists at a general level and exists even when one looks more specifically at single agents, as illustrated in a recent review on cisplatin-induced toxicities (Perše Cisplatin Mouse Models: Treatment, Toxicity and Translatability Biomedicines. 2021 October; 9 (10): 1406) in which the author concluded “There is no effective therapy for the prevention of these side effects; the current treatment strategy is symptomatic with limited effectiveness.” Thus, although there is widespread agreement that preventing CIPN is a highly desired goal, and multiple efforts have been made to find agents able to do this, success has been lacking (e.g., Hershman et al. Prevention and management of chemotherapy-induced peripheral neuropathy in survivors of adult cancers: American Society of Clinical Oncology clinical practice guideline. J. Clin. Oncol. 2014; 32:1941-1967; Hou et al Treatment of chemotherapy-induced peripheral neuropathy: systematic review and recommendations. Pain Physician. 2018; 21:571-592). Although some compounds showed initial promise, none are currently applied or recommended. As noted in a recent review on this topic (Zajączkowska et al Mechanisms of Chemotherapy-Induced Peripheral Neuropathy Int J Mol Sci. 2019 March; 20 (6): 1451), “Clinical practice guidelines promulgated by the American Society of Clinical Oncology (ASCO) do not recommend any agent for the prevention of CIPN. In the ASCO guidelines, for the treatment of established CIPN, a moderate recommendation was made for duloxetine. There was also a weak recommendation for a topical gel containing baclofen, amitriptyline and ketamine.”

Looking at this problem in greater detail, a Cochrane systematic review of interventions and an expert group systematic review by the American Society of Clinical Oncology (ASCO) recommended against the use of a wide range of interventions that have been tried in the field, indicated by research findings in the laboratory, or extrapolated from results in other fields. Therapeutic approaches recommended against in this review include acupuncture, cryotherapy, exercise therapy or ganglioside-monosialic acid (GM-1), retinoic acid, amifostine, amitriptyline, calcium magnesium infusion (Ca/Mg), calman-gafodipir, cannabinoids, carbamazepine, L-carnosine, diethyldithiocarbamate (DDTC), gabapentin, pregabalin, glutamate, glutathione, goshajinkigan (GJG), metformin minocycline, N-acetylcysteine, nimodipine, omega-3 fatty acids, ORG 2766, oxcarbazepine, recombinant human leukemia inhibitory factor, venlafaxine, vitamin B or vitamin E) in CIPN. Moreover, acetyl-L-carnitine is strongly advised against due to high-quality evidence indicating worsening neuropathy of neurotoxicity. For instance, alpha-lipoic acid, OPERA, curcumin, and Neuronorm have shown no benefit in randomized controlled trials despite positive findings in preclinical studies. Similarly, despite positive pilot studies, a large phase III trial did not demonstrate a significant neuroprotective effect of vitamin E and glutathione supplementation. Patients administered diethyldithiocarbamate (DDTC), with lower cumulative doses of cisplatin, also were more likely to withdraw from treatment due to CisPN-related adverse events. Similarly, the hexapeptide analogue of ACTH, ORG 2766, increased the incidence of CIPN in a smaller cohort study. Caution is advised with nutraceuticals and supplements with unproven efficacy. Thus, the prior studies were unsuccessful in predicting treatment outcomes.

Additionally, in two randomized, standard-of-care-controlled trials and a smaller non-randomized standard-of-care-controlled trial, glutamine was associated with reduced incidence and severity of dysaesthesias, nerve conduction impairment and interference with daily functioning. Amifostine demonstrated a clinically meaningful benefit for the prevention of sensory and auditory CIPN but was associated with worsening nausea and vomiting and thus is unlikely to be used clinically. There is also some evidence for efficacy for duloxetine in decreasing patient discomfort by symptom modification, but even here the average benefit is for an 0.72 point reduction in pain on a 0-10 point scale.

In a review of this problem by Majithia et al. in November 2016 (New Practical Approaches to Chemotherapy-Induced Neuropathic Pain: Prevention, Assessment, and Treatment. cancernetwork.com/view/new-practical-approaches-chemotherapy)-induced-neuropathic-pain-prevention-assessment-and-treatment), the authors also point out that one of the problems in identifying treatments for CIPN is that extrapolation from trials in patients with other neuropathic pain syndromes must be done with caution, since other syndromes sometimes respond to agents that CIPN does not respond to. Preventions and treatments of established CIPN both have been generally unsuccessful. Even though anticonvulsants, such as gabapentinoids and lamotrigine, were utilized for treatment of other neuropathic pain states, they have not been proven effective for patients with CIPN and did not change symptom severity any more than a placebo. The level of complexity revealed in analysis of CIPN has been a topic of particular interest due to its prevalence and its contribution to decreasing or stopping anti-cancer treatment. Nonetheless, the multiple other cellular and tissue targets affected by anti-cancer therapies are just as problematic and complex.

The novelty of the present invention is highlighted by the fact that none of the previous therapies of potential interest for ameliorating damage caused by chemotherapy and irradiation are potassium channel blockers. This is the case for CIPN, and is also the case for damage to other tissues.

Another example of toxic effects of treatment with cancer therapies, and the need for treatments that ameliorate such toxicities, involves renal damage of various sorts. Kidney injury, including both AKI and kidney injury with later onset, are common and serious complications of cancer and/or of its treatments. AKI is probably the most common form of renal dysfunction in cancer patient, and has multiple negative consequences. AKI also may disturb the bioavailability and/or safety profile of many oncological drugs, potentially leading to suboptimal treatments, or enhance the risk for drug-induced toxicities. Preventing kidney injury is widely recognized as a means of improving oncological outcomes and preventing unnecessary dose reductions or interruptions of potentially life-prolonging oncological treatments. There is also increased mortality in cancer patients who develop AKI on top of a pre-existing CKD, as compared with those without kidney disease.

Cytotoxic chemotherapy, targeted agents, as well as immune checkpoint inhibitors are often nephrotoxic. Among cytotoxic chemotherapeutic agents, the ones most commonly related to the development of AKI are cisplatin (CDDP), mitomycin-C (MM-C), gemcitabine, methotrexate (MTX), ifosfamide and pemetrexed. Paclitaxel also may cause kidney damage. Cisplatin-induced nephrotoxicity is particularly well-studied and appears to be multifactorial (Yao et al Cisplatin nephrotoxicity: a review. Am J Med Sci. 2007, 334:115-124). Cisplatin can cause massive oxidative stress injury and tubular cell apoptosis (Volarevic et al. Molecular mechanisms of cisplatin-induced nephrotoxicity: a balance on the knife edge between renoprotection and tumor toxicity. J Biomed Sci 2019; 26:25). It also can cause mitochondrial dysfunction, decreased ATPase activity, impaired solute transport and altered cation balance. As a consequence of such changes, sodium and water reabsorption is decreased, and salt and water excretion are increased, often leading to polyuria. Although renal function improves in most patients, some patients develop non-reversible renal impairment.

Preventive measures for chemotherapy-induced renal damage are limited. Hyper-hydration and forced diuresis (eventually with the use of mannitol) may decrease the incidence of AKI in patients receiving cisplatin. Intravenous (i.v.) magnesium supplementation (8-20 mEq) also may limit renal damage, although it is not clear if this is any better than adequate oral prehydration with diuresis. (Yamamoto et al. Hydration with 15 mEq magnesium is effective at reducing the risk for cisplatin-induced nephrotoxicity in patients receiving cisplatin (≥50 mg/m2) combination chemotherapy. Anticancer Res. 2016; 36:1873-1877; Saito et al. Premedication with intravenous magnesium has a protective effect against cisplatin-induced nephrotoxicity. Support Care Cancer 2017; 25:481-487).

The only drug approved by the FDA for protecting against cumulative nephrotoxicity is the ROS scavenger amifostine. Side effects, cost, and concerns that it also diminishes antitumor effect have, however limited the use of amifostine in clinical practice. Multiple natural compounds and drugs (e.g. allopurinol and statins) have been proposed to prevent cisplatin- and other cytotoxics-related nephrotoxicity, but the level of evidence for all of them appears to be low (Heidari-Soreshjani et al. Phytotherapy of nephrotoxicity-induced by cancer drugs: an updated review. J Nephropathol 2017; 6:254-263; Volarevic et al. Molecular mechanisms of cisplatin-induced nephrotoxicity: a balance on the knife edge between renoprotection and tumor toxicity. J Biomed Sci 2019; 26:25). In the case of gemcitabine, ifosfamide and pemetrexed, no measures to prevent AKI from these agents have been established to date, except those general interventions used to prevent AKI in non-oncological patients.

There is continued speculation about the pathogenesis of chemotherapy-related thrombotic microangiopathy (TMA), another aspect of kidney damage caused by cancer treatments. In some cases, microvascular thrombosis is the key event, but it is not clear whether this results from direct endothelial toxicity or from immune-mediated effects on other targets. TMA is also thought to be a common cause of late-onset AKI in patients who have undergone high-dose chemotherapy followed by hematopoietic stem cell transplantation (HSCT). The pathogenesis of TMA after HSCT is not well-understood, but damage to renal endothelial cells is thought to play a key role (Wanchoo et al. Acute kidney injury in hematopoietic stem cell transplantation. Curr Opin Crit Care 2019; 25:531-538).

Some of the most extensive efforts to discover means of preventing damage caused by exposure to toxic insults have focused on the problem of CIPN. There are many reasons for focusing on this topic, including the large numbers of people affected by this problem, its importance as a dose-limiting toxicity that can decrease the ability to treat cancer effectively, and the ability to study the evolution of CIPN in a relatively straightforward manner. The only other toxicity for which a similar degree of attention has been paid is chemotherapy-induced damage to the hematopoietic system. Attempts to develop therapies that overcome the effects of chemotherapy on the hematopoietic system have, however, been far more successful than has been the case for CIPN. The classification of CIPN as a peripheral neuropathy has led to a natural interest in determining whether any approaches that have shown promise in the treatment of peripheral nerve damage are helpful in the prevention or treatment of CIPN. Unfortunately, efforts in this regard have been thus far unsuccessful.

The approach of central interest to this invention, which is the application of potassium channel blockers, and preferentially 4-AP to the prevention and/or treatment of CIPN, has not been considered in respect to the problems on CIPN. Indeed, a detailed examination of findings on 4-AP revealed a significant body of evidence that teaches against the use of 4-AP even for the narrow purpose of preventing or treating CIPN. There are many reasons indicating that 4-AP was unlikely to be useful in preventing or treating CIPN or other peripheral neuropathies, let alone preventing other types of damage caused by exposure to toxic insults.

One of the early suggestions that 4-AP might have utility in pain management was reported in studies in individuals with chronic spinal cord injury. In these studies, 4-AP was reported to decrease neuropathic pain in some individuals (Hansebout et al., 4-Aminopyridine in chronic spinal cord injury: a controlled, double-blind, crossover study in eight patients. J. Neurotrauma 1993; 10 (1): 1-18). Despite multiple further studies on 4-AP, and the importance of decreasing chronic pain in individuals with spinal cord injuries, these studies did not develop into a treatment. Instead, Phase III studies on the effects of 4-AP in individuals with chronic spinal cord injury failed to reveal significant benefits.

As another example of the counter-indications for the hypothesis that 4-AP might be useful in treating CIPN, it was reported in 1988 that 4-AP can alter peripheral nerve conduction properties three months after induction of peripheral neuropathy by exposure to chronically large amounts of pyridoxine (Vitamin B6), a naturally occurring compound that has occasionally been used as to induce changes in peripheral nerve function (Bowe et al., 1988, Exp. Neurol., 100:448-458). Examination of the details of these studies reveals that they were conducted on nerves that were studied in ex vivo recording chambers at room temperature and were exposed to 4-AP at concentrations (1 mM) three orders of magnitude greater than the maximum serum concentrations that are considered clinically relevant. Moreover, all that was revealed were changes in sensitivity to 4-AP of nerves 3 months after pyridoxine treatment, no analyses of pain were conducted in these studies, and the paper contained no inferences in regards to the understanding or treatment of sensory neuropathy.

Another study revealed the ability of 4-AP to promote recovery from acute peripheral nerve traumatic crush injuries that were sufficiently precise in their location so as to allow treatment with localized application of a sustained release formulation of 4-AP, as well as by systemic treatment (Tseng et al. 4-Aminopyridine promotes functional recovery and remyelination in acute peripheral nerve injury. EMBO Mol Med (2016)8:1409-1420). Such injuries are qualitatively different from the diffuse injuries associated with systemic exposure to a chemotherapeutic agent or other toxic insults. Studies on using 4-AP to enhance recovery from localized peripheral nerve crush injuries also reported that treatment with 4-AP enhanced recovery on the specific parameter or thermal hyperalgesia, but not mechanical allodynia. In contrast, in the case of the present invention, 4-AP provided benefits in regards to paclitaxel-induced CIPN on mechanical allodynia, and thermal hyperalgesia, and thermal hypoalgesia, an unusual and unexpected outcome due to the opposite direction of the symptomatic changes in the response to temperature. Accordingly, the above described different outcomes reinforced the lack of predictability of present outcomes from prior results.

It also was suggested that electrical stimulation might provide benefits in individuals with CIPN (Smith et al., Pilot trial of a patient-specific cutaneous electrostimulation device (MC5-A Calmare®) for chemotherapy-induced peripheral neuropathy. J Pain Symptom Manage, 2010, 40:883-891). Despite the earlier suggestions that electrical stimulation might be useful in treatment of CIPN, however, more recent randomized, placebo-controlled, and double-blind study showed “no difference between active or placebo groups in terms of pain, numbness/tingling, frequency of symptoms or impact on daily life activities.” (Tonezzer et al. Effects of transcutaneous electrical nerve stimulation on chemotherapy-induced peripheral neuropathy symptoms (CIPN): a preliminary case-control study. J Phys Ther Sci. 2017 April; 29 (4): 685-692; ncbi.nlm.nih.gov/pmc/articles/PMC5430273/). These results suggest that transcutaneous electrical nerve stimulation applied in the frequency variation mode was not able to improve the symptoms of CIPN during chemotherapy cycles.

As another relevant example, Guillain-Barré patients treated with 4-AP vs placebo showed no difference in their pain scores (p=0.92) as rated by the McGill Pain Inventory questionnaire and the visual analog scale (Use of 4-amino pyridine for treatment of peripheral neuropathies (2003) U.S. Pat. No. 6,503,931). There were also no clinically significant changes seen in nerve conduction velocity. (Meythaler et al. Phase IIB Randomized Trial on the Use of 4-Aminopyridine in Guillain-Barré Syndrome. Arch Rehabil Res Clin Transl. 2021 June; 3 (2): 100123). Thus, these studies demonstrated no utility of 4-AP in pain treatment in respect even to the types of neuropathy that may manifest in individuals with Guillain-Barré Syndrome.

The present studies disclose a previously unknown and unsuspected method to prevent and/or treat damage caused by toxic insult, with the potassium channel blocker 4-AP. The present studies investigated repeatedly administering to an individual exposed to toxic insults a clinically relevant dosage of 4-AP. This treatment can be initiated before, during, or after the toxic insult. This treatment also has the unexpected effects of being able to prevent damage and being able to promote durable repair at functional and structural levels. This treatment also has the unexpected effect of rescuing mitochondria from chemotherapy-induced damage. Thus, the present studies provide additional novel discoveries of new uses of 4-AP in addressing unmet medical needs. The present studies demonstrated unexpected benefits that cannot be predicted from any prior observations. The present studies also demonstrated a new discovery that 4-AP treatment can be used to treat the effects of adverse exposure to toxic insults, which may include such examples as chemotherapy, radiation, and environmental toxicants.

Moreover, the present invention provides a novel treatment of any exposure to toxic substances. For example, the present invention provides a treatment of tissue damage caused by cancer treatment with chemotherapy and/or radiation. The present invention provides a treatment of exposure to human-generated toxicants, such as a treatment of paclitaxel (PTX)-induced CIPN or cisplatin-induced CIPN despite the different chemical structures and biological activities of these two toxic compounds. The present invention also provides a treatment for nephrotoxicity caused by chemotherapy, for mitochondrial damage caused by chemotherapy, and for effects on the central nervous system caused by chemotherapy.

In respect to the effects of chemotherapy, the present studies first focused on CIPN as a specific example of a general problem. The mechanisms of injury involved in CIPN, although largely unknown, are thought to be very different from traumatic injury both in respect to their mechanisms of injury and the potential breadth of damage created. Focusing on the example of paclitaxel-induced CIPN, this is a microtubule-stabilizing drug. Such an activity has no apparent relationship to any effects of traumatic injury. An additional difference is that the pathological changes that occur after traumatic injuries follow defined sequences involving such localized events as localized cell death, immune infiltration, localized scarring and other well defined pathological changes. In contrast, exposure to chemotherapy, radiation or environmental toxins generally does not cause as rapid pathological changes and also causes different pathological consequences that can take a much longer time to develop. Thus, the novelty of using 4-AP in the treatment of toxic insult damage lies in the cause of the damage (i.e., toxic insult) and the unexpected ability of 4-AP to be useful in treating such damage.

Thus, the present invention is based, in part, on the unexpected results that 4-AP effectively treated tissue damage caused by a toxic insult. Thus, the present invention relates, in part, compositions and methods for treating a subject exposed to a toxic insult, such as a toxic compound or exposure to toxic levels of radiation. The method can include administering to the subject a pharmaceutical composition comprising a potassium channel blocker, which is preferably 4-AP, a derivative thereof, or a combination thereof. In some embodiments, the damage caused by the toxic insult can be multi-site and/or multi-organ. In some embodiments, the method can include administering to the subject a therapeutically effective amount of a pharmaceutical composition comprising 4-AP, a derivative thereof, or a combination thereof. In some embodiments, the pharmaceutical composition can be formulated to provide sustained release of the 4-AP, a derivative of 4-AP, or a combination thereof. In certain embodiments, 4-AP or a derivative thereof can be represented by a structure according to Formula (I)

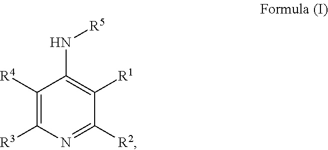

or an analog, a racemate, a tautomer, an isomer, an enantiomer, a diastereomer, a prodrug, a pharmaceutically acceptable salt, or a derivative thereof. In one embodiment, R¹, R², R³, R⁴, and R⁵ are each independently selected from hydrogen, halogen, C₁-C₆ alkyl, amine, hydroxyl, alkoxy, carboxyl, or any combination thereof; and wherein R¹, R², R³, R⁴, and R⁵ are optionally substituted. In some embodiments, the derivative of 4-AP comprises 3,4-diaminopyridine, 3-hydroxy-4-AP, or a combination thereof.

In some embodiments, 4-AP may be substituted with a different potassium channel blocker. It is known that 4-AP can bind to multiple potassium channels and that other potassium channel blockers, such as 4-AP derivatives and tetraethyl ammonium, can have similar effects in experimental situations and, in the case of the 4-AP derivative, 3,4-diaminopyridine, also in some clinical situations. Thus, it is anticipated in this invention that other potassium channel blockers may be also effective for the purposes for which 4-AP has been used in the experimental examples of this invention.

In some embodiments, the pharmaceutical composition can be administered to the subject by injection, intraperitoneal injection, intravenous injection, intramuscular injection, intrathecal injection, subcutaneous injection, sublingual administration, inhalation, oral administration, transdermal administration, implantation, insertion of a device into the subject, or any combination thereof.

In some embodiments, the pharmaceutical compositions can be administered in combination with an additional therapeutic agent in order to provide protection against the ability of at least one potassium channel blocker to cause convulsions in rare individuals if the serum levels exceed defined thresholds. For example, the pharmaceutical composition can be administered with an anticonvulsant. The anticonvulsant can be selected from lamotrigine, gabapentin, valproic acid, topiramate, famotodine, phenobarbital, diphenylhydantoin, phenytoin, mephenytoin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenytoin sodium, valproate sodium, clobazam, sulthiame, dilantin, diphenylan, and L-5-hydroxytrytophan, or any combination thereof.

The pharmaceutical compositions may be administered at various times, depending on the goal of treatment. In order to prevent effects of the toxic insult, the treatment may be administered prophylactically, beginning within the first week before or after exposure or before symptoms of the damage are apparent. In order to slow or reverse the progress of the damage, or to overcome the symptoms of the damage, treatment may be administered at any time when damage has begun to become apparent, as determined, for example, by the development of clinically relevant symptoms.

The pharmaceutical composition can be administered repeatedly throughout the duration of the exposure to the toxic insult if the goal is to prevent manifestation of injury. In some cases, and particularly when the treatment is being used to provide symptomatic relief, then treatment can be continued for as long as symptoms persist.

The subject in need of treatment can be administered a dose of from about 5 mg/day to about 100 mg/day of 4-AP, 4-AP derivative, or a combination thereof. In certain embodiments, the subject can be administered a dose of from about 5 mg/day to about 40 mg/day or about 40 mg/day to about 100 mg/day of 4-AP, 4-AP derivative, or a combination thereof. In some embodiments, the dosages of 4-AP or the derivative thereof may be increased or decreased from these levels depending on the effective therapeutic dosage range from each specific agent.

It will be also readily apparent to one skilled in the art that the quantity and frequency of the administration of the potassium channel blocker or the pharmaceutical composition thereof will depend on many factors including, but not limited to, the identity of the potassium channel blocker, type and severity of the subject's disease or disorder, condition of the subject, age of the subject, gender of the subject, overall health of the subject, and other factors, although appropriate dosages may be determined by clinical trials. Thus, the invention should not be construed to be limited to any particular dosage regime and the precise dosage and composition to be administered to any subject will be determined by the attending physical taking all other factors about the subject into account.

In some embodiments, the methods described herein can be used for treating damage caused by toxic insults in a subject. For example, the methods described herein can be used for treating the effects of exposure to chemotherapy or/and radiation therapy in an individual being treated for cancer. In some embodiments, the methods described herein can be used for treating the effects of exposure to industrial chemicals or toxic radiation from leakage at a nuclear plant or due to the release of toxic radiation in military scenarios. In other embodiments, the methods described herein can be used to treat the effects of exposure to environmental toxicants, for example as associated with air pollution, water pollution, exposure to chemicals released in burnpits, exposure to agricultural chemicals and other environmental exposures well known to those skilled in the arts.

In some embodiments, the damage can be most notable in one system of the body, such as in chemotherapy-induced peripheral neuropathy (CIPN). In other embodiments, the damage may be manifested in more than one tissue, including, for example, the peripheral nervous system, the central nervous system, the visual system, the auditory system, the hematopoietic system, the gastrointestinal system, the bladder, the heart, skeletal muscle, hair follicles, skin, vasculature, the salivary gland or a combination of any of these tissues.

In some embodiments, the toxic insult can be an industrial chemical used for any of a variety of purposes such as, for example, in agriculture and manufacturing processes, for which exposure causes damage to any of a variety of tissues including, for example, the peripheral nervous system, the central nervous system, the visual system, the auditory system, the hematopoietic system, the gastrointestinal system, the bladder, the heart, skeletal muscle, hair follicles, skin, vasculature, the salivary gland or a combination of any of these tissues. In the context of this invention, the specific focus is on agents that are produced intentionally by industrial processes and that have the capacity to cause tissue damage. Whether or not the agent that causes tissue damage was designed to be intentionally toxic, or whether it is accidentally toxic, does not matter. What is important is that the agent, be it a chemical structure or be it a form of electromagnetic radiation, is able to cause tissue damage and/or tissue dysfunction.

In some embodiments, the methods described herein can restore at least a portion of lost motor function or/and sensory function in the subject, enhance repair and regeneration of neural cells such as promote neural cell generation, enhance cell survival, reduce scarring, or combinations thereof, as compared to an untreated subject. In additional embodiments, the methods described herein can restore at least a portion of function of the damaged tissue, enhance repair and regeneration, enhance cell survival, reduce scarring, decrease other aspects of tissue damage or combinations thereof, as compared to an untreated subject. In further embodiments, the methods described herein can restore at least a portion of function of the damaged tissue, enhance repair and regeneration, enhance cell survival, reduce scarring, or combinations thereof, as compared to an untreated subject.

In some embodiments, the methods disclosed herein can be used for preventing or treating muscle atrophy. In some embodiments, the muscle atrophy can be due to exposure to chemotherapy, other toxic chemicals, toxic radiation.

In some embodiments, the methods disclosed herein can be used for preventing, ameliorating, reversing, or otherwise treating tissue dysfunction caused by exposure to toxic insults of human-generated origin. In some embodiments, the tissue dysfunction may occur in, for example, the hematopoietic system, hair follicles, cells in the mouth, digestive tract, reproductive system, and cells in the heart, kidneys, bladder, salivary glands, auditory system, visual system, lungs, nervous system, or any combination thereof.

In some embodiments, the methods disclosed herein can be used to identify individuals who will benefit from a treatment with potassium channel blockers (e.g., 4-AP, a derivative of 4-AP, or any combination thereof). For example, in some embodiments, the present invention relates to a method of identifying a subject responsive to a 4-AP administration to prevent, alleviate, or treat a tissue damage or tissue dysfunction caused by a toxic insult in a subject in need thereof, the method comprising the steps of: a) administering to the subject between 1 to 5 therapeutically effective amounts of a pharmaceutical composition comprising 4-AP, a derivative of 4-AP, or a combination thereof; b) evaluating the symptoms of the tissue damage or tissue dysfunction caused by the toxic insult in the subject; and c) identifying the subject as responsive to 4-AP administration to prevent, alleviate, or treat the tissue damage or tissue dysfunction caused by a toxic insult when the symptoms of the tissue damage or tissue dysfunction caused by the toxic insult in the subject improved.

In some embodiments, individuals manifesting dysfunction in one or more tissues following an exposure to toxic insults of human-generated origin may be treated with the methods disclosed herein for between one and ten days to determine if treatment provides improvement in tissue function. Thus, in some embodiments, the methods disclosed herein can be used to provide personalized targeting of therapies.

In some embodiments, individuals exposed to a toxic insult, and in whom one or more changes in tissue function caused by exposure to a toxic insult are present, are treated with between 1 to 5 treatments with a potassium channel blocker (e.g., 4-AP, derivative of 4-AP, or a combination thereof) in order to prognostically identify individuals in whom treatment should be continued for longer times. In some embodiments, if individuals show improvements in tissue function, then the long-term methods disclosed herein are applied. In some embodiments, an individual with damage caused by toxic insults and with symptoms in the realms of gait, pain, or nerve conduction velocity is treated with between 1 to 5 treatments of 4-AP or a derivative thereof, and the symptoms are measured between 1-8 hours after initiation of treatment (i.e., within two half-lives of 4-AP in the serum). For example, in some embodiments, an individual with CIPN or RIPN with symptoms in the realms of gait, pain, or nerve conduction velocity is treated with between 1 to 5 treatments of 4-AP or a derivative thereof, and the symptoms are measured between 1-8 hours after initiation of treatment (i.e., within two half-lives of 4-AP in the serum).

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “analog” or “analogue” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

The term “prodrug” refers to compounds that differ in structure from the reference molecule, but is chemically modified by a particular cellular process to ultimately become modified to retain the essential properties of the reference molecule or become the reference molecule. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. Examples of prodrugs include, but are not limited to, esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, sulfenyl thiocarbonates, optionally substituted sulfenyl thiocarbonates, 2-hydroxypropanoate ester, optionally substitute 2-hydroxypropanoate ester, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, sulfenyl, optionally substituted sulfenyl, sulfmyl, sulfonyl, sulfite, sulfate, sulfonamide, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally substituted hemiaminal, acetal, optionally substituted acetal, hemiacetal, optionally substituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, (acyloxybenzyl) ether, (acyloxybenzyl) ester, PEG ester, PEG carbonate, bis-(acyloxybenzyl) esters, optionally substituted bis-(acyloxybenzyl) esters, (acyloxybenzyl) esters, optionally substituted (acyloxybenzyl) esters, or BAB-esters, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

As used herein, the term “alkyl,” by itself or as part of another substituent means, unless otherwise stated, a straight or branched chain hydrocarbon having the number of carbon atoms designated (i.e. C1-6 means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, or —NO₂, preferably containing one or two substituents selected from halogen, —OH, alkoxy, —NH₂, trifluoromethyl, —N(CH₃)₂, or —C(═O)OH, more preferably selected from halogen, alkoxy, or —OH. Examples of substituted alkyls include, but are not limited to, 2,2-difluoropropyl, 2-carboxycyclopentyl, and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties.

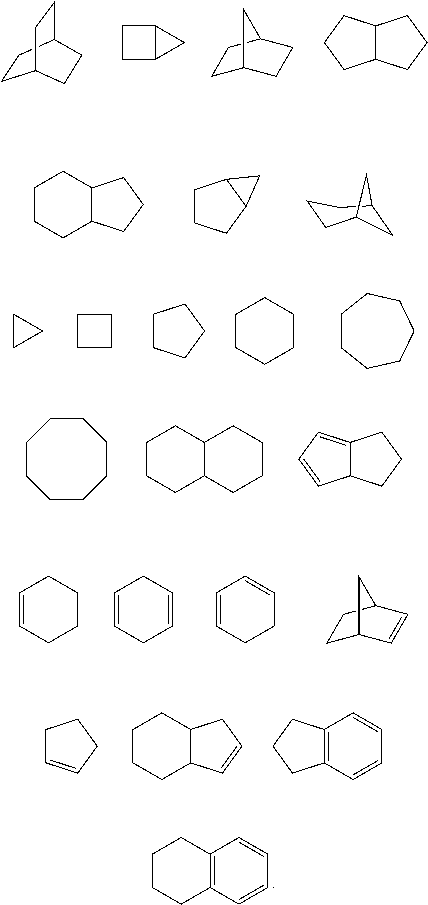

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heteroalkyl” by itself or in combination with another term means, unless otherwise stated, a stable straight or branched chain alkyl group consisting of the stated number of carbon atoms and one or two heteroatoms selected from O, N, Si, P, or S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from N, O, or S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

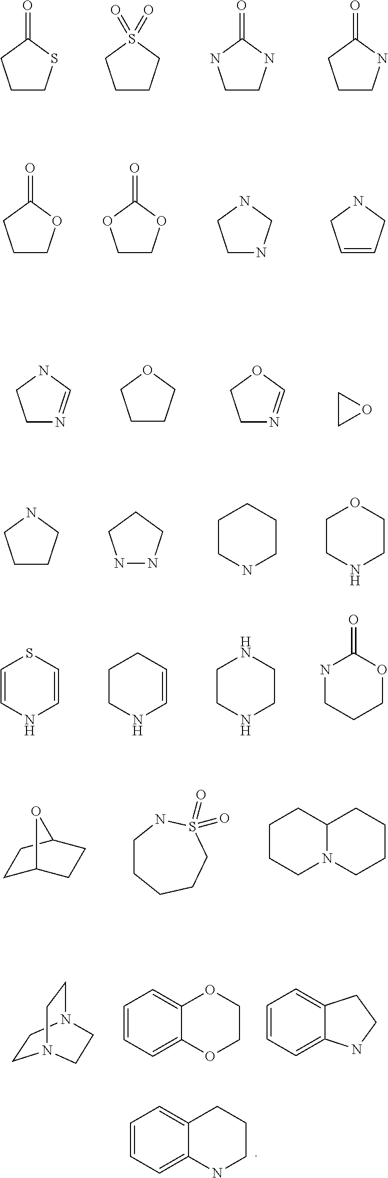

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aromatic” refers to a carbocycle or heterocycle with one or more polyunsaturated rings and having aromatic character, i.e. having (4n+2) delocalized p (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₄)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₄)alkyl” means an aryl-(C₁-C₄)alkyl functional group in which the aryl group is substituted. Preferred is substituted aryl(CH₂)—. Similarly, the term “heteroaryl-(C₁-C₄)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₄)alkyl” means a heteroaryl-(C₁-C₄)alkyl functional group in which the heteroaryl group is substituted. Preferred is substituted heteroaryl-(CH₂)—.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from C₁₋₆ alkyl, —OH, C₁₋₆ alkoxy, halo, amino, acetamido, or nitro. In yet another embodiment, the substituents are independently selected from C₁₋₆ alkyl, C₁₋₆ alkoxy, halo, acetamido, or nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]2, —OC(═O)N[substituted or unsubstituted alkyl]2, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]2, or —C(NH₂)[substituted or unsubstituted alkyl]2. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, or —C(═O)OH. In yet one embodiment, the substituents are independently selected from C₁₋₆ alkyl, —OH, C₁₋₆ alkoxy, halo, amino, acetamido, oxo, or nitro. In yet another embodiment, the substituents are independently selected from C₁₋₆ alkyl, C₁₋₆ alkoxy, halo, acetamido, or nitro. As used herein, where a substituent is an alkyl or alkoxy group, the carbon chain may be branched, straight or cyclic.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal that is compromised by biologically initiated processes. For example, such biological processes may be pathogens, such as viruses and bacteria. They can also be genetic diseases or autoimmune diseases caused by attack by the immune system on the cells of our own bodies.

The term “disorder” generally refers to any disturbance of normal functioning of the mind or body. For example, disorders are disruptions to normal health that are the subject of the current invention are not diseases, as defined in the above descriptions. This invention is focused on disruptions to normal health and tissue function that are specifically caused by exposure to toxic insults of human-made origin. Non-limiting examples of such toxic insults may be anti-cancer agents, industrial chemicals, radiation, and other disruptors of normal cell and tissue function. There is no single word that describes such disruptions. As with all disorders, there are biological consequences. In the case of disruptions caused by exposure to toxic insults of human-made origin, however, the biological changes follow on from the initial exposure to the toxic insult or insults.

The terms “cancer” or “neoplasm” as used herein, include, but are not limited to, benign and malignant cancers of the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, melanoma, etc.), breast, genital system, (e.g., uterus, ovary, prostate, testis, etc.), urinary system (e.g, bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

By the term “modulating,” as used herein, is meant mediating a detectable increase or decrease in the level of a tissue function or a response in a subject compared with the level of a tissue function or a response in the subject in the absence of a treatment or compound, and/or compared with the level of a tissue function or a response in an otherwise identical but untreated subject. The term encompasses perturbing and/or affecting a native signal or response thereby mediating a beneficial therapeutic response in a subject, preferably, a human.

A disease or disorder is “alleviated” if the severity of a sign or symptom of the disease or disorder, the frequency with which such a sign or symptom is experienced by a patient, or both, is reduced.

To “treat” a disease or disorder as the term is used herein, means to reduce the frequency or severity of at least one sign or symptom of a disease or disorder experienced by a subject.

The term “treating” refers to the administration of a therapeutically effective amount of a therapeutic agent (e.g., 4-AP) to a subject known or suspected to be exposed to toxic insults of human-made origin.

The term “treatment” refers to the medical management of a patient with the intent to cure, ameliorate, stabilize, or prevent a pathological condition, or disorder. This term includes active treatment, that is, treatment directed specifically toward the improvement of a pathological condition, or disorder. In general, it may include a causal treatment directed toward removal of the cause (i.e., the toxic insult or insults) of the associated pathological condition, or disorder. In contrast, treatment, as defined within this application, also includes preventative treatment, that is, treatment directed to minimizing or partially or completely inhibiting the development of damage that may be caused by exposure to toxic insults. Such treatments may be employed to supplement another specific therapy directed toward the durable improvement of the associated disease, pathological condition, or disorder.

As used herein, “treatment of disruptions to normal health and tissue function that are specifically caused by exposure to toxic insults of human-made origin” means reducing the severity and/or frequency with which a sign or symptom of the disruption to normal health and tissue function is experienced by a subject. Such treatments may include prevention of the disruption, stabilization of the disruption to slow or prevent progressive deterioration, reversal of the disruption, and/or partial or complete restoration of normal tissue structure and/or function. Treatments may also include management of symptoms of the tissue disruption to reduce their severity, with preventing, stabilizing or reversing dysfunction, and/or partially or completely restoring normal tissue structure and/or function.

“Treatment for prognostic purposes” refers to the use of the methods of the invention in order to identify individuals likely to benefit from continued treatment. Such treatments for prognostic purposes generally employ short-term treatment, for example (but not limited to) between one and five days of treatment and analysis of any of the symptoms of tissue dysfunction caused by exposure to a toxic insult or insults. In this way, responders to the treatment can be identified early so as to focus further attention on those individuals most likely to benefit from the treatments of this invention.

As used herein, the terms “therapy” or “therapeutic regimen” refer to those activities taken to alleviate or alter a disruption, or disruptions, to normal health and tissue function that are specifically caused by exposure to toxic insults of human-made origin, e.g., a course of treatment intended to reduce or eliminate at least one sign or symptom of such disruptions using pharmacological, surgical, dietary and/or other techniques. In general, a therapeutic regimen may include a prescribed dosage of one or more drugs or surgery. In the case of this invention, a therapeutic regimen may consist of treatment with 4-AP, a derivative thereof, or a different potassium channel blocker, or a combination of such agents. Such regimens may include other agents, such as anticonvulsants, to prevent possible side effects of 4-AP dosages in individuals of increased susceptibility to induction of seizures (which are not caused by dosages generally used in treatments with these agents). Therapies will most often be beneficial and reduce or eliminate at least one sign or symptom of the disruption of normal health and tissue function. In a more general use of the term “therapy”, some instances of the effect of a therapy will have non-desirable or side-effects as often occurs, e.g., with anti-cancer treatments. The effect of therapy will also be impacted by the physiological state of the subject, e.g., age, gender, genetics, weight, other disease conditions, etc.

A “therapeutic” treatment is a treatment administered to a subject who exhibits signs of pathology, for the purpose of diminishing or eliminating those signs.

The term “nephropathy” refers to the broad category of situations in which renal function is compromised in some way. As for neuropathy, the term nephropathy is used broadly to describe the symptomatic result of a variety of aspects of renal dysfunction, with no inferences as to the cause of such dysfunctions. Such tissue dysfunction may occur, for example, as a result of exposure to many different kinds of toxic insults of human-generated origin, with causes that may differ from nephropathies induced by such biological causes as kidney disease or ischemia-reperfusion injuries to a variety of different tissues, as may occur for example in myocardial infarctions. Nephropathies caused by exposure to toxic insults of human-generated origin represent another category of unmet medical need addressed by the present invention.

One example of damage caused by toxic insults is a collection of conditions that are included in the broad grouping of “peripheral neuropathies”. Peripheral neuropathies are a general description of a broad class of changes identified by symptoms presented, such as changes in sensation in peripheral nerves, but the underlying causes and mechanisms vary over a broad range. Thus, the term neuropathy describes symptoms, but includes a wide variety of different individual disruptions of normal tissue function. Thus, even though expression of some of the shared symptoms of peripheral neuropathies occurs in many different situations, there is little or no reason to believe that the underlying causes and pathologies are the same even for neuropathies caused by biological afflictions, such as diabetic neuropathy, neuropathic pain associated with spinal stenosis, peripheral neuropathy in autoimmune diseases, such as Guillain Barre Syndrome, neuropathic pain following spinal cord injury or stroke. The underlying causes and pathologies for neuropathies caused by toxic insults represent still a different broad category of afflictions that are united by the sharing of a symptom rather than by being caused by shared mechanisms or being treatable by shared approaches. The deficiency in using the designation of peripheral neuropathy as an indicator of a specific pathological process is even more so the case for neuropathic syndromes caused by exposure to toxic insults. There are many industrially-produced chemicals that can cause exposed individuals to exhibit symptoms that are described under the umbrella term of peripheral neuropathy, but expression of such symptoms has little or no bearing in understanding causation, pathophysiology or treatment of the clinical problem. Thus, it is understood that the term “peripheral neuropathy” is used only to indicate that a person expresses symptoms that would lead to their inclusion in this broad and multi-membered category, but that this term is not associated with specific causes or types of damage. There are many types of insults that can lead to outcomes that are collectively referred to as neuropathies, although that does not mean that the neuropathies are the same in respect to their detailed nature, their pathogenesis or their treatment.

Another example of damage associated with treatment with chemotherapy or irradiation is damage to the central nervous system. Such damage occurs in the treatment of a variety of different types of cancers regardless of whether or not the treatment is directed to the central nervous system. For example, patients treated for breast cancer frequently experience cognitive changes associated with a variety of different anti-cancer treatments, and equally frequently show signs of neurological changes as indicated by magnetic resonance imaging studies. As for other types of toxic responses that have been observed in cancer treatment, toxic responses in the brain also are lacking in therapeutic strategies.

Similar concerns as stated in the above two paragraphs apply to all disruptions of normal tissue function, regardless of the tissue in which they occur, which means that applying a particular category designation to a dysfunction generally reveals no information on either causation or remedy. As nonlimiting examples, dysfunctions of the visual system, auditory system, olfactory system, respiratory system, gastrointestinal system, genitourinary system, musculoskeletal system, peripheral nervous system, central nervous system, musculoskeletal system, and other parts of the body may be caused by many different means, and saying that a particular type of dysfunction exists in particular tissue generally reveals no information on either causation or remedy.

Thus, the observations that tissue dysfunction caused by exposure to toxic insults of human-generated origin may have characteristics that overlap with tissue dysfunction caused in other ways means that, as a general principle, the use of a similar term to place outcomes in a certain functional category has no implications as to cause, pathological underpinnings or treatment of the tissue dysfunction.

An “effective amount” or “pharmaceutically effective amount” of a compound is that amount of compound which is sufficient to provide a beneficial effect to the subject to which the compound is administered. An “effective amount” of a delivery vehicle is that amount sufficient to effectively bind or deliver a compound. The phrase “therapeutically effective amount,” as used herein, refers to an amount that is sufficient or effective to prevent or treat (delay or prevent the onset of, prevent the progression of, inhibit, decrease or reverse) a disruption to normal health and tissue function caused by exposure to toxic insults of human-made origin, including alleviating symptoms of such disruptions.

A “therapeutically effective amount” refers to that amount which provides a therapeutic effect for a given condition and administration regimen. In particular, “therapeutically effective amount” means an amount that is effective to prevent, alleviate or ameliorate symptoms of the disruption to normal health and/or tissue function that are caused by exposure to toxic insults of human-made origin, or prolong the survival of the subject being treated, which may be a human or non-human animal. Determination of a therapeutically effective amount is within the skill of the person skilled in the art. A “therapeutically effective amount” refers to that amount of a therapeutic agent that will have a durable beneficial effect, which may be curative, on the health and well-being of the subject with regard to a disruption to normal health and tissue function caused by exposure to toxic insults of human-made origin. The beneficial effect on the health and well-being of a subject can include, but is not limited to: (1) curing the condition; (2) slowing the progress of the condition; (3) causing the condition to retrogress; (4) decreasing the symptoms caused by exposure to the toxic insult. The beneficial effect on the health and well-being of a subject can also include prophylactic outcomes, including but not limited to: (1) preventing or delaying on-set of the damage to at least one tissue affected by exposure to the toxic insult; (2) maintaining the damage at a retrogressed level once such level has been achieved by a therapeutically effective amount of a substance; (3) preventing or delaying recurrence of the damage after a course of treatment; or, (4) decreasing the likelihood of tissue damage after exposure to the toxic insult or (5) decreasing any part of the symptoms caused by exposure to the toxic insult.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration. As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, topical, intraperitoneal, intramuscular, oral, aerosol, parenteral, ophthalmic, pulmonary, and topical administration. Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999); and oil-based delivery systems (Kirtane et al. 2022, Sci. Adv. 8, eabm8478).

The term “pharmaceutically acceptable” or “pharmacologically acceptable” can mean, but is in no way limited to, entities and compositions that do not produce an adverse, allergic or other untoward reaction when administered to an animal, or a human, as appropriate. “Pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected results that 4-aminopyridine effectively treated tissue damage caused by toxic insult. Thus, the present invention relates, in part, to compositions and methods for treating a subject exposed to a toxic insult, such as a toxic compound or exposure to toxic levels of radiation. The method can include administering to the subject a pharmaceutical composition comprising a potassium channel blocker. In some embodiments, the damage caused by the toxic insult can be multi-site and/or multi-organ. In some embodiments, the method can include administering to the subject a therapeutically effective amount of a pharmaceutical composition comprising 4-aminopyridine, a derivative thereof, or a combination thereof. In some embodiments, the pharmaceutical composition can be formulated to provide sustained release of the 4-aminopyridine, derivative of 4-aminopyridine, or a combination thereof. In certain embodiments, 4-aminopyridine or a derivative thereof can be represented by a structure according to Formula (I)

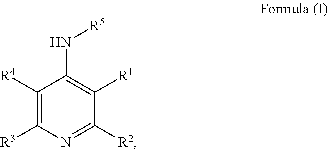

or an analog, a racemate, a tautomer, an isomer, an enantiomer, a diastereomer, a prodrug, a pharmaceutically acceptable salt, or a derivative thereof. In one embodiment, R¹, R², R³, R⁴, and R⁵ are each independently selected from hydrogen, halogen, C₁-C₆ alkyl, amine, hydroxyl, alkoxy, carboxyl, or any combination thereof, and wherein R¹, R², R³, R⁴, and R⁵ are optionally substituted. In some embodiments, the derivative of 4-aminopyridine comprises 3,4-diaminopyridine, 3-hydroxy-4-aminopyridine, or a combination thereof.

In some embodiments, the methods described herein can be used for treating damage caused by toxic insults in a subject. For example, the methods described herein can be used for treating the effects of exposure to chemotherapy or other chemical cancer treatments and/or radiation therapy in an individual being treated for cancer. In some embodiments, the methods described herein can be used for treating the effects of exposure to industrial chemicals, chemicals generated from different chemicals via chemical reactions (as may occur in burn pits, for example) or toxic radiation from leakage at a nuclear plant or due to the release of toxic radiation in military scenarios.

In some embodiments, the damage can be most notable in one system of the body, such as in chemotherapy-induced peripheral neuropathy. In other embodiments, the damage may be manifested in more than one tissue, including, for example, the peripheral nervous system, the central nervous system, the visual system, the auditory system, the hematopoietic system, the gastrointestinal system, the bladder, the genitourinary system, the heart, skeletal muscle, hair follicles, skin, vasculature, the salivary gland or a combination of any of these tissues.

In some embodiments, the methods described herein can restore at least a portion of lost motor function or/and sensory function in the subject, enhance repair and regeneration of neural cells such as promote neural cell generation, enhance cell survival, reduce scarring, or combinations thereof, as compared to an untreated subject. In additional embodiments, the methods described herein can restore at least a portion of function of the damaged tissue, enhance repair and regeneration, enhance cell survival, reduce scarring, decrease other aspects of tissue damage or combinations thereof, as compared to an untreated subject. In further embodiments, the methods described herein can restore at least a portion of function of the damaged tissue, enhance repair and regeneration, enhance cell survival, reduce scarring, or combinations thereof, as compared to an untreated subject.

In some embodiments, the methods disclosed herein can be used for preventing or treating muscle atrophy. In some embodiments, the muscle atrophy can be due to treatment with chemotherapy, other toxic chemicals, toxic radiation.

In other embodiments, the methods disclosed herein can be used for preventing or treating dysfunction in the peripheral nervous system, the central nervous system, the visual system, the auditory system, the hematopoietic system, the gastrointestinal system, the bladder, the genitourinary system, the heart, skeletal muscle, hair follicles, skin, vasculature, the salivary gland, or any combination thereof. In some embodiments, the tissue dysfunction can be due to treatment with chemotherapy, or exposure to other toxic chemicals, or toxic radiation.

### Compounds and Compositions

In one aspect, the present invention provides compounds effective in preventing, alleviating, and/or treating a tissue damage caused by a toxic insult, tissue dysfunction caused by a toxic insult, mitochondrial dysfunction caused by a toxic insult, muscle atrophy caused by a toxic insult, neuropathy caused by toxic insult, or other types of dysfunctions in any other aspect of the body, or any combination thereof. As nonlimiting examples, such dysfunctions may include dysfunctions of the visual system, auditory system, olfactory system, respiratory system, gastrointestinal system, genitourinary system, musculoskeletal system, peripheral nervous system, central nervous system, musculoskeletal system, and other parts of the body may be caused by many different means, and saying that a particular type dysfunction exists in particular tissue.

In one aspect, the present invention provides compounds effective in reducing or reversing a tissue damage caused by a toxic insult or insults, oxidation damage caused by a toxic insult or insults, and/or scarring caused by a toxic insult or insults. In one aspect, the present invention provides compounds effective in restoring, improving, and/or enhancing at least a portion of tissue function effected by toxic insult, myelination effected by toxic insult, tissue regeneration effected by toxic insult, cell survival effected by toxic insult, cell generation effected by toxic insult, repair or regeneration of endogenous stem or precursor cells effected by toxic insult, repair or regeneration of transplanted stem or precursor cells effected by toxic insult, repair or regeneration of stem or progenitor cells effected by toxic insult, or any combination thereof. In one aspect, the present invention provides compounds effective in reducing the lesion size effected by toxic insult, or any combination thereof. In one aspect, the present invention provides compounds effective in inhibiting at least one ion channel that is affected by 4-AP or a derivative thereof.

Thus, in some embodiments, the compound is a potassium channel blocker or an analog, a racemate, a tautomer, an isomer, an enantiomer, a diastereomer, a prodrug, a pharmaceutically acceptable salt, or a derivative thereof having the structure of Formula (I)

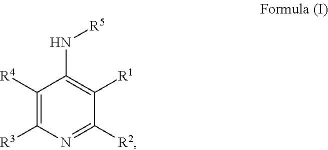

or an analog, a racemate, a tautomer, an isomer, an enantiomer, a diastereomer, a prodrug, a pharmaceutically acceptable salt, or a derivative thereof.

In various embodiments, R¹ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R¹ is optionally substituted. For example, in some embodiments, R¹ is hydrogen, halogen, C₁-C₆ alkyl, amine, hydroxyl, alkoxy, carboxyl, or any combination thereof.

In various embodiments, R² is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R² is optionally substituted. For example, in some embodiments, R² is hydrogen, halogen, C₁-C₆ alkyl, amine, hydroxyl, alkoxy, carboxyl, or any combination thereof.

In various embodiments, R³ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R³ is optionally substituted. For example, in some embodiments, R³ is hydrogen, halogen, C₁-C₆ alkyl, amine, hydroxyl, alkoxy, carboxyl, or any combination thereof.

In various embodiments, R⁴ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R⁴ is optionally substituted. For example, in some embodiments, R⁴ is hydrogen, halogen, C₁-C₆ alkyl, amine, hydroxyl, alkoxy, carboxyl, or any combination thereof.

In various embodiments, R⁵ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R⁵ is optionally substituted. For example, in some embodiments, R⁵ is hydrogen, halogen, C₁-C₆ alkyl, amine, hydroxyl, alkoxy, carboxyl, or any combination thereof.

For example, in one embodiment, the compound represented by Formula (I) is selected from 4-AP, or an analog, a racemate, a tautomer, an isomer, an enantiomer, a diastereomer, a prodrug, a pharmaceutically acceptable salt, or a derivative thereof, 3,4-diaminopyridine, or an analog, a racemate, a tautomer, an isomer, an enantiomer, a diastereomer, a prodrug, a pharmaceutically acceptable salt, or a derivative thereof, 3-hydroxy-4-aminopyridine, or an analog, a racemate, a tautomer, an isomer, an enantiomer, a diastereomer, a prodrug, a pharmaceutically acceptable salt, or a derivative thereof, N-(4-pyridyl)-t-butyl carbamate, or an analog, a racemate, a tautomer, an isomer, an enantiomer, a diastereomer, a prodrug, a pharmaceutically acceptable salt, or a derivative thereof, N-(4-pyridyl) ethyl carbamate, or an analog, a racemate, a tautomer, an isomer, an enantiomer, a diastereomer, a prodrug, a pharmaceutically acceptable salt, or a derivative thereof, N-(4-pyridyl) methyl carbamate, or an analog, a racemate, a tautomer, an isomer, an enantiomer, a diastereomer, a prodrug, a pharmaceutically acceptable salt, or a derivative thereof, N-(4-pyridyl) isopropyl carbamate, or an analog, a racemate, a tautomer, an isomer, an enantiomer, a diastereomer, a prodrug, a pharmaceutically acceptable salt, or a derivative thereof, or any combination thereof.

The compounds described herein may form salts with acids or bases, and such salts are included in the present invention. The term “salts” embraces addition salts of free acids or free bases that are compounds of the invention.

In one aspect, the present invention relates, in part, to compositions comprising one or more compounds of the present invention. In some embodiments, the composition comprises one or more compounds having the structure of Formula (I), or an analog, a racemate, a tautomer, an isomer, an enantiomer, a diastereomer, a prodrug, a pharmaceutically acceptable salt, or a derivative thereof. In some embodiments, the composition is the pharmaceutical composition. Thus, in one aspect, the present invention provides compositions effective in preventing, alleviating, and/or treating a tissue damage caused by a toxic insult, tissue dysfunction caused by a toxic insult, mitochondrial dysfunction caused by a toxic insult, muscle atrophy caused by a toxic insult, neuropathy caused by toxic insult, or any combination thereof.

In one aspect, the present invention provides compositions effective in reducing or reversing a tissue damage or dysfunction caused by toxic insult, oxidation damage caused by toxic insult, and/or scarring caused by toxic insult. In one aspect, the present invention provides compositions effective in restoring, improving, and/or enhancing at least a portion of tissue function effected by toxic insult, myelination effected by toxic insult, tissue regeneration effected by toxic insult, cell survival effected by toxic insult, neural cell generation effected by toxic insult, repair or regeneration of endogenous stem or precursor cells effected by toxic insult, repair or regeneration of transplanted stem or precursor cells effected by toxic insult, repair or regeneration of stem or progenitor cells effected by toxic insult, or other types of dysfunctions in any other aspect of the body, or any combination thereof. As non-limiting examples, such dysfunctions may include dysfunctions of the visual system, auditory system, olfactory system, respiratory system, gastrointestinal system, genitourinary system, musculoskeletal system, peripheral nervous system, central nervous system, musculoskeletal system, and/or other parts of the body. Tissue dysfunction may be caused by a variety of different means. Moreover, saying that a particular type of dysfunction exists in any particular tissue does not distinguish between the different types of damage that may lead to symptomatically similar outcomes, much as is the case for peripheral neuropathy. In one aspect, the present invention also provides compositions effective in reducing the lesion size effected by toxic insult.

In some embodiments, the toxic insult is an acute toxic insult, chronic toxic insult, or a combination thereof. Examples of such toxic insults include, but are not limited to, a non-biological substance, such as chemotherapy drugs, a toxic non-biological substance of natural origin (e.g., arsenic, lead, mercury), non-naturally occurring compound, toxin, environmental toxicant, agent used in treating cancer (e.g., chemotherapy agent), biological response modifier, toxic industrial chemical (e.g., chemicals used in manufacturing or agricultural settings), radiation (e g., cancer treatment, industrial accidents or military exposures), toxic radiation of natural origin (e.g., damaging amounts of heat produced by exposure to fire, extreme weather conditions, and/or sunlight-associated ultraviolet radiation sufficient to cause tissue damage), or any combination thereof.

In various embodiments the toxic insult is an anti-cancer agent. The anti-cancer agent can be any anti-cancer agent known in the art. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but are not limited to, chemotherapeutic agents, antiproliferative agents, anti-tumor agents, checkpoint inhibitors, and anti-angiogenic agents. For example, in one embodiment, the anti-cancer agent is gemcitabine, doxorubicin, 5-FU, tyrosine kinase inhibitors, sorafenib, trametinib, rapamycin, fulvestrant, ezalutamide, or paclitaxel.

Examples of chemotherapeutic agents include, but are not limited to, cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium), or any combination thereof.

Antiproliferative agents are compounds that decrease the proliferation of cells. Examples of antiproliferative agents include, but are not limited to, alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), levamisole, gallium nitrate, granisetron, sargramostim strontium-89 chloride, filgrastim, pilocarpine, dexrazoxane, ondansetron, or any combination thereof.

Examples of anti-tumor agents include, but are not limited to, cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Examples of anti-angiogenic agents include, but are not limited to, anti-VEGF antibodies, including humanized and chimeric antibodies, anti-VEGF aptamers, antisense oligonucleotides, angiostatin, endostatin, interferons, interleukin 1 (including alpha and beta) interleukin 12, retinoic acid, and tissue inhibitors of metalloproteinase-1 and -2. (TIMP-1 and -2), small molecules, including topoisomerases such as razoxane, a topoisomerase II inhibitor with anti-angiogenic activity, or any combination thereof.

Examples of other anti-cancer agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer, or any combination thereof.

In some embodiments, the anti-cancer agent may be a prodrug form of an anti-cancer agent. In certain embodiments, an anti-cancer agent may be chemically modified with an alkyl or acyl group or some form of lipid.

For example, in some embodiments, the toxic insult is a platinum-based antineoplastic agent, vinca alkaloid agent, epothilone agent, taxane agent, proteasome inhibitor, immunomodulatory drug, taxane, cisplatin, radioactive cancer treatment, exposure to damaging levels of radiation along the electromagnetic spectrum, nuclear energy accident, and nuclear warfare, environmental toxicant, or any combination thereof.

In one embodiment, the toxic insult is a hydrophobic agent. In one embodiment, the toxic insult is a hydrophilic agent. Examples of such toxic insults include, but are not limited to, one or more drugs, antibiotics, small molecules, anti-cancer agents, chemotherapeutic agents, immunomodulatory agents, gene-silencing agents, medical imaging agents, therapeutic moieties, poorly water soluble drugs, anti-cancer drugs, antibiotics, analgesics, anticonvulsants; anti-diabetic agents, antifungal agents, antineoplastic agents, anti-parkinsonian agents, anti-rheumatic agents, biological response modifiers, cardiovascular agents, contrast agents, diagnostic agents, gastrointestinal agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In some embodiments, the toxic agent may be an industrial chemical. Toxic industrial chemicals are found in a wide variety of circumstances, including manufacturing, agricultural use, packaging, food additives, and many other situations. The rate at which new chemicals are being generated every day and the importance of developing treatments for toxicity from the wide range of different structures of chemicals stresses the importance of developing therapeutic strategies for ameliorating such toxicities. The unexpected potency of 4-AP in preventing toxicity from both paclitaxel and cisplatin (which are very different chemical structures, and for which cisplatin is chemically more akin to an industrial chemical than to the taxanes that are industrially-generated formulations and derivatives of chemicals found in nature), and of being able to prevent toxicity to the peripheral nervous system, the central nervous system, the kidney, and to mitochondria indicate that 4-AP is likely to be useful in multiple circumstances beyond those defined in the experimental examples provided within the instant invention.

In some embodiments, the composition further comprises one or more therapeutic agent. In some embodiments, the therapeutic agent is any potassium channel blocker known in the art. Examples of such potassium channel blockers include, but are not limited to, bretylium, clofilium, dalfampridine, dofetilide, E-4031, ebastine, gliclazide, ibutilide, nifekalant, sematilide, sotalol, sulfonylureas, tedisamil, or any combination thereof.

In some embodiments, the composition further comprises one or more anticonvulsant agents. Examples of such anticonvulsant agents include, but are not limited to, barbiturate, benzodiazepine, bromide, carbamate, carboxamide, fatty acid, fructose or a derivative thereof, y-aminobutyric acid (GABA) or an analog thereof, hydantoin, oxazolidinedione, proprionate, pyrimidinedione, pyrrolidine, succinimide, sulfonamide, triazine, urea, valproylamide, or any combination thereof.

### Combinations

In one embodiment, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in one embodiment, the composition comprises a 1:1 ratio of two individual agents. However, the combination is not limited to any particular ratio. Rather any ratio that is shown to be effective is encompassed.

### Methods of Use

In one aspect, the invention provides methods of preventing, alleviating, and/or treating a tissue damage caused by a toxic insult, tissue dysfunction caused by a toxic insult, mitochondrial dysfunction caused by a toxic insult, muscle atrophy caused by a toxic insult, neuropathy caused by toxic insult, nephropathy caused by a toxic insult, or any combination thereof.

In one aspect, the present invention provides methods of reducing or reversing a tissue damage caused by toxic insult, oxidation damage caused by toxic insult, and/or scarring caused by toxic insult.

In some embodiments, the tissue damage is a multi-tissue damage, multi-organ tissue damage, or any combination thereof. In some embodiments, the tissue damage is a kidney tissue damage, liver tissue damage, heart tissue damage, lung tissue damage, brain tissue damage, central nervous system damage, peripheral nerve tissue damage, peripheral neuropathy, nephropathy, chemotherapy-induced peripheral neuropathy (CIPN), radiation-induced peripheral neuropathy (RIPN), chemotherapy-induced nephrotoxicity (CINT), neutropenia, gastrointestinal tract tissue damage, gut tissue damage, visual system tissue damage, auditory system tissue damage, skin tissue damage, bladder tissue damage, reproductive system tissue damage, hematopoietic system tissue damage, or any combination thereof. For example, in some embodiments, the CIPN is CIPN caused by taxane treatment (P-CIPN), CIPN caused by cisplatin treatment (CisTPN), or any combination thereof.

In some embodiments, the tissue dysfunction is a motor dysfunction, sensory dysfunction, cognitive dysfunction, visual dysfunction, auditory dysfunction, kidney dysfunction, hematopoietic system dysfunction, normal skin function, salivary gland dysfunction, liver dysfunction, gall bladder dysfunction, gastrointestinal (GI) dysfunction, sexual dysfunction, or any combination thereof.

In one aspect, the present invention provides methods of restoring, improving, and/or enhancing at least a portion of tissue function effected by toxic insult, myelination effected by toxic insult, tissue regeneration effected by toxic insult, cell survival effected by toxic insult, neural cell generation effected by toxic insult, repair or regeneration of endogenous stem cells effected by toxic insult, repair or regeneration of transplanted stem cells effected by toxic insult, repair or regeneration of progenitor cells effected by toxic insult, or any combination thereof.

In some embodiments, the tissue function is a motor function, sensory function, cognitive function, visual function, auditory function, kidney function, hematopoietic system function, normal skin function, salivary gland function, liver function, gall bladder function, gastrointestinal (GI) function, sexual function, or any combination thereof. In some embodiments, the tissue is a kidney tissue, liver tissue, heart tissue, lung tissue, brain tissue, central nervous system tissue, peripheral nerve tissue, gastrointestinal tract tissue, gut tissue, visual system tissue, auditory system tissue, skin tissue, bladder tissue, reproductive system tissue, hematopoietic system tissue, musculoskeletal tissue, or any combination thereof.

In one aspect, the present invention provides methods of reducing the lesion size effected by toxic insult.

In one aspect, the present invention provides methods of inhibiting at least one potassium channel blocker for the purpose of treating tissue damage caused by exposure to a toxic insult or insults.

In some embodiments, the method comprises administering to the subject an effective amount of a composition comprising at least one compound (e.g., at least one compound of Formula (I)) or composition of the present invention.

In one aspect, the present invention provides methods comprising administering at least one compound of the present invention or a composition thereof to the subject, wherein the subject was exposed to and/or affected by at least one toxic insult. The present invention also provides methods comprising administering at least one compound of the present invention or a composition thereof to the subject, wherein the subject has cancer.

In some aspects, the present invention provides methods of administering an effective amount of any compound or pharmaceutical composition disclosed herein to the subject. Thus, in some aspects, the present invention also provides methods comprising administering an effective amount of any compound or pharmaceutical composition disclosed herein to the subject, wherein the subject was exposed to and/or affected by at least one toxic insult. In some aspects, the present invention also provides methods comprising administering an effective amount of any compound or pharmaceutical composition disclosed herein to the subject, wherein the subject has cancer.

In various embodiments, the toxic insult is any toxic insult described herein. For example, in some embodiments, the toxic insult is a platinum-based antineoplastic agent, vinca alkaloid agent, epothilone agent, taxane agent, proteasome inhibitor, and immunomodulatory drug, taxane, cisplatin, radioactive cancer treatment, exposure to damaging levels of radiation along the electromagnetic spectrum, nuclear energy accident, and nuclear warfare, environmental toxicant, or any combination thereof.

In some embodiments, the treatment can be started at the time of the initiating insult, during continued exposure to toxic insult, or in the treatment of tissue damage that develops with a delay after the exposure to the toxic insult has ended.

The composition of the invention may be administered to a patient or subject in need systematically, locally, or a combination thereof.

The composition of the invention may be administered to a patient or subject in need in a wide variety of ways, including by inhalation, such as aerosol inhalation, injection, ingestion, oral administration, transdermal administration, transfusion, implantation, sublingual administration, or transplantation. The compositions described herein may be administered to a patient subcutaneously, intradermally, intratumorally, intranodally, intramedullary, intramuscularly, intrathecally, intravenously (i.v.), or intraperitoneally. In one embodiment, the composition is administered systemically to the subject. In one embodiment, the compositions of the present invention are administered to a patient by i.v. injection. In one embodiment, the composition is administered locally to the subject. In one embodiment, the compositions of the present invention are administered to a patient topically. Any administration may be a single application of a composition of invention or multiple applications. Administrations may be to single site or to more than one site in the individual to be treated. Multiple administrations may occur essentially at the same time or separated in time.

Subjects to which administration of the pharmaceutical compositions of the invention is contemplated include, but are not limited to, humans and other primates, mammals including but not limited to non-human mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

### Formulations/Pharmaceutical Compositions

The invention also encompasses the use of pharmaceutical compositions comprising a compound of the invention or a composition thereof. Such a pharmaceutical composition may comprise of at least one compound of the invention in a form suitable for administration to a subject, or the pharmaceutical composition may comprise at least one compound of the invention and one or more pharmaceutically acceptable carriers, one or more additional ingredients, or some combination of these. The compound of the invention may be present in the pharmaceutical composition in the form of a physiologically acceptable salt, such as in combination with a physiologically acceptable cation or anion, as is well known in the art.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

Depending on the intended mode of administration, the pharmaceutical composition can be in the form of, for example, solids, semi-solids, liquids, solutions, suspensions (e.g., incorporated into microparticles, liposomes, etc.), emulsions, gels, or the like, preferably in unit dosage form suitable for single administration of a precise dosage. The pharmaceutical compositions can include, as noted above, an effective amount of the potassium channel blocker such as 4-aminopyridine compound, a derivative thereof, or a combination thereof, in combination with a pharmaceutically acceptable carrier and, in addition, can include other carriers, adjuvants, diluents, thickeners, buffers, preservatives, surfactants, etc. Pharmaceutical compositions can also include one or more additional active ingredients such as other medicinal agents, pharmaceutical agents, antimicrobial agents, anti-inflammatory agents, anesthetics, anti-convulsants, and the like.

The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

The therapeutic and prophylactic methods of the invention thus encompass the use of pharmaceutical compositions comprising at least one compound of the invention, to practice the methods of the invention. The pharmaceutical compositions useful for practicing the invention may be administered to deliver a dose of from 0.001 ng/kg/day and 100 mg/kg/day. For example, in some embodiments, the pharmaceutical compositions useful for practicing the invention may be administered to deliver a dose of from 0.005 mg/kg/day and 5 mg/kg/day. In one embodiment, the invention comprises administration of a dose which results in a concentration of the compound of the present invention from 10 nM and 10 μM in the serum of a mammal.

Typically, dosages which may be administered in a method of the invention to a mammal, preferably a human, range in amount from 0.01 g to about 50 mg per kilogram of body weight of the mammal, while the precise dosage administered will vary depending upon any number of factors, including but not limited to, the type of mammal and type of disease state being treated, the age of the mammal and the route of administration and the specific agent or agents that are utilized. Preferably, the dosage of the compound will vary from about 0.1 g to about 10 mg per kilogram of body weight of the mammal. More preferably, the dosage will vary from about 1 g to about 5 mg per kilogram of body weight of the mammal. For example, in some embodiments, the dosage will vary from about 0.005 mg to about 5 mg per kilogram of body weight of the mammal.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The pharmaceutical composition can contain from about 0.01 to about 99 percent of the potassium channel blocker (e.g., 4-amino pyridine or a derivative or analog thereof), together with the carriers and/or excipients. For example, the amount of potassium channel blocker (e.g., 4-aminopyridine, a derivative thereof, or a combination thereof), by weight of the pharmaceutical composition can be about 0.1% or greater, about 1% or greater, about 2% or greater, about 5% or greater, about 10% or greater, about 15% or greater, about 20% or greater, about 25% or greater, about 30% or greater, about 50% or greater, about 75% or greater, or about 90% or greater.

In some embodiments, the pharmaceutical composition is defined by its ability to achieve serum therapeutically effective concentrations of 4-aminopyridine or a derivative thereof. In some embodiments, such concentrations range from about 10 nM to about 1 M 4-aminopyridine, or higher concentrations if combined with an anti-convulsant. Desired serum concentrations of 4-aminopyridine derivatives are defined by the ability of such agents to cause the desired therapeutic benefits without causing unacceptable side effects.

The pharmaceutical compositions described herein are used in a “therapeutically effective amount” of the potassium channel blocker (e.g., 4-aminopyridine, a derivative thereof, or a combination thereof). In some embodiments, the pharmaceutical composition can be formulated, such that when administered, it delivers a therapeutically effective amount of the potassium channel blocker (e.g., 4-aminopyridine, a derivative thereof, or a combination thereof) in an amount of 2.5 mg or greater. For example, the pharmaceutical composition when administered can deliver 3 mg or greater, 4 mg or greater, 5 mg or greater, 6 mg or greater, 7 mg or greater, 7.5 mg or greater, 8 mg or greater, 9 mg or greater, 10 mg or greater, 15 mg or greater, 20 mg or greater, 25 mg or greater, 30 mg or greater, 35 mg or greater, 40 mg or greater, 45 mg or greater, 50 mg or greater, 55 mg or greater, 60 mg or greater, 65 mg or greater, 70 mg or greater, 75 mg or greater, 80 mg or greater, 85 mg or greater, 90 mg or greater, or 95 mg or greater of potassium channel blocker (e.g., 4-aminopyridine, a derivative thereof, or a combination thereof).

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, and whether the administration is used to treat the dysfunction or to identify individuals for whom a full course of treatment is beneficial, etc.

In another aspect of the invention, the methods disclosed herein can be used to identify individuals who will benefit from a treatment with potassium channel blockers (e.g., 4-AP, a derivative of 4-AP, or any combination thereof). In some embodiments, individuals manifesting dysfunction in one or more tissues following an exposure to toxic insults of human-generated origin may be treated with the methods disclosed herein for between one and ten days to determine if treatment provides improvement in tissue function. Thus, in some embodiments, the methods disclosed herein can be used to provide personalized targeting of therapies.

When the therapeutic agents of the invention are prepared for administration, they are preferably combined with a pharmaceutically acceptable carrier, diluent, or excipient to form a pharmaceutical formulation, or unit dosage form. The total active ingredients in such formulations include from 0.1 to 99.9% by weight of the formulation. A “pharmaceutically acceptable” carrier, diluent, or excipient is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

It will be appreciated that the unit content of active ingredient or ingredients contained in an individual aerosol dose of each dosage form need not in itself constitute an effective amount for treating the particular indication or disease since the necessary effective amount can be reached by administration of multiple dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The compounds of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

The present invention also provides pharmaceutical compositions comprising one or more of the compositions described herein. Formulations may be employed in admixtures with conventional excipients, i.e., pharmaceutically acceptable organic or inorganic carrier substances suitable for administration to subject. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined when desired with other active agents, e.g., analgesic agents or anti-convulsants.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from benzyl alcohol, sorbic acid, parabens, imidurea, or any combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In exemplary embodiments, a pharmaceutical composition comprises a pharmaceutically acceptable excipient, such as a pharmaceutically acceptable carrier, and an exemplary compound described herein.

In certain exemplary embodiments, the pharmaceutical composition comprises, or is in the form of, a pharmaceutically acceptable salt, as generally described below.

The exemplary compounds can be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a subject, cleaves to form a free hydroxyl group.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Controlled- or sustained-release formulations of a pharmaceutical composition of the invention may be made using conventional technology.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

Transdermal formulations can also be prepared in the form of creams, ointments, salves, sprays, gels, lotions, emulsions, and transdermal patches. Such compositions may contain one or more chemical penetration enhancers, membrane permeability agents, membrane transport agents, emollients, surfactants, stabilizers, and combination thereof.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Liquid pharmaceutically administrable compositions can, for example, be prepared by dissolving, dispersing, etc., a composition as described herein and optional pharmaceutical adjuvants in an excipient, such as, for example, water, saline aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, referenced above.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more.

In another embodiment, the compositions of the invention are administered to the subject in range of dosages that include, but are not limited to, once every day, every two, days, every three days to once a week, and once every two weeks. In another embodiment, the compositions of the invention are administered to the subject in range of dosages applied in sustained release formulations that include, but are not limited to, once every two days, every three days to once a week, and once every two weeks, depending on the precise sustained formulation used and with the goal of maintaining serum concentrations of the therapeutic agents that are therapeutically effective without inducing unacceptable side effects.

It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

In some examples, the composition can be in the form of beads, films, or some other shape, as would be understood by a person of ordinary skill in the art. The size of each individual bead, film, or other shape is of a suitable size for implantation or other form of administration, as would be understood by a person of ordinary skill in the art. Further, the size of each individual bead, film, or other shape may be substantially consistent, or there may be a distribution of different sizes of the respective shape. Optionally, the beads can be implanted, ingested, or otherwise placed inside the body in some way, such that the agent is administered locally or systemically in a sustained-release manner.

It will be appreciated that the composition of the invention may be administered to a subject either alone, or in conjunction with another agent.

In certain embodiments, administration of a composition of the present invention may be performed by single administration or boosted by multiple administrations.

In one embodiment, the invention includes a method comprising administering a combination of compounds described herein. In certain embodiments, the combination has an additive effect, wherein the overall effect of the administering the combination is approximately equal to the sum of the effects of administering each compound. In other embodiments, the combination has a synergistic effect, wherein the overall effect of administering the combination is greater than the sum of the effects of administering each compound.

Optionally, the composition may comprise formulation suitable for delivering the treatment in sustained release formulation capable of releasing the therapeutic substance over period of time period lasting from several hours to several weeks. Such sustained release formulations may consist of osmotic pumps, a fibrin glue, a biocompatible polymer or hydrogel or other means of delivering treatment in a formulation that enables sustained release. The compound can be encapsulated in the polymer or hydrogel such that the agent is slowly released in the body to at least one portion of the tissue or tissues damaged by the toxic insult. Optionally, the compound can be dispersed throughout the polymer or hydrogel in such a manner to result in slow, sustained release as the polymer or hydrogel degrades inside the body. In some examples, the composition can comprise a biodegradable biocompatible polymer such as polyglycolide or polyglycolic acid (PGA), polylactide or polylactic acid (PLA), poly-L-lactic acid (PLLA), poly-D/L-lactic acid with polyglycolic acid (PDLLA-co-PGA), poly-L-lactic acid-co-glycolic acid (PLGA), PDLLA with bioactive glass, PLGA with bioactive glass, poly-L-lactic acid with (3-tricalcium phosphate (PLLA-TCP), poly-L-lactic acid with hydroxyapatite (PLLAHA), polydioxanone (PDS), polyethylene glycol (PEG), poly(8-caprolactone) (PCL), polycaprolactone (PCL) with alginate, polyhydroxybutyrate (PHB), polycarbonate (PC), N-vinyl pyrrolidone copolymers, polyorthoester, chitosan, poly(2-hydroxyethyl-methacrylate) (PHEMA), hyaluronic acid and hydrogels.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: 4-Aminopyridine for the Treatment of Tissue Dysfunction Caused by Toxic Insults

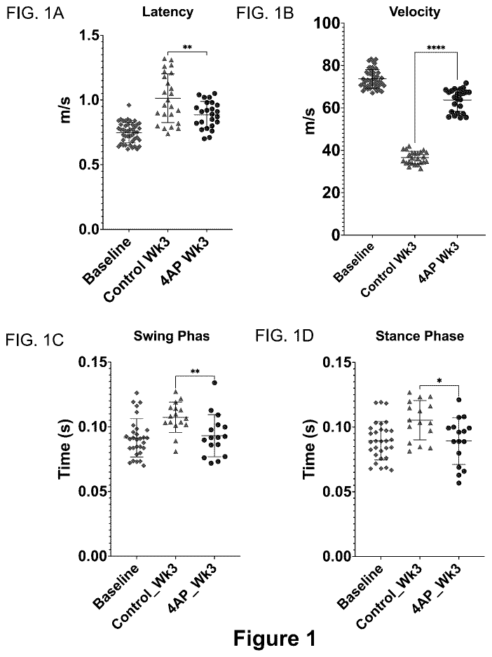

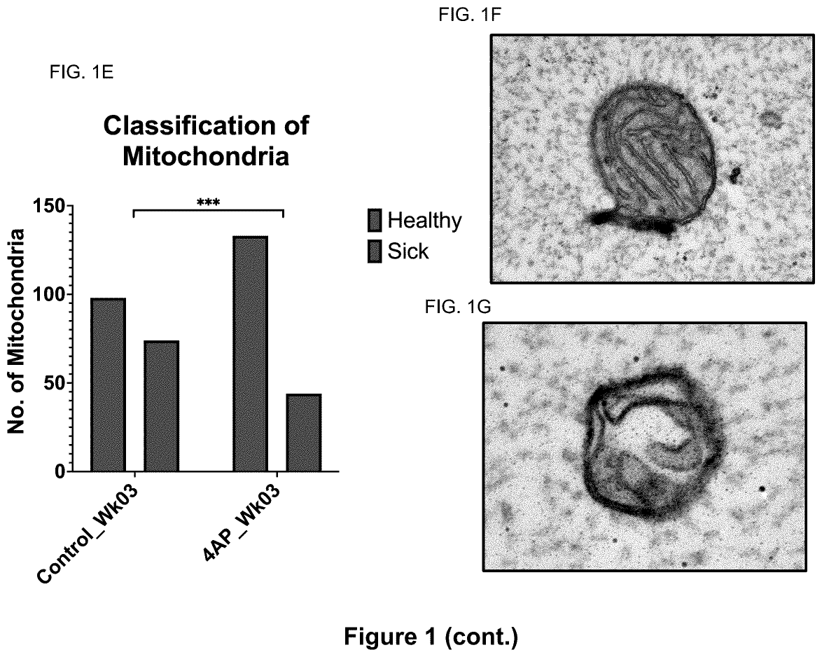

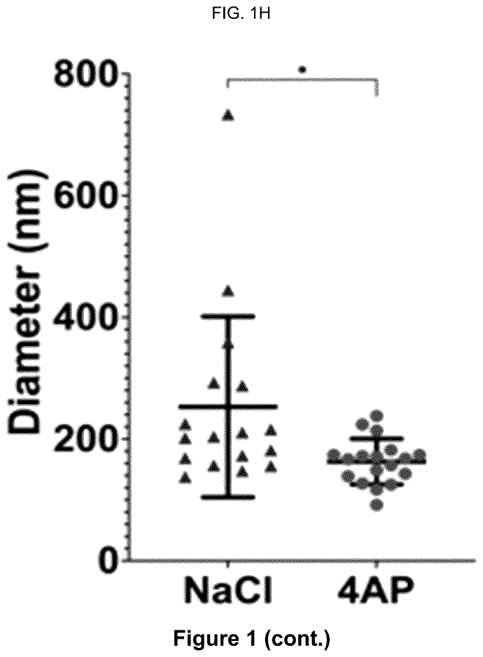

The present examples demonstrate studies in vivo of a nature relevant to multiple toxic insults. More specifically, initial studies were conducted on the specific case of CIPN as induced by exposure to PTX. FIG. 1, comprising FIG. 1A through FIG. 1H, depicts representative results demonstrating that 4-aminopyridine (4-AP) conferred protection against developing a chemotherapy-induced peripheral neuropathy (CIPN) and mitochondrial damage caused by clinically relevant doses of PTX. 16 week old C73L/6 female mice were given a single dose of 35 mg/kg PTX (i.p.). Animals were separated into control (saline, i.p., n=4) or treatment group (2 mg/kg 4-AP daily, i.p., n=4). Pre-treatment Catwalk scores were compared to 21 days post-PTX injection.

FIG. 1A depicts representative nerve conduction studies that showed significant increases in latency in the control group over the study. In 4-AP treated mice, there was a significantly smaller increase in latency. FIG. 1B depicts representative nerve conduction studies that showed significant decreases in velocity in the control group over the study. In 4-AP treated mice, there was a significantly smaller decrease in velocity. FIG. 1C depicts representative catwalk analysis that demonstrated significant increases in swing phase in control mice, but no significant changes in the 4-AP treated group (p<0.05; p<0.01; p<0.001). FIG. 1) depicts representative Catwalk analysis that demonstrated significant increases in stance phase in control mice (indicative of gait abnormalities), but no significant changes in the 4-AP treated group (*p<0.05; **p<0.01; ***p<0,001).

To align with clinical treatments, mice were treated with a PTX dose of 35 mg/kg (equivalent to approximately 110 mg/m² and within the clinical treatment range) for four cycles, each three weeks apart. The results from this preliminary study (n=4) demonstrated that the animals tolerated the dose, and the concentration was sufficient to induce clinically significant signs of CIPN (FIG. 1). A different cohort of mice was next treated with 35 mg/kg PTX and continued for one cycle (three weeks), which was also found to cause CIPN.

The studies also included a 4-AP treatment arm (n=4) with a daily dose of 2 mg/kg 4-AP administered i.p. (as per most previous studies by MN and colleagues). It was found that the 4-AP group exhibited significantly reduced signs of CIPN compared with control mice given an equivalent volume injection of saline i.p. The studies used a low dose of 4-AP that corresponds with −40% of the mouse body surface area equivalent of the dosage of 20 mg/day used in treating multiple sclerosis and is smaller than equivalent doses examined in patients with chronic spinal cord injuries.

In these studies, it was found that 4-AP treatment improved CIPN-related changes in motor function (by Catwalk analysis) and electrophysiological parameters caused by PTX treatment, and decreased PTX-induced changes in mitochondrial size and health. To evaluate detailed functional changes in gait, the CatWalk™ (Noldus Information Technology, Wageningen, The Netherlands) method was implemented. Electrophysiological data relevant to nerve conduction studies also were recorded, and both latency and velocity were improved in the animals treated with 4-AP.

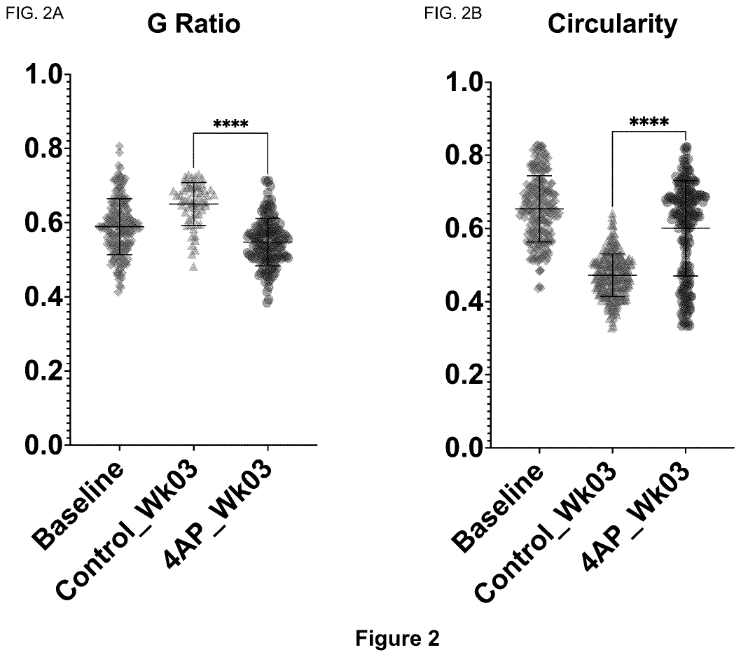

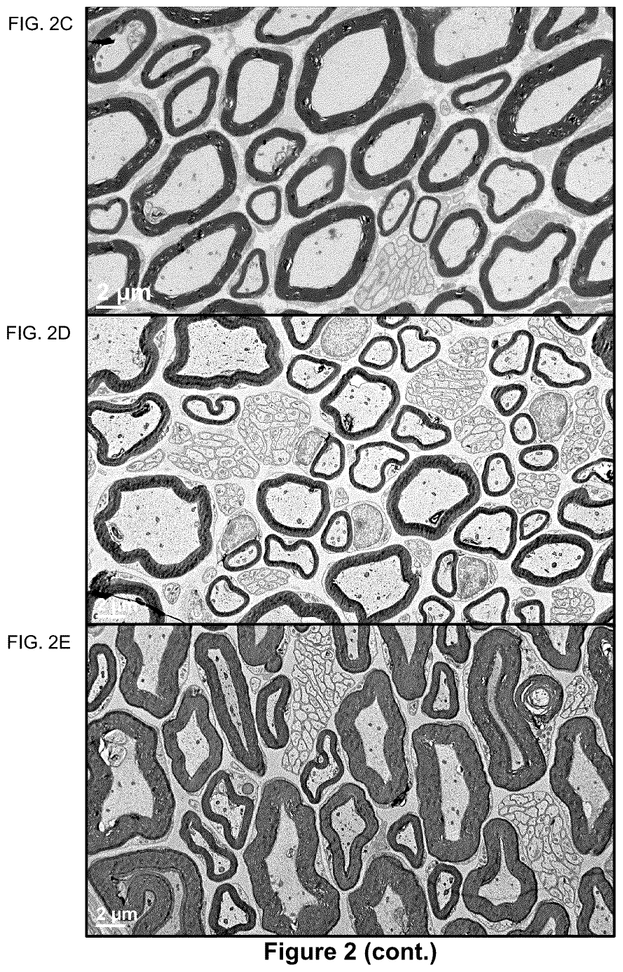

It is also demonstrated herein that concurrent 4-AP treatment prevents PTX-induced changes in peripheral nerve function associated with CIPN. Sciatic nerve samples were harvested and resin-embedded, sectioned and imaged using transmission electron microscopy. Images were analyzed to calculate markers of peripheral neuropathy, namely: axon counts, fiber size, myelination (g ratio), and circumferential irregularity (FIG. 2). Mitochondria were evaluated to assess chemotherapy induced damage and the results demonstrated that 4-AP improved myelination. FIG. 2, comprising FIG. 2A through FIG. 2E, depicts representative results demonstrating that 4-AP prevented chemotherapy induced axonal damage. FIG. 2A depicts representative G ratio (axon:myelin area) data demonstrating that axons within the 4-AP treatment were better myelinated than the control group. FIG. 2B depicts representative circularity data demonstrating that axons within the 4-AP treatment also were more regularly structured than the control group. FIG. 2C depicts representative baseline appearance of sciatic nerve. FIG. 2D depicts representative appearance of control axons 3-weeks following one treatment of 35 mg/kg PTX. FIG. 2E depicts representative appearance of 4-AP (2 mg/kg daily) treated axons demonstrating thicker myelin and more regular structure.

### Example 2

A second study using a longer time course of administration of PTX further demonstrates the ability of 4-AP to prevent the occurrence of CIPN when administered concurrently.

All animals received four injections of 35 mg/kg i.p. of PTX (equivalent to approximately 110 mg/m² and within the clinical treatment range, and delivered in the form of a dilution of clinically used Taxol), at three-week intervals. A subset of animals concurrently received 4-AP (1 mg/kg, daily), which was started at the same as the initial PTX treatment and continued throughout. Functional outcome responses were recorded at three-week intervals throughout the study period, and included von Frey monofilament testing, cold plate thermal sensitivity, CatWalk™ gait analysis, and nerve conduction testing. All animals had functionally demonstrable peripheral neuropathy at six weeks.

It was observed in these experiments that concurrent treatment with 4-AP prevented development of PTX-induced CIPN. 4-AP treatment prevented PTX-induced changes in multiple motor functions and electrophysiological parameters. Analysis of response to thermal changes, using a cold-plate analysis, showed that 4-AP prevented development of CIPN-like changes regardless of whether the changes indicated hyperalgesia or hypoalgesia. Analysis of mechanical allodynia with Von Frey filament testing revealed an increased sensitivity, as indicated by response to less force. This change was apparent by three weeks, worsened at six weeks, and plateaued at for the next six weeks. In contrast, mice treated with PTX+4-AP showed no changes from baseline in their response, with the differences from the PTX alone group being statistically significant at all time points. A significantly increased sensitivity to cold, as determined by the number of times mice tried to jump off of the cold plate stimulus, was apparent at six weeks after initiation of treatment and was maintained throughout the remaining six weeks of analysis. In contrast, mice treated with PTX+4-AP showed no changes from baseline in this parameter. The differences between the two treatment groups was highly statistically significant at Weeks 6, 9 and 12. Other responses to cold indicated a loss of sensitivity in PTX-treated mice, as determined by number of times mice would lift their paws off of the cold plate, or would lick their hind paws. For these outcomes also, concurrent treatment with 4-AP prevented the development of these changes, with differences between PTX vs. PTX+4-AP groups being statistically significant.

Treatment with CIPN in these experiments also caused decreases in the amplitude and velocity of nerve impulse conduction, with large changes apparent at 6 weeks after the initiation of PTX treatment and continued for the next six weeks. For both of these parameters also, concurrent treatment with 4-AP prevented the development of these changes, with differences between PTX vs. PTX+4-AP groups being statistically significant.

### Example 3: 4-AP Treatment Prevents PTX-Induced CIPN Caused by Repetitive PTX Exposure, Even when Applied at Very Low Levels of 4-AP

It is critical to know whether 4AP treatment can also prevent PTX-induced peripheral neuropathy when repetitive treatment with PTX occurs. This is the situation that would occur in the clinic, where cancer patients are not treated with a single dose of paclitaxel but instead undergo repetitive exposures. There is no prior indication of whether 4AP might be useful in preventing the effects of this unusual type of injury which has elements of a repetitive injury due to the repetitive exposure to paclitaxel and also elements of a chronic injury because of the accumulation of damage that occurs. Thus, in this situation, 4-AP needs to overcome existing damage and prevent further damage in order to ameliorate the chemotherapy-induced changes.

To determine whether 4-AP also conferred protection against developing CIPN when animals were repetitively exposed to PTX, 16 week old C7BL/6 female mice were given four cycles of PTX treatment at doses of 35 mg/kg PTX (i.p.). Animals were separated into control (saline, i.p.) or treatment group treated with a dose of 4AP 25% of that used in Examples 1 and 2 (i.e., 0.5 mg/kg 4-AP daily, i.p.). Pre-treatment Catwalk scores were compared to 21 days post-PTX injection.

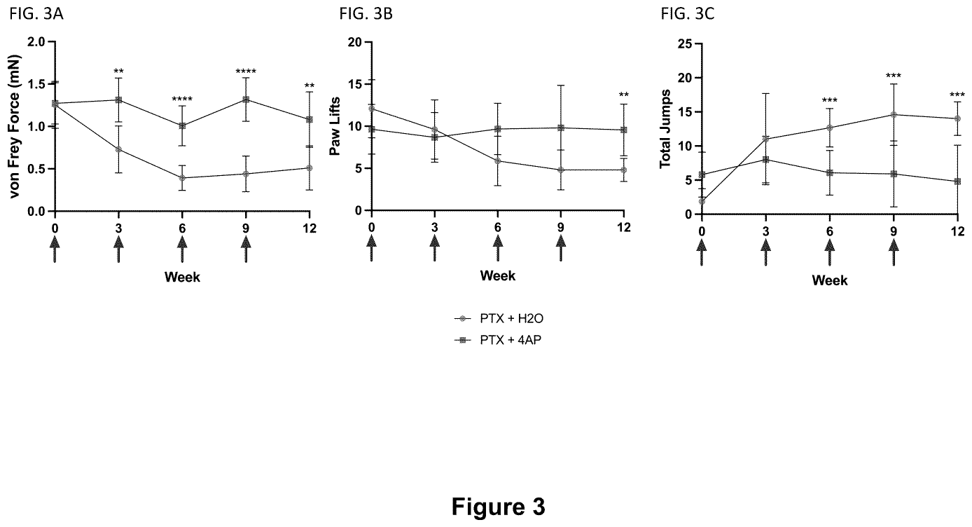

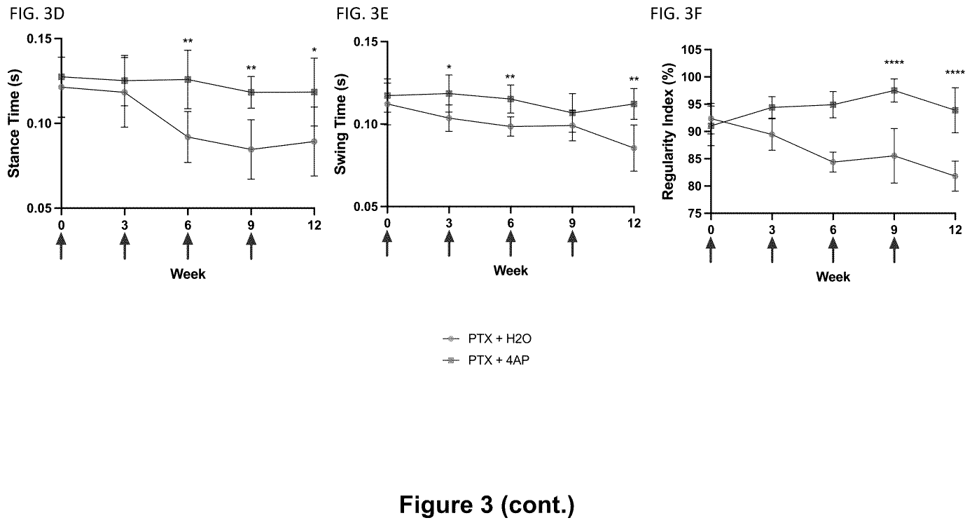

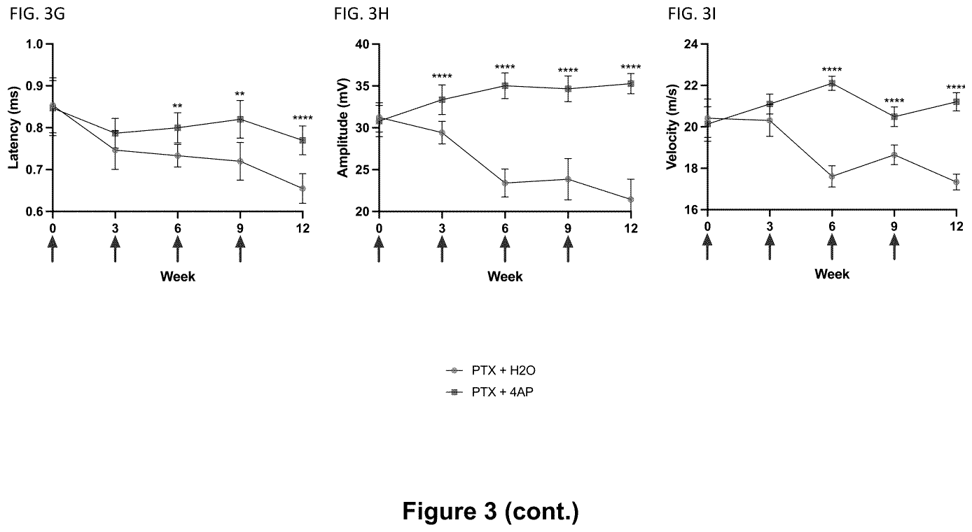

Analyses revealed that 4-AP treatment ameliorated PTX-induced mechanical hypersensitivity as tested by application of von Frey filament testing, where the sensitivity to smaller filaments reveals increased sensitivity to painful stimuli. Outcomes were highly significant at Weeks 3, 6, 9 and 12. 4-AP treatment also prevented development of the opposite symptoms of hyposensitivity to stimuli, using analysis of pawlifts and jumping behavior in response to cold-plate stimulus. Pawlift outcomes trended significant at Weeks 6 and 9 and were highly significant at Week 12. Jumping behavior was highly significant at Weeks 6, 9 and 12 (FIG. 3A-FIG. 3C).

4-AP treatment also ameliorated PTX-induced gait abnormalities detected by Catwalk analyses, with significant effects at multiple time points (FIG. 3D-FIG. 3F). 4-AP treatment also ameliorated PTX-induced abnormalities in nerve conduction in the multiple outcomes of latency (FIG. 3G), amplitude (FIG. 3H) and velocity (FIG. 3I).

### Example 4: 4-AP Treatment Prevents PTX-Induced Peripheral Nerve Structural Changes Caused by Repetitive PTX Exposure, Even when Applied at Very Low Levels of 4-AP

It is critical to know whether 4AP treatment can also prevent PTX-induced peripheral neuropathy when repetitive treatment with PTX occurs. This is the situation that would occur in the clinic, where cancer patients are not treated with a single dose of paclitaxel but instead undergo repetitive exposures. There is no prior indication of whether 4AP might be useful in preventing the effects of this unusual type of injury which has elements of a repetitive injury due to the repetitive exposure to paclitaxel and also elements of a chronic injury because of the accumulation of damage that occurs.

It is also particularly important to determine whether or not such treatment prevents the structural damage that is associated with the development of CIPN. Nothing is known about the ability of 4-AP treatment to prevent damage caused by cancer drugs, which have many different kinds of effects on cells. It's known that a great deal of structural damage occurs in association with chemotherapy treatment, but it is impossible to predict from current information whether or not 4AP would have any benefits in preventing such types of damage.

To determine whether 4-AP prevented chemotherapy-induced structural damage even when chemotherapy was applied multiple times, ultrastructural analyses of tissue from mice treated with 35 mg/kg PTX, every 3 weeks, for 4 cycles of treatment was conducted. A low dose of 4-AP that was only 25% of the dose used in Examples 1 and 2 was used, which was applied at 0.5 mg/kg daily instead of 2 mg/kg daily.

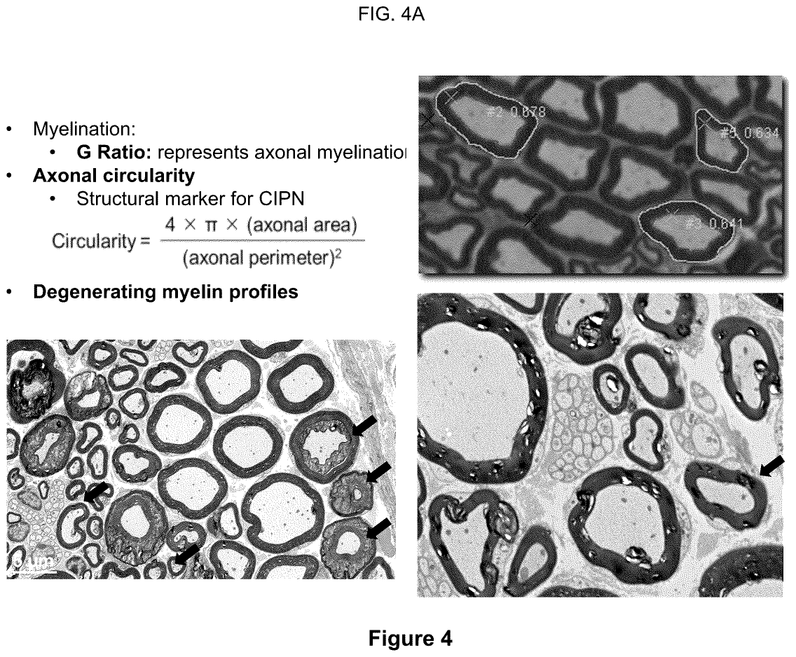

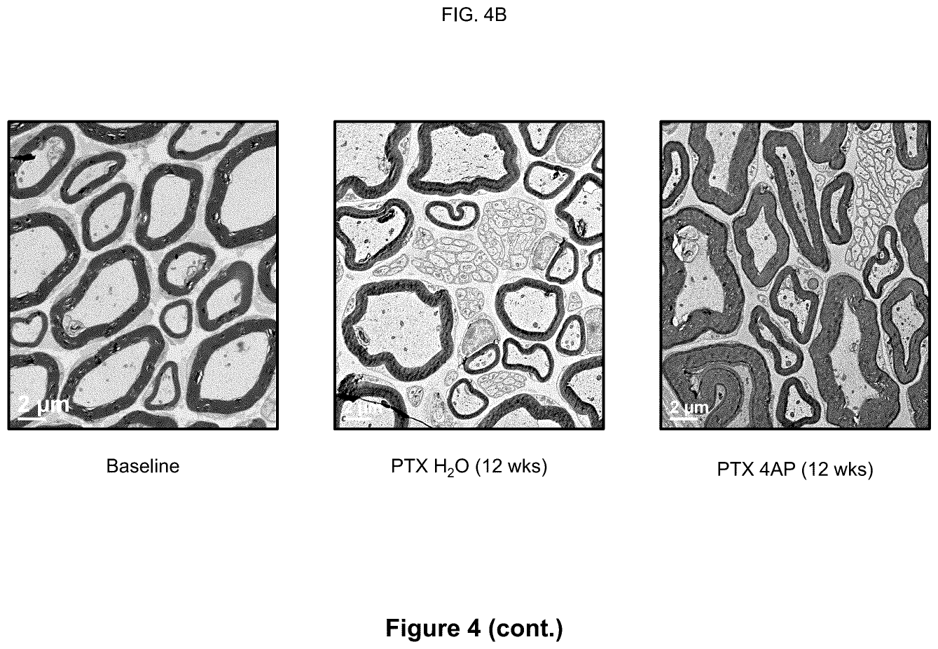

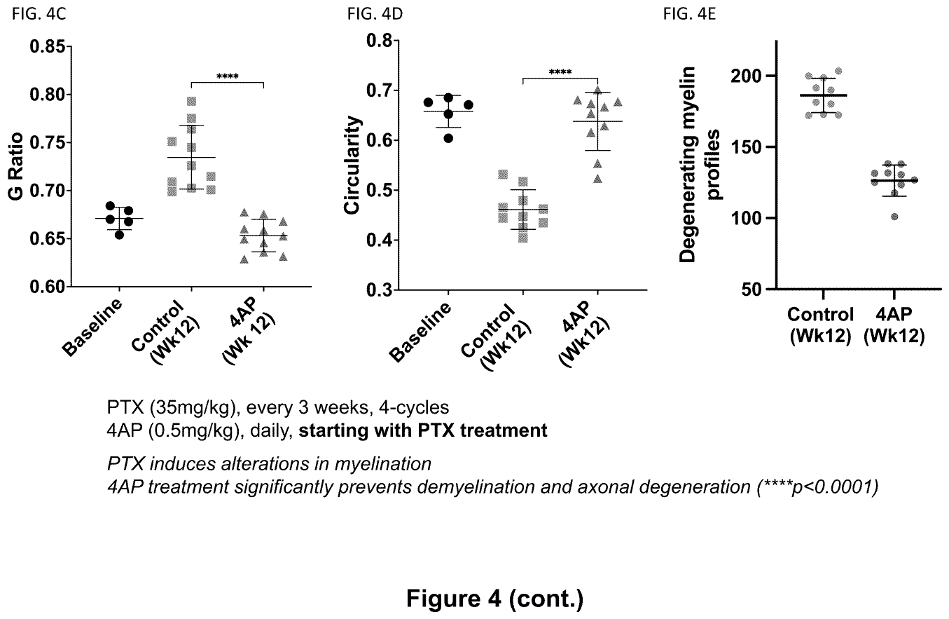

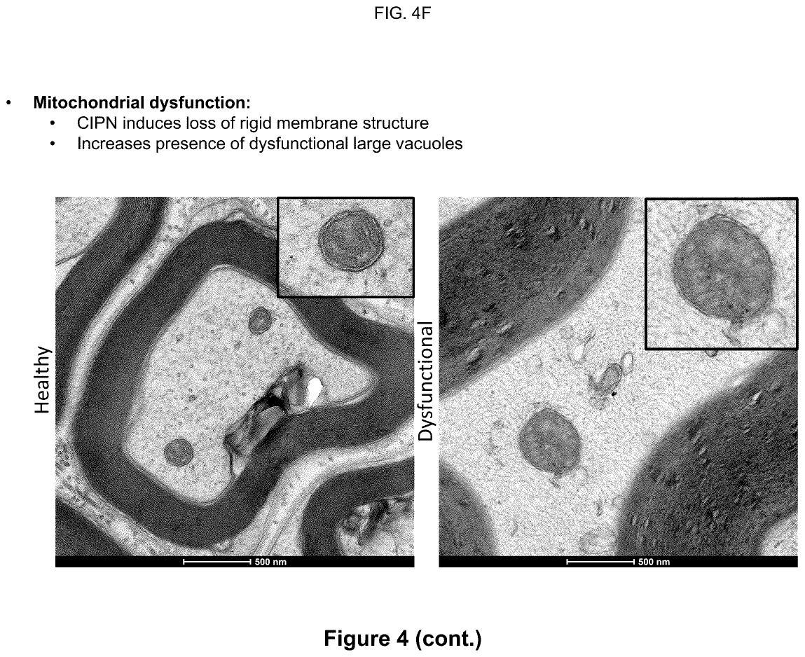

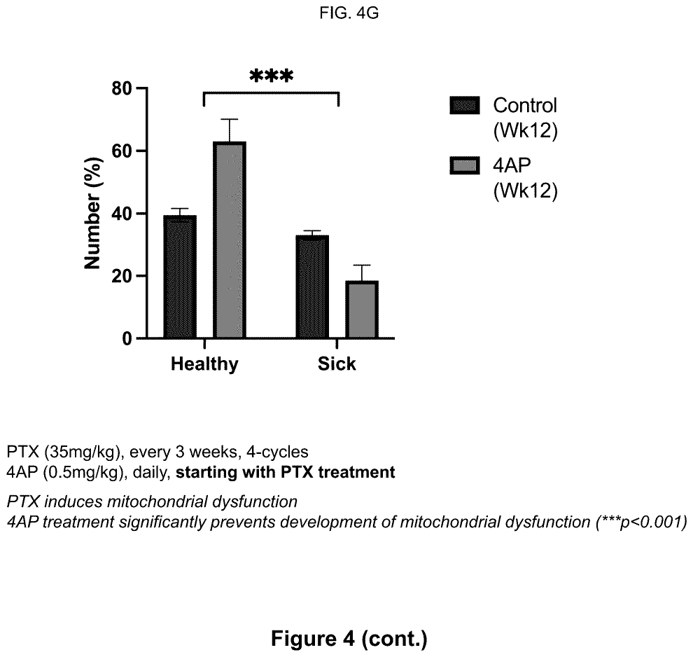

Ultrastructural analyses of myelination conducted revealed multiple benefits of 4-AP treatment (FIG. 4). Analysis of the G ratio (axon:myelin area) data demonstrated that axons within the 4-AP treated mice were better myelinated than the control (PTX+Saline) group (FIG. 4C). Analyses of circularity revealed that axons within the 4-AP treatment also were more regularly structured than the control group (FIG. 4D). The 4-AP treated group also had a decreased frequency of degenerating myelin profiles.

These experiments also revealed that treatment with 4AP improves mitochondrial health as determined by mitochondrial structure, even in the situation of repetitive exposure to chemotherapy.

### Example 5: 4-AP Treatment Reverses PTX-Induced CIPN Even after the Peripheral Neuropathy has been Established, and is Effective at Both Functional and Histological Levels of Analysis

Another important question to consider is whether delayed treatment with 4AP can overcome CIPN after it has been established. This would be a situation relevant to the treatment of ongoing chemotherapy, which is highly clinically relevant because peripheral neuropathy can be dose limiting in cancer treatment. When it occurs, the patient may defer necessary treatment because of the effects of CIPN on their quality of life.

There is no prior indication as to what 4AP might be capable of doing in this situation because the treatment with therapy constitutes a repetitive injury, in which damage begins with the first treatment and continues to increase with subsequent treatments. Prior studies on the use of 4AP in treating established syndromes, such as multiple sclerosis and spinal cord injury, have demonstrated limited results in this regard. As discussed elsewhere, in particular the effects of 4AP on mitigating pain-related symptoms seem likely to be limited at best and more likely to be ineffective.

To examine this question, 16 week old C7BL/6 female mice were given four cycles of PTX treatment at doses of 35 mg/kg PTX (i.p.). Animals were separated into control (saline, i.p.) or treatment group treated with a dose of 4AP 25% of that used in FIGS. 1 and 2 (i.e., 0.5 mg/kg 4-AP daily, i.p., with treatment initiated 6 weeks after the first PTX treatment and when CIPN-related changes were already apparent).

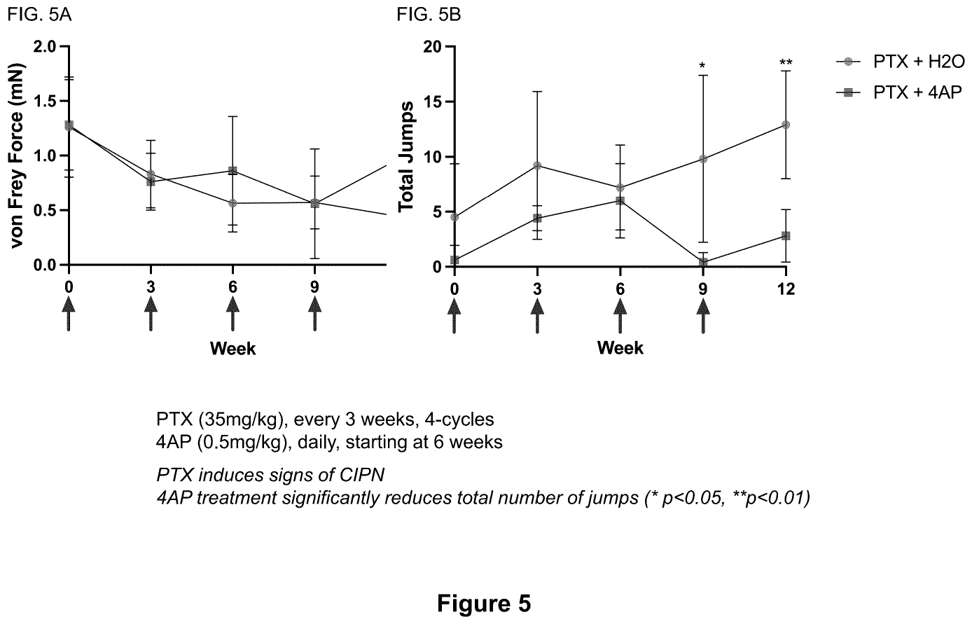

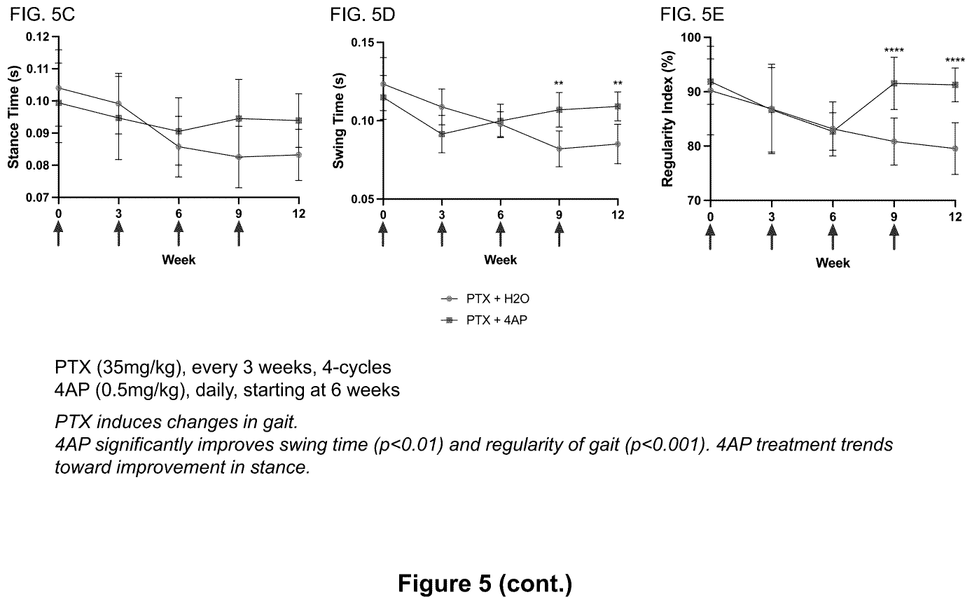

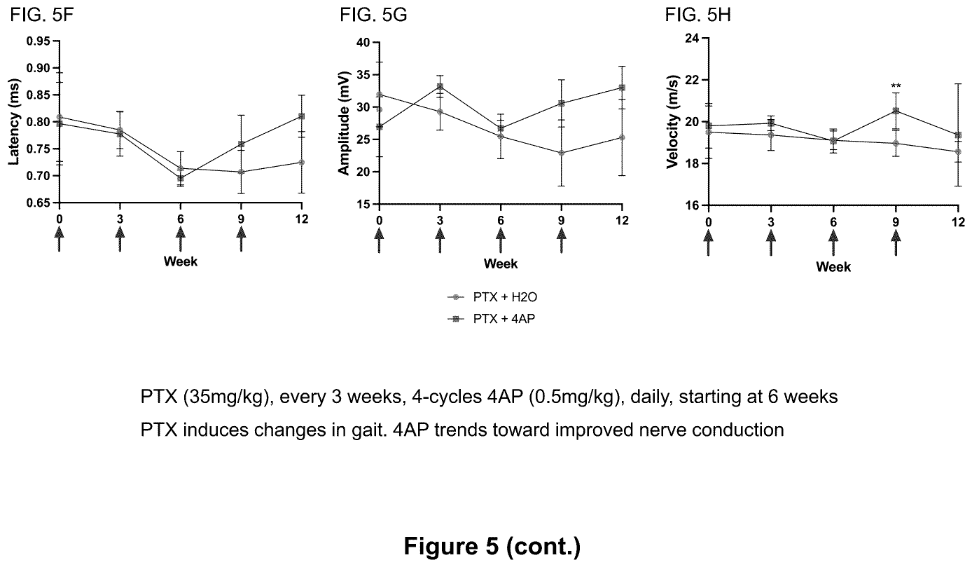

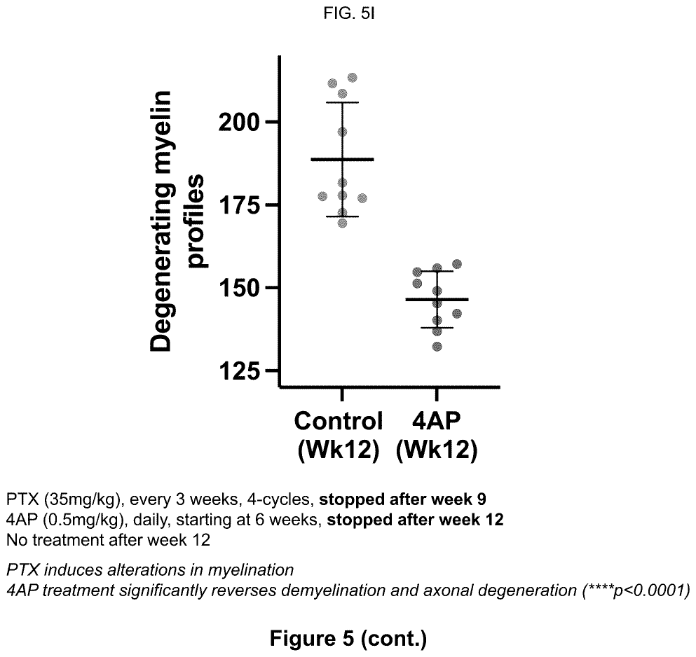

These experiments revealed that 4-AP treatment also reverses the effects of repetitive PTX treatment on peripheral nerve, with 4-AP treatment starting at 6 weeks when mice received their third exposure to PTX. For example, 4-AP treatment on PTX-induced mechanical hypersensitivity as tested by application of von Frey filament testing (where the sensitivity to smaller filaments reveals increased sensitivity to painful stimuli) revealed that benefits of 4-AP treatment began to be observed at Week 12 (FIG. 5A). 4-AP treatment also reversed the symptoms of hypersensitivity to thermal stimuli, using analysis of jumping behavior (FIG. 5B) in response to cold-plate stimulus. Changes in jumping behavior were significant at Weeks 9 and 12.

Treatment with 4-AP also reversed PTX-induced gait abnormalities detected by Catwalk analyses, with significant effects at weeks 9 (the first analysis after 4-AP treatment started) and 12 for swing time and regularity index, and outcomes for stance time trending towards significance at these same time points (FIG. 5C-FIG. 5E).

Treatment with 4-AP also reversed PTX-induced abnormalities in nerve conduction after they are established by multiple rounds of PTX exposure in the multiple outcomes of latency (FIG. 5F), amplitude (FIG. 5G) and velocity (FIG. 5H).

Treatment with 4-AP also reversed PTX-induced structural myelin abnormalities indicative of myelin degeneration (FIG. 5I). This result reveals the unexpected ability to promote tissue repair after CIPN has already been established.

### Example 6: 4-AP Treatment Durably Reverses PTX-Induced CIPN Even after the Peripheral Neuropathy has been Established, and Benefits Remain after Treatment is Stopped

One of the important problems in treating damage induced by exposure to toxic agents is that treatments are needed that are capable of reversing damage that has already occurred. Many people are not diagnosed with damage until after the injury has occurred. Moreover, in the specific case of peripheral neuropathy induced by exposure to chemotherapeutic agents, the neuropathy can manifest after treatment has stopped. In addition, it may be desirable to delay initiation of treatment for the neuropathy until the cancer treatment has been completed.

In respect to treatment of damage with 4-AP, there is no prior information that allows prediction of outcomes of established damage. In fact, the predictions based on years of study of chronic injuries would predict no such benefits. This is because in such syndromes as multiple sclerosis, chronic spinal cord injuries, spinocerebellar ataxias and other established conditions where 4AP has been examined to provide symptomatic relief, the benefits of 4AP treatment disappear when treatment stops.

Thus, the question of whether or not treatment of CIPN with 4AP can promote recovery that is retained after treatment is completed is not predictable. This lack of predictability is exacerbated by the fact that chemotherapy treatment is itself a repetitive injury and has an established chronic component and is also exacerbated with each further treatment. This is a very different situation than in prior analyses of the effects of 4-AP treatment. Nonetheless, prior studies on established damage would predict that when treatment with 4-AP is initiated weeks or longer after the initial damage occurs that durable recovery would not occur. Thus, on top of the lack of data indicating whether 4-AP could provide benefits of any sort in the treatment of CIPN, there is no ability to predict whether any benefits that did occur would be durable after treatment stopped, a situation that would require tissue repair to occur in the context of treating an established injury.

To examine this question, 16 week old C7BL/6 female mice were given four cycles of PTX treatment at doses of 35 mg/kg PTX (i.p.). All animals had functionally demonstrable peripheral neuropathy at six weeks. Animals were then treated with the same dose and treatment regimen of 4-AP used in FIG. 5. Treatment with 4-AP ended at Week 12 after the first exposure to PTX and after 6 weeks of treatment with 4-AP, and animals were observed for an additional six weeks after this point (i.e., for 6 weeks with no 4-AP treatment).

These experiments revealed the unexpected result that 4-AP treatment can reverse effects of repetitive rounds of PTX exposure on peripheral nerve function in a manner that is retained even after treatment ends. Benefits were still present six weeks after the end of treatment, as contrasted with predicted elimination of >99.9% 4-AP over 24 hours (by renal clearance). Such durable changes are indicative of pro-reparative effects of 4-AP treatment that extend beyond providing symptomatic relief that only is present during the time of treatment.

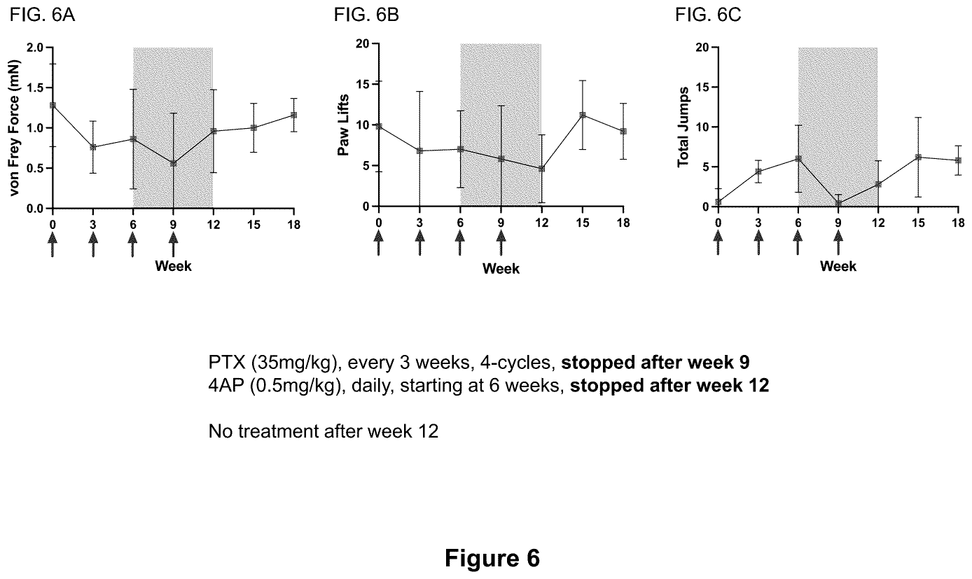

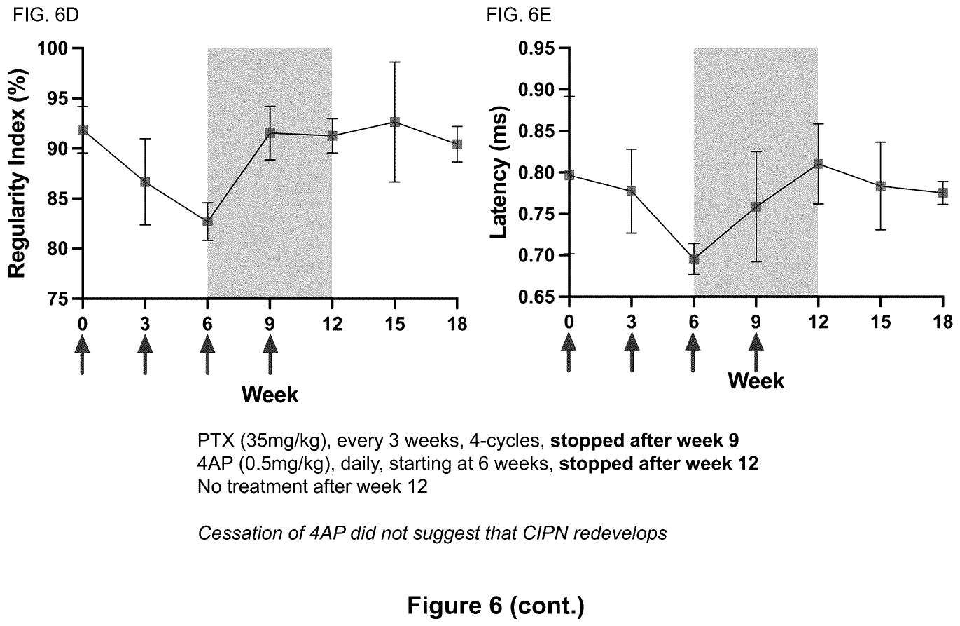

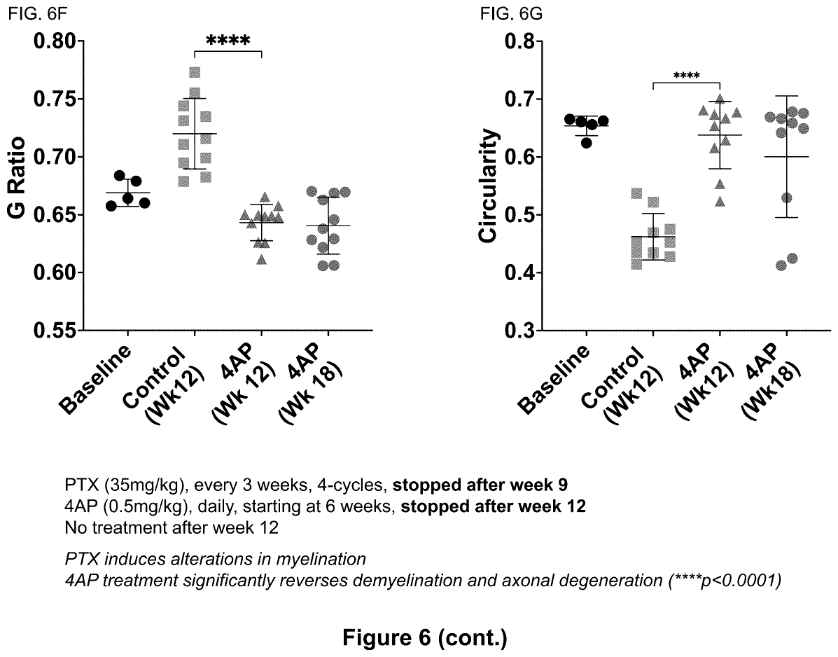

The results of these experiments demonstrated that 4-AP treatment restores normal function after several weeks of PTX treatment regardless of whether the symptoms are hyperalgesia or hypoalgesia, which is the most promising of all outcomes in respect to addressing clinical needs. FIG. 6A depicts the effects of 4-AP treatment on PTX-induced mechanical hypersensitivity as tested by application of von Frey filament testing, where the sensitivity to smaller filaments reveals increased sensitivity to painful stimuli. Benefits began to be observed at Week 12 and were maintained at Weeks 15 and 18. FIG. 6B shows that 4-AP treatment also causes durable reversal the opposite symptoms of hyposensitivity to stimuli, using analysis of paw lifts (FIG. 6B) and jumping behavior (FIG. 6C) in response to cold-plate stimulus.

FIG. 6D depicts the ability of 4-AP to durably reverse PTX-induced gait abnormalities as determined by the Catwalk Regularity Index, with benefits observed at Week 9 (i.e, after 3 weeks of 4-AP treatment) and retained for at least 6 weeks after 4-AP treatment ended. FIG. 6E depicts the ability of 4-AP to reverse PTX-induced abnormalities in nerve conduction latency after they are established by multiple rounds of PTX exposure.

FIG. 6F-FIG. 6G depict the ability of 4-AP to cause durable pro-reparative changes when used to treat established PTX-induced CIPN and tissue damage. The changes in G-ratio (FIG. 6F) and circularity (FIG. 6G) caused by repetitive PTX exposure are restored to normal at the 12 week time point, and these benefits are maintained at the 18 week time point (i.e, 6 weeks after treatment has ended).

### Example 7: 4-AP Treatment Reverses Cisplatin-Induced CIPN

To determine whether the benefits provided by 4-AP treatment experiments were conducted on another toxic substance with a different mechanism of action than paclitaxel. In these experiments, the toxic agent studied was cisplatin. In contrast with the microtubule-stabilizing activity of the taxanes (which include paclitaxel), the platinum-containing compound cisplatin is thought to kill rapidly dividing cells by causing DNA cross-linking. Paclitaxel and cisplatin are also very different in their structures. The differences in the mechanisms of action of cisplatin and paclitaxel is underscored by the fact that paclitaxel is often used to treat cancers that are resistant to cisplatin. Thus, the present examples describe studies in vivo of a nature relevant to multiple toxic insults.

In these experiments, 16-week-old C7BL/6 female mice were treated with CIS (2.5 mg/kg, once weekly, i.p.) for eight weeks. Animals were then treated with 4-AP (1 mg/kg daily) for the next six weeks (n=4).

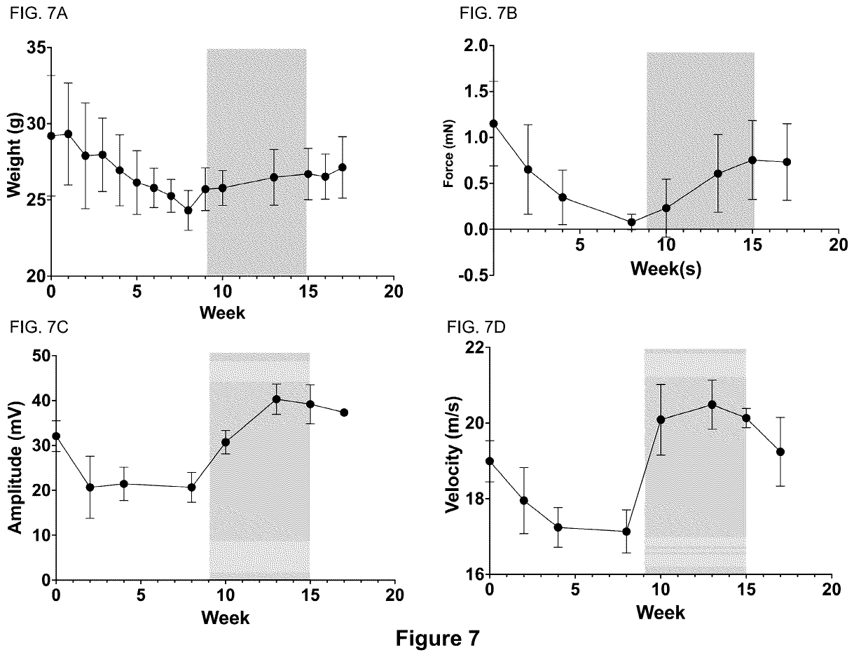

These experiments revealed that mice treated with 4-AP beginning 9 weeks after the initiation of CIS treatment regained normal body weight. CIS-treated mice express CIPN, and 4-AP is as effective at reversing CIS-induced CIPN as it was in reversing PTX-induced CIPN. Analysis of hyperalgesia by Von Frey filament analysis showed a dramatic increase in sensitivity in CIS treated mice, and a restoration of normal levels of sensitivity with 4-AP treatment. The restoration of normal sensitivity was durable and maintained for at least two weeks after treatment ended, well beyond the 12-16 hour time point when virtually all 4-AP would be expected to be cleared from the body, thus demonstrating durable recovery of function and indicating pro-reparative effects of the treatment. FIG. 7C depicts representative results demonstrating the effect of 4-AP on nerve impulse amplitude that was also restored by 4-AP treatment with improvements again retained for at least two weeks after treatment with 4-AP ended. FIG. 7D depicts representative results demonstrating the effect of 4-AP on nerve impulse velocity that was also restored by 4-AP treatment with improvements again retained for at least two weeks after treatment with 4-AP ended (as contrasted with predicted elimination of >99.9% 4-AP over 24 hours (by renal clearance)), thus demonstrating durable recovery of function and indicating pro-reparative effects of the treatment. This outcome can only be explained by 4-AP-induced reparative changes in damaged tissue.

### Example 8: 4-AP Treatment Prevents Chemotherapy-Induced Damage to the Kidney

Another tissue that is often damaged by treatment with chemotherapeutic agents is the kidney, and providing protection from such nephrotoxicity is of great importance. The combination of focus on the kidney and on the fact that exposure to such toxic insults as chemotherapeutic agents is a very different type of insult than occurs in traumatic injury and other previously studied uses of 4-AP makes it unpredictable as to whether benefits of 4-AP in the context of this invention would also include the kidney.

To examine effects on the kidney histological studies were conducted on this tissue. Mice received PTX on day 0 of the experiment, and half of those mice also were injected with 4-AP at this time, and every day thereafter, at a dosage of 2 mg/kg. Mice not receiving 4-AP were injected with saline to control for any stress induced by daily injections. Experimental groups also included mice receiving no PTX treatment, and treated only with daily injections of saline. Mice were sacrificed after 1 week. Mice were perfused with 4% paraformaldehyde and kidneys were sectioned and stained with haematoxylin & eosin (H&E).

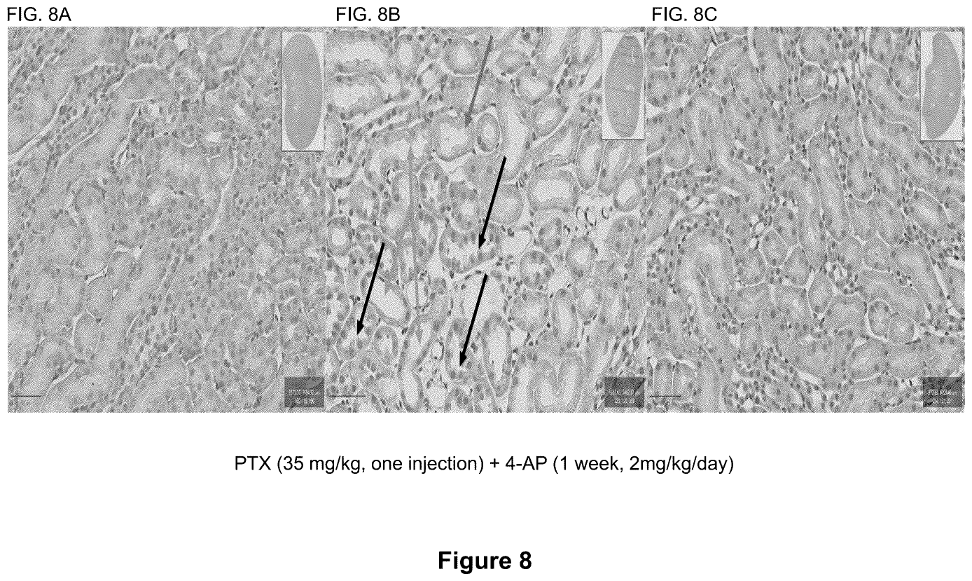

H&E staining of saline treated, PTX treated, and PTX+4-AP treated kidneys revealed normal proximal and distal tubules in both saline and PTX+4-AP treated kidneys (FIG. 8). Loss of brush borders (dark grey arrows), nuclear dropout (grey arrow), and blebbing of tubular epithelial cells (black arrows) were observed in PTX treated kidneys but not in kidneys isolated from animals treated with PTX+4-AP.

The results of this experiment demonstrated that 4-AP co-treatment also prevented multiple aspects of histological kidney damage caused by PTX exposure. This surprising result demonstrates that 4-AP-mediated protection against chemotherapy also is found in a tissue that is not part of the nervous system. These results further buttress the data provided in Table 1.

### Example 9: 4-AP Exposure Prevents PTX-Induced Changes in the Central Nervous System

Another tissue of importance in injury from anti-cancer agents, radiation and other forms of toxic insults is the central nervous system (CNS). The CNS is affected by many anti-cancer treatments, by radiation and by many toxic insults. Such damage can lead to changes in multiple neurological and/or cognitive functions, and preventing such damage is of great medical importance. Interventions to prevent such damage are lacking to an extent even greater than possible interventions for other parts of the body.

One of the first signs of damage to the CNS is increases in expression of glial fibrillary acidic protein (GFAP). Increases in GFAP are indicative of activation of astrocytes, and increase in response to inflammation, any of a variety of physical injuries and in response to chemical insults such as exposure to chemotherapeutic agents (e.g., Liu, et al., Paclitaxel-activated astrocytes produce mechanical allodynia in mice by releasing tumor necrosis factor-α and stromal-derived cell factor 1. J. Neuroinflammation 2019; 16: 209; Masocha, Astrocyte activation in the anterior cingulate cortex and altered glutarmatergic gene expression during paclitaxel-induced neuropathic pain in mice. PeerJ 2015 3:e1350).

To determine whether 4-AP treatment can prevent PTX-induced increases in a reactive inflammatory response in the brain, as detected by increased expression of glial fibrillary acidic protein (GFAP), 16-week-old female C57BL/6 mice were inoculated with a triple negative murine breast cancer cell line E0771. When tumors were palpable, the mice were injected with either paclitaxel and water, 4-AP, both, or saline as a control. Paclitaxel (35 mg/kg) was injected on day one and 4-AP and water were injected daily. The mice were sacrificed on day nine and their brains were perfused and harvested. Brains were cryosectioned coronally.

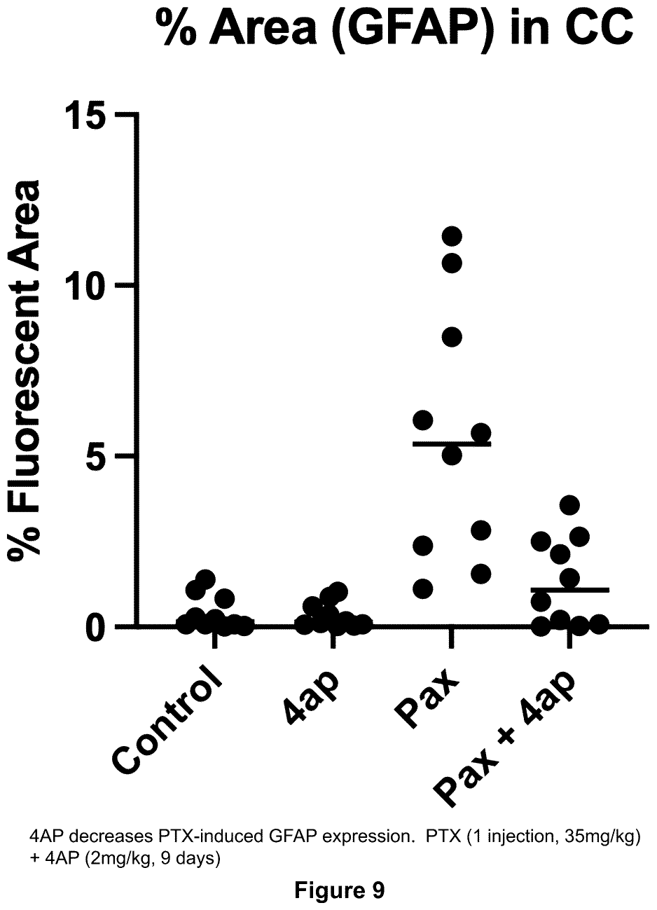

The mouse brain sections were stained with anti-GFAP antibodies, followed by a fluorescent secondary, for analysis via immunofluorescence. Images of the corpus callosum, the major myelinated tract in the CNS, were taken via a confocal microscopy and images were analyzed with Image-J. These experiments revealed that PTX treatment was associated with increased expression of GFAP (FIG. 9). In mice co-treated with 4-AP the increased expression of GFAP caused by PTX exposure was greatly reduced.

### Example 10: 4-AP's Protection Against Toxicity of Chemotherapeutic Agents is Selective for Normal Cells

One of the further unexpected properties of 4-AP is that its capacity to provide protection against chemotherapeutic agents with different mechanisms of action is selective for normal tissue (i.e., non-transformed cells), and 4-AP does not provide protection for cancer cells. This outcome was also unpredictable as many survival mechanisms are identical in normal cells and cancer cells. This surprising outcome is of critical importance because protecting cancer cells would greatly decrease the value of 4-AP as a therapeutic agent in the treatment of cancer. Moreover, as many toxic insults relevant to this patent increase cancer risk (including, e.g., chemotherapeutic agents, radiation and agricultural chemicals), protection of cancer cells would also limit the use of 4-AP.

To investigate the effects of 4-AP treatment on cancer cells, 4-AP was co-applied with either paclitaxel or cisplatin in dose response curves extending over a >20-fold range to the widely studied E0771 murine breast cancer cell line and the A549 human lung cancer cell line. Cells were exposed to paclitaxel or cisplatin for 5 days in the presence or absence of 1 mM 4-AP. This concentration of 4-AP is typically used for in vitro studies, and greatly exceeds achievable concentrations in vivo. This higher dose would increase the likelihood of detecting protective activity.

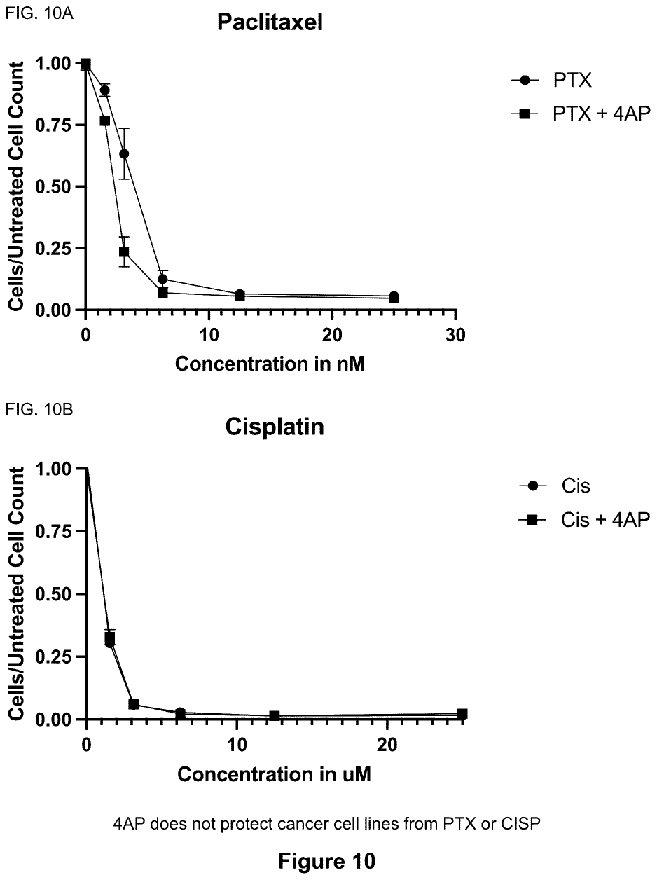

FIG. 10 shows that the presence of 4-AP does not protect cancer cells from cisplatin, and appears to even increase sensitivity to low doses of paclitaxel. Thus, this example reveals the surprising outcome that 4-AP's ability to protect multiple types of normal tissue from the toxic effects of cisplatin and paclitaxel is unusually selective and does not apply to cancer cells.

### Example 11. 4-AP Treatment Prevents Alterations in Kidney Function as Revealed by Analysis of Serum 4-AP Levels

In addition, Table 1 provides data showing that circulating 4-AP levels, as analyzed 1 hour after intraperitoneal injection, did not differ in mice treated with 4-AP alone versus 4-AP+PTX, thus indicating that 4-AP prevented PTX-induced changes in renal function. 4-AP was injected i.p. at the indicated time points after the beginning of treatment. Mice received PTX on day 0 of the experiment, and half of those mice also were injected with 4-AP at this time, and every day thereafter, at a dosage of 1/g/kg. Blood samples were drawn 3 and 7 days after the beginning of the experiment and were taken 1 hour after the injection of 4-AP. Circulating levels of 4-AP were determined by HPLC analysis. As shown, the amount of 4-AP detected in the circulation, whether at day 3 or day 7, did not differ between mice treated with 4-AP alone or PTX+4-AP. As 4-AP is eliminated by renal clearance, this further indicated that 4-AP prevented PTX-induced changes in kidney function.

TABLE 1
Circulating 4-AP levels in mice treated
with 4-AP alone versus 4-AP + PTX.

| | | Amount | | Amount |
|---|---|---|---|---|
| Group # | Date of Draw | (uM) | Date of Draw | (uM) |
| 4-AP only | 3 days post | 0.376 | 7 day 1st draw | 0.293 |
| 4-AP only | 3 days post | 0.486 | 7 day 1st draw | 0.140 |
| 4-AP only | 3 days post | 0.427 | 7 day 1st draw | 0.337 |
| 4-AP only | 3 days post | 0.298 | 7 day 1st draw | 0.376 |
| 4-AP only | 3 days post | 0.393 | 7 day 1st draw | 0.246 |
| PTX + 4-AP | 3 days post | 0.721 | 7 day 1st draw | 0.352 |
| PTX + 4-AP | 3 days post | 0.504 | 7 day 1st draw | 0.292 |
| PTX + 4-AP | 3 days post | 0.312 | 7 day 1st draw | 0.274 |
| PTX + 4-AP | 3 days post | 0.321 | 7 day 1st draw | 0.236 |
| PTX + 4-AP | 3 days post | 0.277 | 7 day 1st draw | 0.231 |
| PTX + H2O | 3 days post | −0.001 | 7 day 1st draw | 0.005 |
| PTX + H2O | 3 days post | −0.004 | 7 day 1st draw | −0.001 |
| PTX + H2O | 3 days post | −0.006 | 7 day 1st draw | −0.002 |
| PTX + H2O | 3 days post | 0.001 | 7 day 1st draw | −0.002 |
| PTX + H2O | 3 days post | −0.004 | 7 day 1st draw | 0.071 |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of preventing, alleviating, or treating a tissue damage or tissue dysfunction caused by a toxic insult in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

2. A method of reducing or reversing a tissue damage, oxidation damage, scarring, or any combination thereof in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and
the tissue damage, oxidation damage, scarring, or any combination thereof is caused by a toxic insult.

3. A method of restoring, improving, or enhancing at least a portion of tissue function in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and
the tissue function was reduced by a toxic insult.

4. The method of claim 3, wherein the tissue function is a motor function, sensory function, cognitive function, visual function, auditory function, kidney function, hematopoietic system function, normal skin function, salivary gland function, liver function, gall bladder function, gastrointestinal (GI) function, sexual function, or any combination thereof.

5. A method of restoring, improving, or enhancing myelination in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof, and
the myelination was reduced or modulated by a toxic insult.

6. A method of preventing, alleviating, or treating a mitochondrial damage or mitochondrial dysfunction caused by a toxic insult in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

7. A method of preventing, alleviating, or treating an axonal damage or axonal dysfunction caused by a toxic insult in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

8. A method of preventing, alleviating, or treating at least one gait abnormality caused by a toxic insult in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

9. A method of enhancing tissue regeneration, cell survival, or a combination thereof in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof, and
the subject was exposed to at least one toxic insult.

10. The method of claim 9, wherein the tissue is a kidney tissue, liver tissue, heart tissue, lung tissue, brain tissue, central nervous system tissue, peripheral nerve tissue, gastrointestinal tract tissue, gut tissue, visual system tissue, auditory system tissue, skin tissue, bladder tissue, reproductive system tissue, hematopoietic system tissue, musculoskeletal tissue, or any combination thereof.

11. A method of preventing, alleviating, or treating a chemotherapy-induced peripheral neuropathy (CIPN) in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

12. The method of claim 11, wherein the chemotherapy-induced peripheral neuropathy (CIPN) is a CIPN caused by taxane agent (P-CIPN), CIPN caused by cisplatin treatment (CisTPN), CIPN caused by an anti-cancer agent, CIPN caused by a platinum-based antineoplastic agent, CIPN caused by a vinca alkaloid agent, CIPN caused by an epothilone agent, CIPN caused by a proteasome inhibitor, CIPN caused by an immunomodulatory drug, or any combination thereof.

13. The method of any one of claim 1-12, wherein the at least one potassium channel blocker comprises 4-aminopyridine, a derivative of 4-aminopyridine, or a combination thereof.

14. The method of claim 13, wherein the derivative of 4-aminopyridine is a compound having the structure of Formula (I) 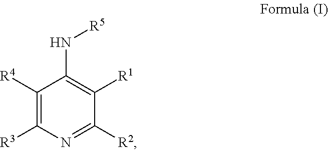
or an analog, a racemate, a tautomer, an isomer, an enantiomer, a diastereomer, a prodrug, or a pharmaceutically acceptable salt thereof;
wherein R¹, R², R³, R⁴, and R⁵ are each independently selected from hydrogen, halogen, C₁-C₆ alkyl, amine, hydroxyl, alkoxy, carboxyl, or any combination thereof, and
wherein R¹, R², R³, R⁴, and R⁵ are optionally substituted.

15. The method of any one of claims 1, 2, or 14, wherein the tissue damage is a kidney tissue damage, liver tissue damage, heart tissue damage, lung tissue damage, brain tissue damage, central nervous system damage, peripheral nerve tissue damage, peripheral neuropathy, nephropathy, chemotherapy-induced peripheral neuropathy (CIPN), radiation-induced peripheral neuropathy (RIPN), chemotherapy-induced nephrotoxicity (CINT), chemotherapy-induced neutropenia, radiation-induced neutropenia, gastrointestinal tract tissue damage, gut tissue damage, visual system tissue damage, auditory system tissue damage, skin tissue damage, bladder tissue damage, reproductive system tissue damage, hematopoietic system tissue damage, or any combination thereof.

16. The method of claim 15, wherein the chemotherapy-induced peripheral neuropathy (CIPN) is a CIPN caused by taxane agent (P-CIPN), CIPN caused by cisplatin treatment (CisIPN), CIPN caused by an anti-cancer agent, CIPN caused by a platinum-based antineoplastic agent, CIPN caused by a vinca alkaloid agent, CIPN caused by an epothilone agent, CIPN caused by a proteasome inhibitor, CIPN caused by an immunomodulatory drug, or any combination thereof.

17. The method of any one of claims 1, 2 or 14 wherein the tissue damage is a multi-tissue damage, multi-organ tissue damage, or any combination thereof.

18. The method of any one of claims 1-14, wherein the toxic insult is an acute toxic insult, chronic toxic insult, or any combination thereof.

19. The method of any one of claims 1-14, wherein the toxic insult comprises an exposure to damaging levels of radiation along the electromagnetic spectrum.

20. The method of any one of claims 1-14, wherein the toxic insult is a non-biological substance, non-naturally occurring compound, toxin, agent used in treating cancer, chemotherapy agent, biological response modifier, radiation, or any combination thereof.

21. The method of claim 20, wherein the chemotherapy agent comprises a platinum-based antineoplastic agent, vinca alkaloid agent, epothilone agent, taxane agent, proteasome inhibitor, immunomodulatory drug, or any combination thereof.

22. The method of claim 20, wherein the toxic insult comprises at least one environmental toxicant.

23. The method of claim 20, wherein the radiation comprises a radiation from a radioactive cancer treatment, radiation from a nuclear energy accident, radiation from a nuclear waste exposure, radiation from a use of radioactive substances in military applications, or any combination thereof.

24. The method of any one of claims 1-14, wherein the toxic insult comprises at least a first toxic insult and a second toxic insult,
wherein the first toxic insult is a chemotherapy agent, radiation, or a combination thereof; and
the second toxic insult is a chemotherapy agent, radiation, or a combination thereof.

25. The method of any one of claims 1-14, wherein the therapeutically effective amount of the pharmaceutical composition is administered to the subject before the toxic insult.

26. The method of any one of claims 1-14, wherein the therapeutically effective amount of the pharmaceutical composition is administered to the subject at the time of the toxic insult.

27. The method of any one of claims 1-14, wherein the therapeutically effective amount of the pharmaceutical composition is administered to the subject after the toxic insult.

28. The method of any one of claims 1-14, wherein the therapeutically effective amount of the pharmaceutical composition is administered to the subject more than once.

29. The method of any one of claims 1-14, wherein the therapeutically effective amount of the pharmaceutical composition is repeatedly administered to the subject for between about 1 day to about 100 years.

30. The method of any one of claims 1-14, wherein the therapeutically effective amount of the pharmaceutical composition is administered to the subject systematically, locally, or a combination thereof.

31. The method of any one of claims 1-14, wherein the therapeutically effective amount of the pharmaceutical composition is administered to the subject by an intraperitoneal injection, intravenous injection, intramuscular injection, intrathecal injection, subcutaneous injection, sublingual administration, inhalation, oral administration, transdermal administration, administration to an outer portion of the body in the form of a liquid, administration to an outer portion of the body in the form of a salve, administration to an outer portion of the body in the form of a bandage, or any combination thereof.

32. The method of any one of claims 1-14, wherein the therapeutically effective amount of the pharmaceutical composition is administered to the subject at a dose of between about 1 mg/day to about 1,000 mg/day of the potassium channel blocker.

33. The method of claim 32, wherein the therapeutically effective amount of the pharmaceutical composition is administered to the subject at a dose of between about 2.5 mg/day to about 40 mg/day of the potassium channel blocker.

34. The method of claim 32, wherein the therapeutically effective amount of the pharmaceutical composition is administered to the subject at a dose of between about 40 mg/day to about 100 mg/day of the potassium channel blocker.

35. The method of any one of claims 1-14, wherein the therapeutically effective amount of the pharmaceutical composition is co-administered with at least one anticonvulsant agent or a composition thereof.

36. The method of claim 35, wherein the at least one anticonvulsant agent is barbiturate, benzodiazepine, bromide, carbamate, carboxamide, fatty acid, fructose or a derivative thereof, y-aminobutyric acid (GABA) or an analog thereof, hydantoin, oxazolidinedione, proprionate, pyrimidinedione, pyrrolidine, succinimide, sulfonamide, triazine, urea, valproylamide, or any combination thereof.

37. The method of any one of claims 1-36, wherein the method further enhances a repair or regeneration of endogenous stem cells, enhances a repair or regeneration of transplanted stem cells, enhances a repair or regeneration of progenitor cells, promotes a neural cell generation, enhances cell survival, reduces scarring, decreases lesion size, decreases oxidative damage, or any combinations thereof.

38. A method of identifying a subject responsive to a 4-aminopyridine administration to prevent, alleviate, or treat a tissue damage or tissue dysfunction caused by a toxic insult in a subject in need thereof, wherein the method comprises the steps of:
a) administering to the subject between 1 to 5 therapeutically effective amounts of a pharmaceutical composition comprising a 4-aminopyridine, derivative of 4-aminopyridine, or a combination thereof,
b) evaluating the symptoms of the tissue damage or tissue dysfunction caused by the toxic insult in the subject; and
c) identifying the subject as responsive to 4-aminopyridine administration to prevent, alleviate, or treat the tissue damage or tissue dysfunction caused by a toxic insult when the symptoms of the tissue damage or tissue dysfunction caused by the toxic insult in the subject improved.
